(19) United States
(12) Patent Application Publication
(10) Pub. No.: US 2026/0242388 A1
(43) Pub. Date: Aug. 20, 2026

(54) NBD1 MODULATORS AND METHODS OF USING THE SAME

(71) Applicants: Sionna Therapeutics Inc., Waltham, MA (US); Genzyme Corporation, Cambridge, MA (US)

(72) Inventors: Junkai Liao, Andover, MA (US); Mark Munson, Acton, MA (US); Sukanthini Thurairatnam, Dover, MA (US); Bradford Hirth, Littleton, MA (US); Zhongli Gao, Waltham, MA (US); Gregory Donald Hurlbut, Boxborough, MA (US); Jinyu Liu, Union City, CA (US); Michael Kothe, Medway, MA (US); George Topalov, Pittsburgh, PA (US); John E. Macor, Washington Crossing, PA (US); Yi Li, Weston, MA (US); Andrew Good, Bideford (GB)

(21) Appl. No.: 19/160,779

(22) PCT Filed: Mar. 1, 2024
(86) PCT No.: PCT/US2024/018153

§ 371 Date: Aug. 28, 2025

Related U.S. Application Data
(60) Provisional application No. 63/449,486, filed on Mar. 2, 2023.

(51) Int. Cl. C07D 491/052; A61K 31/4162; A61K 31/437; A61K 31/4985; A61K 31/55; C07D 403/12; C07D 487/04

(52) U.S. Cl. CPC C07D 491/052; A61K 31/4162; A61K 31/437; A61K 31/4985; A61K 31/55; C07D 403/12; C07D 487/04

(57) ABSTRACT

The present disclosure includes, among other things, CFTR modulators, pharmaceutical compositions, and methods of making and using the same.

This application claims the benefit of and priority to U.S. Provisional Patent Application No. 63/449,486, filed on Mar. 2, 2023, the disclosure of each of which is hereby incorporated by reference in its entirety for all purposes.

### BACKGROUND

Cystic fibrosis (CF), an autosomal recessive disorder, is caused by functional deficiency of the cAMP-activated plasma membrane chloride channel, cystic fibrosis transmembrane conductance regulator (CFTR), which results in pulmonary and other complications. The gene encoding CFTR has been identified and sequenced (See Gregory, R. J. et al. (1990) Nature 347:382-386; Rich, D. P. et al. (1990) Nature 347:358-362), (Riordan, J. R. et al. (1989) Science 245:1066-1073). CFTR, a member of the ATP binding cassette (ABC) superfamily is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate, and thiocyanate into and out of the cell. CFTR may have a regulatory role over other electrolyte channels, including the epithelial sodium channel ENaC.

In cystic fibrosis patients, the absence or dysfunction of CFTR leads to exocrine gland dysfunction and a multisystem disease, characterized by pancreatic insufficiency and malabsorption, as well as abnormal mucociliary clearance in the lung, mucostasis, chronic lung infection and inflammation, decreased lung function and ultimately respiratory failure.

While more than 1,900 mutations have been identified in the CFTR gene, a detailed understanding of how each CFTR mutation may impact channel function is known for only a few. (Derichs, European Respiratory Review, 22:127, 58-65 (2013)). The most frequent CFTR mutation is the in-frame deletion of phenylalanine at residue 508 (ΔF508) in the first nucleotide binding domain (NBD1). Over 70% of cystic fibrosis patients have a deletion at residue 508 in at least one CFTR allele. The loss of this key phenylalanine renders NBD1 conformationally unstable at physiological temperature and compromises the integrity of the interdomain interface between NDB1 and CFTR's second transmembrane domain (ICL4). The ΔF508 mutation causes production of misfolded CFTR protein which, rather than traffic to the plasma membrane, is instead retained in the endoplasmic reticulum and targeted for degradation by the ubiquitin-proteasome system.

The loss of a functional CFTR channel at the plasma membrane disrupts ionic homeostasis and airway surface hydration leading to reduced lung function. Reduced periciliary liquid volume and increased mucus viscosity impede mucociliary clearance resulting in chronic infection and inflammation. In the lung, the loss of CFTR-function leads to numerous physiological effects downstream of altered anion conductance that result in the dysfunction of additional organs such as the pancreas, intestine and gall bladder.

By studying the mechanistic aspects of CFTR misfolding and corrections, small molecules have been identified as CF modulators, that can act as stabilizers.

Despite the identification of compounds that modulate CFTR, there is no cure for this fatal disease and identification of new compounds and new methods of therapy are needed as well as new methods for treating or lessening the severity of cystic fibrosis and other CFTR mediated conditions and diseases in a patient.

### SUMMARY

The present disclosure includes a compound of formula I:

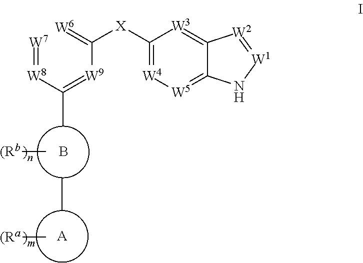

or a pharmaceutically acceptable salt thereof. Additionally, the present disclosure includes, among other things, pharmaceutical compositions, methods of using and methods of making a compound of formula I.

### DETAILED DESCRIPTION

In some embodiments, the present disclosure includes a compound of Formula I:

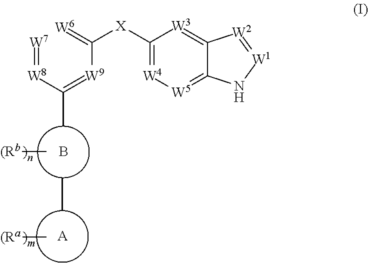

or a pharmaceutically acceptable salt thereof

wherein
W¹ is selected from the group consisting of —C(H)═, and —N═;
W² is selected from the group consisting of —C(H)═, —C(R^{d})═, and —N═;
W³ is selected from the group consisting of —C(H)═, —C(R^{d})═, and —N═;
W⁴ is selected from the group consisting of —C(H)═, —C(R^{d4})═, and —N═;
W⁵ is selected from the group consisting of —C(H)═, —C(R^{d5})═, and —N═;
W⁶ is selected from the group consisting of —C(H)═, —C(R^{∘})═, and —N═;
W⁷ is selected from the group consisting of —C(H)═, —C(R^{∘})═, and —N═;
W⁸ is selected from the group consisting of —C(H)═, —C(R^{∘})═, and —N═;
W⁹ is selected from the group consisting of —C(H)═, —C(R^{c9})═, and —N═;
Ring A is optionally substituted phenyl or 5-10 membered heteroaryl;
Ring B is an optionally substituted 8-10 membered fused heteroaryl;
each R^{a} is independently selected from the group consisting of halogen, oxo —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl,
   wherein each R^{a} is independently substituted with 0-4 instances of R^{aa},
each R^{aa} is independently selected from the group consisting of halogen, oxo, —COOH, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, —SO(NR²)R¹, —SO₂N(R²)R¹, and —N(H)C(O)N(R¹)₂,
   wherein two instances of R^{aa} are optionally taken together with any intervening atoms to form an optionally substituted 5-6 membered heterocyclyl ring;
each R^{b} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl,
   wherein two instances of R^{b} are optionally taken together with any intervening atoms to form an optionally substituted 5-6 membered carbocyclyl or optionally substituted 5-6 membered heterocyclyl ring;
each R^{c} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{d} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl; R^{c9} is halogen;
R^{d4} is halogen;
R^{d5} is halogen;
each R¹ is independently selected from the group consisting of hydrogen, —(CH₂)₁₋₃R², —C(O)R², —(CH₂)₁₋₃OR², optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
   wherein two instances of R¹ are optionally taken together with any intervening atoms to form an optionally substituted 3-7 membered heterocyclyl ring;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
X is selected from the group consisting of —O—, —S—, —S(O)—, —S(O)₂—; —SO(NR²)—, —C(R¹)(R²)—, and —C(O)—;
n is 0, 1, 2, or 3; and
m is 0, 1, 2, or 3.

The present disclosure includes a compound of Formula (I-a), (I-b), (I-c), (I-d), (I-e), or (I-f):

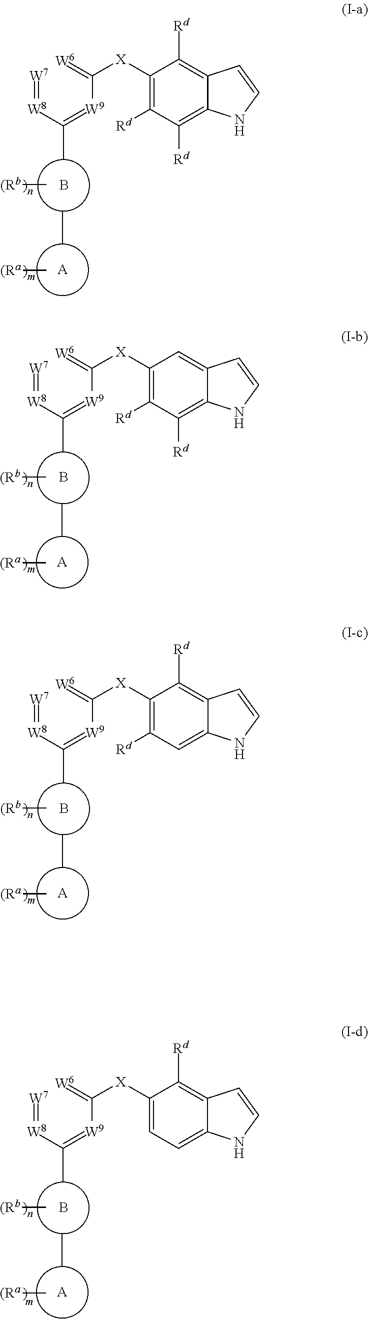

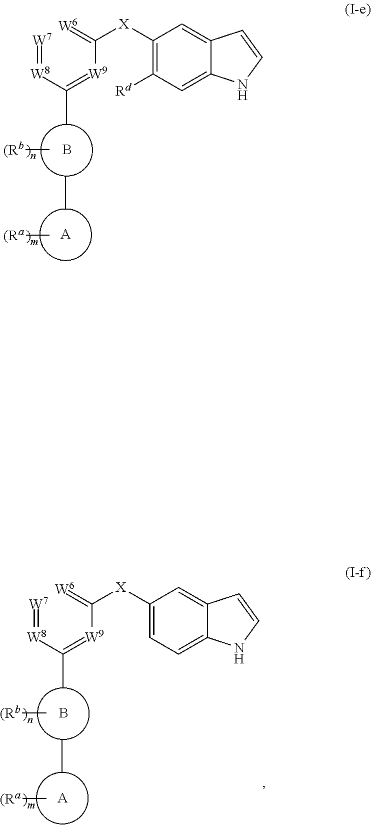

or a pharmaceutically acceptable salt thereof.

### X

In some embodiments, X is selected from the group consisting of —O—, —S—, —S(O)—, —S(O)₂—, —C(R¹)(R²)—, —C(O)—, and —CH(OH)—. In some embodiments, X is —O—. In some embodiments, X is —S—. In some embodiments, X is —S(O)—. In some embodiments, X is —S(O)₂—. In some embodiments, X is —CH₂—. In some embodiments, X is —C(H)(CH₃)—. In some embodiments, X is —C(O)—. In some embodiments, X is —CH(OH)—.

### Ring A

In some embodiments, Ring A is optionally substituted 5-membered heteroaryl, containing 1-2 heteroatoms selected from N, S, and O. In some embodiments, Ring A is an optionally substituted 5-membered heteroaryl selected from the group consisting of thiophenyl, pyrazolyl, pyrrolyl, and thiazolyl. In some embodiments, Ring A is optionally substituted thiophenyl. In some embodiments, Ring A is pyrazolyl. In some embodiments, Ring A is optionally substituted thiazolyl.

In some embodiments, Ring A is selected from the group consisting of

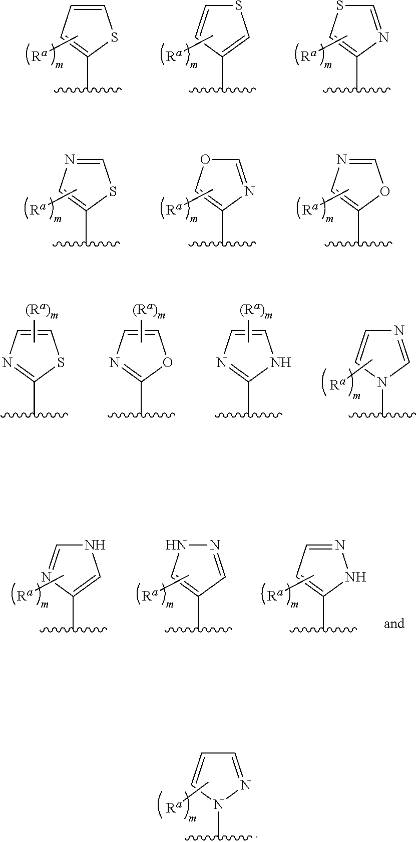

In some embodiments, Ring A is selected from the group consisting of

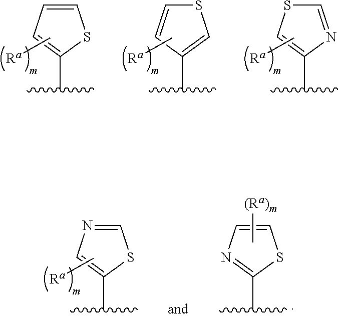

### Ring B

In some embodiments, Ring B is an optionally substituted 8-10 membered fused heteroaryl. In some embodiments, Ring B is selected from the group consisting of

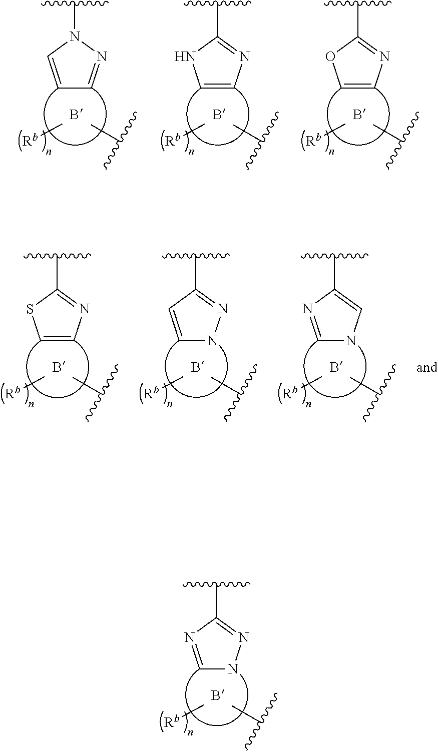

wherein B′ is selected from the group consisting of optionally substituted 5-7 carbocyclyl, optionally substituted 5-7 heterocyclyl, and optionally substituted 5-7 heteroaryl.

In some embodiments, Ring B is selected from the group consisting of

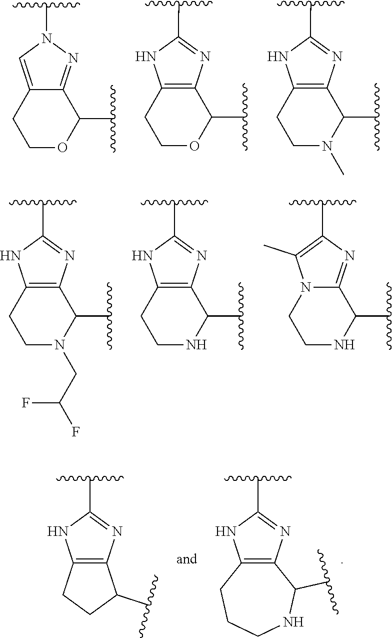

### R^{a}

In some embodiments, each each R^{a} is selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{a} is independently substituted with 0-4 instances of R^{aa}. In some embodiments, each R^{a} is independently selected from halogen, optionally substituted C₁-C₆ alkyl, and optionally substituted C₁-C₆ alkenyl, wherein each R^{a} is independently substituted with 0-4 instances of R^{aa}. In some embodiments, wherein each R^{a} is independently —CH₂COOH, —CH₂CH₂COOH, and —C(H)═C(H)—COOH. In some embodiments, wherein R^{a} is —CH₂COOH. In some embodiments, wherein R^{a} is —CH₂CH₂COOH. In some embodiments, wherein R^{a} is —C(H)═C(H)—COOH. In some embodiments, R^{a} is independently selected from the group consisting of fluoro, —CH₂CH₂COOH, —CH₂CH(Me)CO₂H, and —CH₂CH(OH)CH₂(OH). In some embodiments, R^{a} is halogen. In some embodiments, R^{a} fluoro. In some embodiments, R^{a} is —CH₂CH(Me)CO₂H. In some embodiments, R^{a} is —CH₂CH(OH)CH₂(OH). In some embodiments, R^{a} is —CH₂CH₂CO₂Et.

### R^{b}

In some embodiments, each R^{b} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO₂N(R²), —SO(NR²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl. In some embodiments, R^{b} is optionally substituted C₁-C₆ aliphatic. In some embodiments, R^{b} is optionally substituted C₁-C₆ alkyl. In some embodiments, R^{b} is optionally substituted C₁-C₃ alkyl. In some embodiments, R^{b} is optionally substituted methyl. In some embodiments, two instances of R^{b} are taken together, with any intervening atoms to form a 5-7 members optionally substituted carboxylic or heteroaryl ring. In some embodiments, R^{b} is —N(R¹)₂. In some embodiments, R^{b} is —N(H)CH₂CH₂OH. In some embodiments, R^{b} is piperidonyl. In some embodiments, R^{b} is pyrrolidinonyl.

### R^{c}

In some embodiments, each R^{c} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO₂N(R²), —SO(NR²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{c} is independently substituted with 0-4 instances of R^{aa}.

In some embodiments, R^{c} is halogen. In some embodiments, R¹ is fluoro.

### R^{d}

In some embodiments, each R^{d} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO₂N(R²), —SO(NR²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{d} is independently substituted with 0-4 instances of R^{aa}. In some embodiments, R^{d} is independently selected from the group consisting of halogen, —OR¹, —SR¹, —C(O)N(R¹)₂, —N(H)C(O)R¹, —SO₂R¹, —SO₂N(R²), —SO(NR²)R¹, and optionally substituted C₁-C₆ aliphatic, wherein each R^{d} is independently substituted with 0-4 instances of R^{aa}. In some embodiments, each R^{d} is halogen. In some embodiments, R^{d} is —N(H)C(O)R¹. In some embodiments, R^{d} is —CH₂OH. In some embodiments, R^{d} is —COOH. In some embodiment R^{d}—S(O)₂CH₃.

In some embodiments, R^{d4} is halogen. In some embodiments, R^{d4} is fluoro.

In some embodiments, R^{d5} is halogen. In some embodiments, R^{d5} is fluoro.

In some embodiments, the present disclosure includes compounds listed in Table 1.

TABLE 1

| Compound No. | Structure |
|---|---|
| 1 | 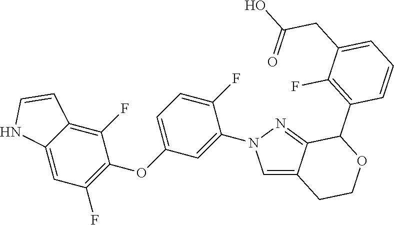 [SMILES: O=C(O)Cc1cccc(C2OCCc3cn(-c4cc(Oc5c(F)cc6[nH]ccc6c5F)ccc4F)nc32)c1F] |
| 2 | 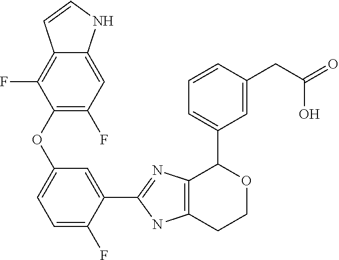 [SMILES: O=C(O)Cc1cccc(C2OCCc3[nH]c(-c4cc(Oc5c(F)cc6[nH]ccc6c5F)ccc4F)nc32)c1] |
| 3 | 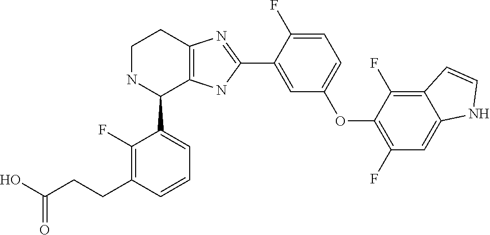 [SMILES: O=C(O)CCc1cccc([C@H]2NCCc3nc(-c4cc(Oc5c(F)cc6[nH]ccc6c5F)ccc4F)[nH]c32)c1F] |
| 4 | 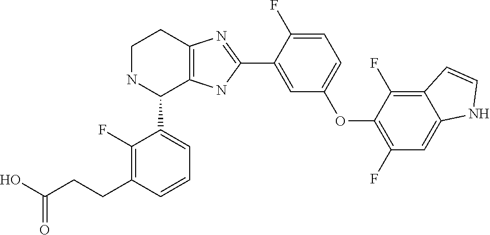 [SMILES: O=C(O)CCc1cccc([C@@H]2NCCc3nc(-c4cc(Oc5c(F)cc6[nH]ccc6c5F)ccc4F)[nH]c32)c1F] |
| 5 | 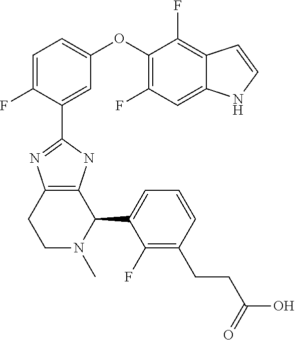 [SMILES: CN1CCc2nc(-c3cc(Oc4c(F)cc5[nH]ccc5c4F)ccc3F)[nH]c2[C@H]1c1cccc(CCC(=O)O)c1F] |
| 6 | 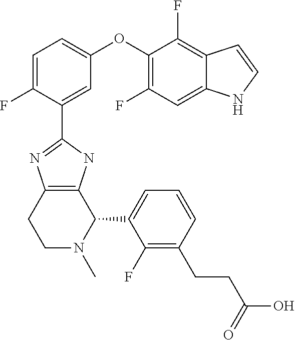 [SMILES: CN1CCc2nc(-c3cc(Oc4c(F)cc5[nH]ccc5c4F)ccc3F)[nH]c2[C@@H]1c1cccc(CCC(=O)O)c1F] |
| 7 | 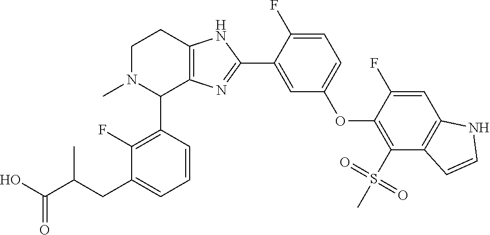 [SMILES: CC(Cc1cccc(C2c3nc(-c4cc(Oc5c(F)cc6[nH]ccc6c5S(C)(=O)=O)ccc4F)[nH]c3CCN2C)c1F)C(=O)O] |
| 8 | 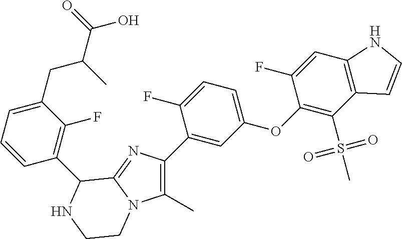 [SMILES: Cc1c(-c2cc(Oc3c(F)cc4[nH]ccc4c3S(C)(=O)=O)ccc2F)nc2n1CCNC2c1cccc(CC(C)C(=O)O)c1F] |
| 9 | 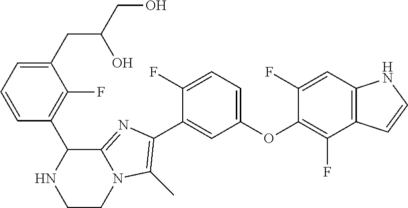 [SMILES: Cc1c(-c2cc(Oc3c(F)cc4[nH]ccc4c3F)ccc2F)nc2n1CCNC2c1cccc(CC(O)CO)c1F] |
| 10 | 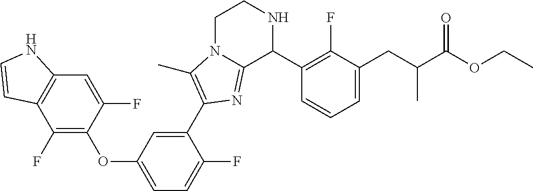 [SMILES: CCOC(=O)C(C)Cc1cccc(C2NCCn3c2nc(-c2cc(Oc4c(F)cc5[nH]ccc5c4F)ccc2F)c3C)c1F] |
| 11 | 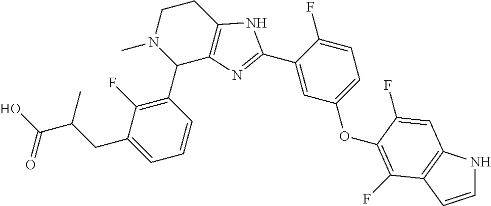 [SMILES: CC(Cc1cccc(C2c3nc(-c4cc(Oc5c(F)cc6[nH]ccc6c5F)ccc4F)[nH]c3CCN2C)c1F)C(=O)O] |
| 12 | 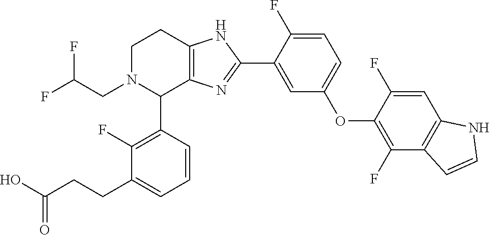 [SMILES: O=C(O)CCc1cccc(C2c3nc(-c4cc(Oc5c(F)cc6[nH]ccc6c5F)ccc4F)[nH]c3CCN2CC(F)F)c1F] |
| 13 | 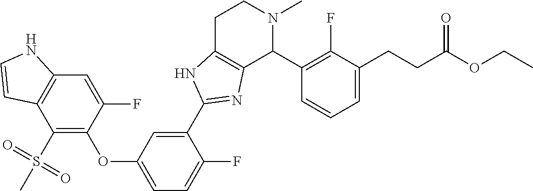 [SMILES: CCOC(=O)CCc1cccc(C2c3nc(-c4cc(Oc5c(F)cc6[nH]ccc6c5S(C)(=O)=O)ccc4F)[nH]c3CCN2C)c1F] |
| 14 | 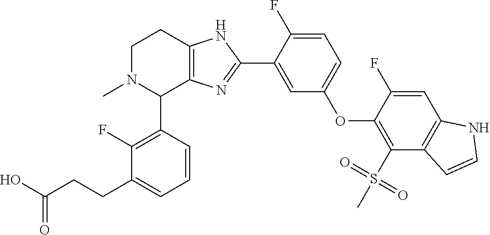 [SMILES: CN1CCc2[nH]c(-c3cc(Oc4c(F)cc5[nH]ccc5c4S(C)(=O)=O)ccc3F)nc2C1c1cccc(CCC(=O)O)c1F] |
| 15 | 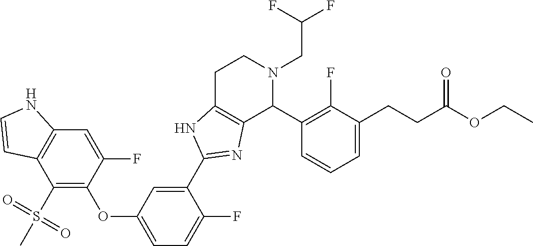 [SMILES: CCOC(=O)CCc1cccc(C2c3nc(-c4cc(Oc5c(F)cc6[nH]ccc6c5S(C)(=O)=O)ccc4F)[nH]c3CCN2CC(F)F)c1F] |
| 16 | 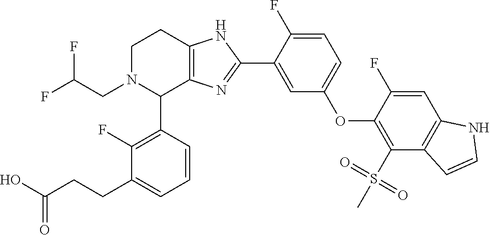 [SMILES: CS(=O)(=O)c1c(Oc2ccc(F)c(-c3nc4c([nH]3)CCN(CC(F)F)C4c3cccc(CCC(=O)O)c3F)c2)c(F)cc2[nH]ccc12] |
| 17 | 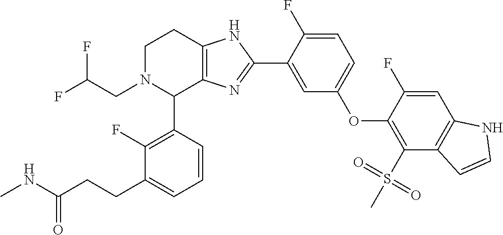 [SMILES: CNC(=O)CCc1cccc(C2c3nc(-c4cc(Oc5c(F)cc6[nH]ccc6c5S(C)(=O)=O)ccc4F)[nH]c3CCN2CC(F)F)c1F] |
| 18 | 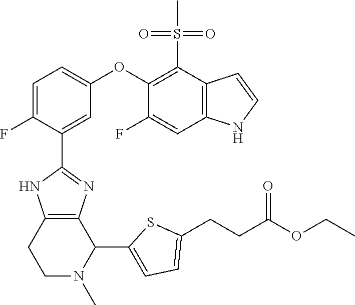 [SMILES: CCOC(=O)CCc1ccc(C2c3nc(-c4cc(Oc5c(F)cc6[nH]ccc6c5S(C)(=O)=O)ccc4F)[nH]c3CCN2C)s1] |
| 19 | 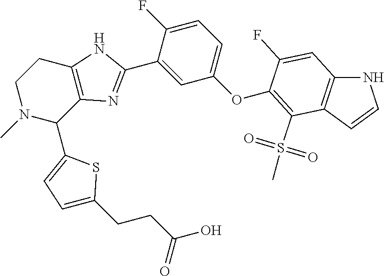 [SMILES: CN1CCc2[nH]c(-c3cc(Oc4c(F)cc5[nH]ccc5c4S(C)(=O)=O)ccc3F)nc2C1c1ccc(CCC(=O)O)s1] |
| 20 | 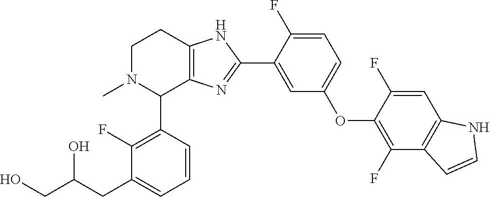 [SMILES: CN1CCc2[nH]c(-c3cc(Oc4c(F)cc5[nH]ccc5c4F)ccc3F)nc2C1c1cccc(CC(O)CO)c1F] |
| 21 | 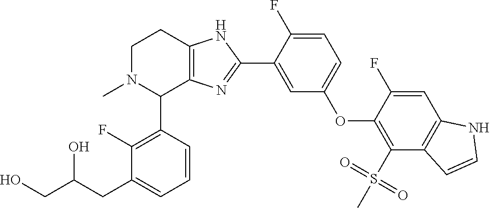 [SMILES: CN1CCc2[nH]c(-c3cc(Oc4c(F)cc5[nH]ccc5c4S(C)(=O)=O)ccc3F)nc2C1c1cccc(CC(O)CO)c1F] |
| 22 | 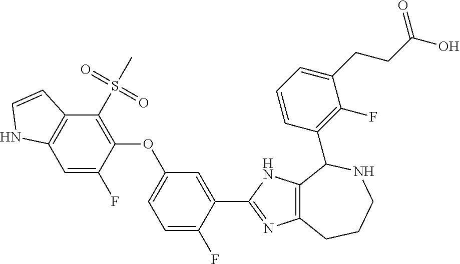 [SMILES: CS(=O)(=O)c1c(Oc2ccc(F)c(-c3nc4c([nH]3)C(c3cccc(CCC(=O)O)c3F)NCCC4)c2)c(F)cc2[nH]ccc12] |

or a pharmaceutically acceptable salt thereof.

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “haloalkyl” refers to a straight or branched alkyl group that is substituted with one or more halogen atoms.

The term “alkyl” as used herein is a branched or unbranched saturated hydrocarbon group having a specified number of carbon atoms. In some embodiments, alkyl refers to a branched or unbranched saturated hydrocarbon group having three carbon atoms (C₃). In some embodiments, alkyl refers to a branched or unbranched saturated hydrocarbon group having six carbon atoms (C₆). In some embodiments, the term “alkyl” includes, but is not limited to, methyl, ethyl, n-propyl, isopropyl, n-butyl, isobutyl, s-butyl, t-butyl, n-pentyl, isopentyl, s-pentyl, neopentyl, and hexyl.

As used herein, the term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group. The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, compounds of the disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —S(O)(NR^{∘})R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference.

Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat and/or diagnose the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of an CFTR-associated disease or disorder.

The terms “treat”, “treatment” or “treating” mean to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition. Without being bound by any theory, in some embodiments, treating includes augmenting deficient CFTR activity. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

As used herein, “CFTR” means cystic fibrosis transmembrane conductance regulator. Defects in the function of the CFTR ion channel result from loss of function mutations of CFTR. Such mutations lead to exocrine gland dysfunction, abnormal mucociliary clearance, and cause cystic fibrosis. The most common CFTR mutation in Cystic Fibrosis (CF) patients leads to the specific deletion of three nucleotides of the codon for phenylalanine at position 508. This mutation, which is found in ~70% of CF patients worldwide, is referred to as “ΔF508”. The ΔF508 mutation decreases the stability of the CFTR NBD1 domain and limits CFTR interdomain assembly. Since CF is an autosomal recessive disease, a CF patient harboring the ΔF508 CFTR mutation must also carry a second defective copy of CFTR. Approximately 2000 different CF-causing CFTR mutations have been identified in CF patients. CF patients harboring the ΔF508 CFTR mutation can be homozygous for that mutation (ΔF508/ΔF508). CF patients can also be ΔF508 heterozygous, if the second CFTR allele such patients carry instead contains a different CFTR loss of function mutation. Such CFTR mutations include, but are not limited to, G542X, G551D, N1303K, W1282X, R553X, R117H, R1162X, R347P, G85E, R560T, A455E, A1507, G178R, S549N, S549R, G551S, G970R, G1244E, S1251N, S1255P, and G1349D.

As used herein, the term “CFTR modulator” refers to a compound that increases the activity of CFTR. In certain aspects, a CFTR modulator is a CFTR corrector or a CFTR potentiator or a dual-acting compound having activities of a corrector and a potentiator.

As used herein, the term “CFTR corrector” refers to a compound that increases the amount of functional CFTR protein to the cell surface and thus enhances CFTR channel function. The CFTR correctors partially “rescue” misfolding of CFTR, thereby enabling the maturation and functional expression of CFTR protein harboring a CF causing mutation on the cell surface. Examples of correctors include, but are not limited to, VX-809, VX-661, VX-152, VX-440, VX-983, and GLPG2222. Such compounds may interact directly with CFTR protein, modifying its folding and conformational maturation during synthesis.

As used herein, the term “CFTR potentiator” refers to a compound that increases the ion channel activity of CFTR protein located at the cell surface, resulting in enhanced ion transport. CFTR potentiators repair the defective channel functions caused by mutations. Examples of potentiators include, but are not limited to, ivacaftor (VX770), deuterated ivacaftor (CPT 656), genistein and GLPG1837.

As used herein, the term “CFTR pharmacological chaperone” (PC) refers to compounds that stabilize the CFTR protein in its native state by binding directly to the protein.

As used herein, the term “CFTR proteostasis regulator” (PR) refers to compounds that enhance the protein folding efficiency within the cell. PRs can alter the activity of transcriptional, folding and/or membrane trafficking machinery, as well as impeding the degradation of partially folded, but functional, conformers at the endoplasmic reticulum (ER) or plasma membrane.

As used herein, “CFTR disease or condition” refers to a disease or condition associated with deficient CFTR activity, for example, cystic fibrosis, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), chronic sinusitis, dry eye disease, protein C deficiency, A-beta.-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a provided compound, an additional therapeutic agent, and a pharmaceutically acceptable carrier, adjuvant, or vehicle.

### ALTERNATIVE EMBODIMENTS

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of the present disclosure and a pharmaceutically acceptable carrier, adjuvant, or vehicle. In some embodiments, the amount of compound in compositions contemplated herein is such that is effective to measurably modulate CFTR, or a mutant thereof, in a biological sample or in a patient. In certain embodiments, the amount of compound in compositions of this disclosure is such that is effective to measurably modulate CFTR, or a mutant thereof, in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, the amount of compound in compositions contemplated herein is such that is effective to measurably modulate a protein, particularly at CFTR, or a mutant thereof, in a biological sample or in a patient. In certain embodiments, the amount of compound in compositions of this disclosure is such that is effective to measurably modulate CFTR, or a mutant thereof, in a biological sample or in a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of the present disclosure may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of the present disclosure may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of the present disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of the present disclosure may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of the present disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

As discussed above, CFTR is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate and thiocyanate into and out of the cell. The most frequent CFTR mutation is the in-frame deletion of phenylalanine at residue 508 (ΔF508) in the first nucleotide binding domain (NBD1). The mutation has several deleterious effects on the production of CFTR in the ER, its correct folding, its movement to the plasma membrane and its normal function as an ion channel for the cell.

One such negative effect is that the NBD1 domain is partially or mis-folded which is recognized within the cell as an aberrant protein and tagged for disposal by ER-associated degradation (ERAD) via the ubiquitin-proteasome system (UPS). Should a partially or mis-folded CFTR protein emerge from the ER, the protein must travel to the plasma membrane through complex glycosylation in the Golgi compartment and be functionally inserted. In wild-type CFTR, only 20-40% of CFTR reaches the plasma membrane, indicating that CFTR has energetic instability of individual NBDs, a slow domain assembly, and relatively fast ERAD kinetics which all contribute to inefficient folding and sensitize CFTR to structural perturbations by mutations.

In wild-type CFTR, the NBD1 domain folds co-translationally while other domains fold post-translationally. Mutated ΔF508 CFTR has impaired NBD1 folding but its backbone structure and thermodynamic stability are similar to wild-type CFTR. With delayed folding kinetics, mutated ΔF508 CFTR NBD1 has an increased folding activation energy. Lack of proper folding results in hydrophobic residues being exposed to the surface of NBD1 which causes aggregation with other CFTR proteins. Thus, the aggregation temperature of mutated CFTR drops from 41° C. to 33° C. This level of instability creates a greater percentage of mis-folded mutant CFTR at physiological temperature (37° C. in humans). Mutant CFTR suffers from both kinetic and thermodynamic folding defects. CFTR stabilizers can address these folding defects, but complete energetic correction of mutant NBD1 folding has been shown to not result in the CFTR biosynthetic processing, underscoring the need for interface stability as well.

The disclosed CFTR correctors can interact with the NBD domain to stabilize the correct folded position R, such that CFTR is not labeled for elimination from the cell. The preservation of correct folding enables CFTR to function as a chloride ion channel at wild-type levels. In some embodiments, disclosed CFTR correctors can enhance the performance of wild-type CFTR.

CFTR stabilizers can function in combination with other therapeutic agents such as CFTR correctors that promote A508 CFTR exit from the ER and accumulation in the plasma membrane. Increasing the amount of CFTR cell surface expression can result in improved chloride conductance following channel activation by both potentiators and a cAMP agonist. Thus, disclosed herein are combinations of CFTR stabilizers with CFTR correctors and potentiators, optionally with cAMP agonists or another therapeutic agent as described below.

Disclosed herein are methods of treating deficient CFTR activity in a cell, comprising contacting the cell with a compound of the present disclosure, or a pharmaceutically acceptable salt thereof. In certain embodiments, contacting the cell occurs in a subject in need thereof, thereby treating a disease or disorder mediated by deficient CFTR activity.

Also, disclosed herein are methods of treating a disease or a disorder mediated by deficient CFTR activity comprising administering a compound of the present disclosure or a pharmaceutically acceptable salt thereof. In some embodiments, the subject is a mammal, preferably a human. In some embodiments, the disease is associated with the regulation of fluid volumes across epithelial membranes, particularly an obstructive airway disease such as CF or COPD.

Such diseases and conditions include, but are not limited to, cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear palsy, Pick's disease, several polyglutamine neurological disorders, Huntington's, spinocerebellar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

Such diseases and conditions include, but are not limited to, cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome. In some embodiments, a disease is cystic fibrosis.

Provided herein are methods of treating cystic fibrosis, comprising administering to a subject in need thereof, a compound as disclosed herein or a pharmaceutically acceptable salt thereof. Also provided herein are methods of lessening the severity of cystic fibrosis, comprising administering to a subject in need thereof, a compound as disclosed herein or a pharmaceutically acceptable salt thereof. In some embodiments, the subject is a human. In some embodiments, the subject is at risk of developing cystic fibrosis, and administration is carried out prior to the onset of symptoms of cystic fibrosis in the subject.

Provided herein are compounds as disclosed herein for use in treating a disease or condition mediated by deficient CFTR activity. Also provided herein are uses of a compound as disclosed herein for the manufacture of a medicament for treating a disease or condition mediated by deficient CFTR activity.

Provided herein are kits for use in measuring the activity of CFTR or a fragment thereof in a biological sample in vitro or in vivo. The kit can contain: (i) a compound as disclosed herein, or a pharmaceutical composition comprising the disclosed compound, and (ii) instructions for: a) contacting the compound or composition with the biological sample; and b) measuring activity of said CFTR or a fragment thereof. In some embodiments, the biological sample is biopsied material obtained from a mammal or extracts thereof; blood, saliva, urine, feces, semen, tears, other body fluids, or extracts thereof. In some embodiments, the mammal is a human.

Provided herein are compounds as disclosed herein for use in treating kidney disease. In some embodiments, a kidney disease is autosomal dominant polycystic kidney disease (ADPKD) or autosomal recessive polycystic kidney disease (ARPKD). In some embodiments, a kidney disease is autosomal dominant polycystic kidney disease (ADPKD). In some embodiments, a kidney disease is autosomal recessive polycystic kidney disease (ARPKD).

### Combination Treatments

As used herein, the term “combination therapy” means administering to a subject (e.g., human) two or more CFTR modulators, or a CFTR modulator and an agent such as antibiotics, ENaC inhibitors, GSNO (S-nitrosothiol, s-nitroglutathione) reductase inhibitors, and a CRISPR Cas correction therapy or system (as described in US 2007/0022507 and the like). In some embodiments, combination therapy includes administration of a compound described herein with a compound that modulates CFTR protein or ABC protein activities (e.g., as described in WO2018167690A1 and the like)

In certain embodiments, the method of treating a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

In certain embodiments, the method of preventing a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

Additional therapeutic agents include, for example, ENaC inhibitors, mucolytic agents, modulators of mucus rheology, bronchodilators, antibiotics, anti-infective agents, anti-inflammatory agents, ion channel modulating agents, therapeutic agents used in gene or mRNA therapy, agents that reduce airway surface liquid and/or reduce airway surface PH, CFTR correctors, and CFTR potentiators, or other agents that modulate CFTR activity. Other therapeutics include liposomal composition components such as those described in WO2012/170889, hybrid oligonucleotides that facilitate RNA cleavage such as those described in WO2016/130943, and single stranded oligonucleotides that modulate gene expression as described in WO2016/130929.

In some embodiments, at least one additional therapeutic agent is selected from one or more CFTR modulators, one or more CFTR correctors and one or more CFTR potentiators.

Non-limiting examples of additional therapeutics include VX-770 (Ivacaftor), VX-809 (Lumacaftor, 3-(6-(I-(2,2-5 difluorobenzo[d][1, 3]dioxol-5-yl)cyclopropanecarboxamido)-3-methylpyridin-2-yl) benzoic acid, VX-661 (Tezacaftor, I-(2,2-difluoro-1, 3-benzodioxol-5-yl)-N-[I-[(2R)-2,3-dihydroxypropyl]-6-fluoro-2-(2-hydroxy-1, I-dimethylethyl)-1H-indol-5-yl]-cyclopropanecarboxamide), VX-983, VX-152, VX-440, VX-445, VX-659, VX-371, Orkambi, Ataluren (PTC 124) (3-[5-(2-fluorophenyl)-1, 2,4-oxadiazol-3-yl]benzoic acid), PTI-130 (Proteostasis), PTI-801, PTI-808, PTI-428, N91115.74 (cavosonstat), QBW251 (Novartis) compounds described in WO2011113894, compounds N30 Pharmaceuticals (e.g., WO 2014/186704), deuterated ivacaftor (e.g., CTP-656 or VX-561), GLPG 2222, GLPG2451, GLPG3067, GLPG2851, GLPG2737, GLPG 1837 (N-(3-carbamoyl-5,5,7,7-tetramethyl-5,7-dihydro-4H-thieno[2,3-c]pyran-2-yl)-1H-pyrazole-5-carboxamide), GLPG 2665 (Galapagos), FDL 169 (Flatley Discovery lab), FDL 176, FDL438, FDL304, FD2052160, FD1881042, FD2027304, FD2035659, FD2033129, FD1860293, CFFT-Pot01, CFFT-Pot-02, P-1037, glycerol, phenylbutyrate, and the like.

Non-limiting examples of additional therapeutics include compounds disclosed in US Patent Application Nos. PCT/US20/63586, PCT/US20/63589, and PCT/US20/63590, each of which is incorporated by reference in its entirety.

Non-limiting examples of anti-inflammatory agents are N6022 (3-(5-(4-(1H-imidazol-I-yl)10 phenyl)-I-(4-carbamoyl-2-methylphenyl)-'H-pyrrol-2-yl) propanoic acid), Ibuprofen, Lenabasum (anabasum), Acebilustat (CTX-4430), LAU-7b, POL6014, docosahexaenoic acid, alpha-1 anti-trypsin, sildenafil. Additional therapeutic agents also include, but are not limited to a mucolytic agent, a modifier of mucus rheology (such as hypertonic saline, mannitol, and oligosaccharide based therapy), a bronchodilator, an anti-infective (such as tazobactam, piperacillin, rifampin, meropenem, ceftazidime, aztreonam, tobramycin, fosfomycin, azithromycin, amitriptyline, vancomycin, gallium and colistin), an anti-infective agent, an anti-inflammatory agent, a CFTR modulator other than a compound of the present disclosure, and a nutritional agent. Additional therapeutic agents can include treatments for comorbid conditions of cystic fibrosis, such as exocrine pancreatic insufficiency which can be treated with Pancrelipase or Liprotamase.

Examples of CFTR potentiators include, but are not limited to, Ivacaftor (VX-770), CTP-656, NVS-QBW251, FD1860293, GLPG2451, GLPG1837, and N-(3-carbamoyl-5,5,7,7-tetramethyl-5,7-dihydro-4H-thieno[2,3-c]pyran-2-yl)-1H-pyrazole-5-carboxamide. Examples of potentiators are also disclosed in publications: WO2005120497, WO2008147952, WO2009076593, WO2010048573, WO2006002421, WO2008147952, WO2011072241, WO2011113894, WO2013038373, WO2013038378, WO2013038381, WO2013038386, WO2013038390, WO2014180562, WO2015018823, and U.S. patent application Ser. Nos. 14/271,080, 14/451,619 and 15/164,317.

Non-limiting examples of correctors include Lumacaftor (VX-809), 1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanecarboxamide (VX-661), VX-983, GLPG2222, GLPG2665, GLPG2737, VX-152, VX-440, FDL169, FDL304, FD2052160, and FD2035659. Examples of correctors are also disclosed in US20160095858A1, and U.S. application Ser. Nos. 14/925,649 and 14/926,727.

In certain embodiments, the additional therapeutic agent is a CFTR amplifier. CFTR amplifiers enhance the effect of known CFTR modulators, such as potentiators and correctors. Examples of CFTR amplifier include PTI130 and PTI-428. Examples of amplifiers are also disclosed in publications: WO2015138909 and WO2015138934.

In certain embodiments, the additional therapeutic agent is an agent that reduces the activity of the epithelial sodium channel blocker (ENaC) either directly by blocking the channel or indirectly by modulation of proteases that lead to an increase in ENaC activity (e.g., serine proteases, channel-activating proteases). Exemplary of such agents include camostat (a trypsin-like protease inhibitor), QAU145, 552-02, GS-9411, INO-4995, Aerolytic, amiloride, AZD5634, and VX-371. Additional agents that reduce the activity of the epithelial sodium channel blocker (ENaC) can be found, for example, in PCT Publication No. WO2009074575 and WO2013043720; and U.S. Pat. No. 8,999,976.

In one embodiment, the ENaC inhibitor is VX-371.

In one embodiment, the ENaC inhibitor is SPX-101 (S18).

In certain embodiments, the combination of a compound of the present disclosure, with a second therapeutic agent may have a synergistic effect in the treatment of cancer and other diseases or disorders mediated by adenosine. In other embodiments, the combination may have an additive effect.

### EXEMPLIFICATION

### Analytical Procedures

¹H NMR spectra were recorded with a Bruker AC 400 MHz apparatus. Chemical shifts (6) are quoted in parts per million (ppm) and coupling constants (J) in hertz (Hz).

LC-MS spectra were obtained, unless noted otherwise, with a UPLC Acquity device of Waters for the liquid chromatography analysis, coupling with a ZMD (Waters) mass spectrometer. This system was piloted by MassLynx v4.1 software. Detection was made in UV at 220 nm.

Operational conditions for liquid chromatography analysis are as follows:
Column: Assentis Express C₁₈ 50×2.1 mm, 2.7μ supelco
Eluent: A: H₂O+0.02% TFA;
B: CH₃CN+0.014% TFA;
Gradient: T₀ min: 2% B, T₁ min: 98% B, T_{1.3} min: 98% B, T_{1.33} min: 2% B, T_{1.5} min: following injection;
Flow: 1 mL/min;
Temperature: 55° C.
SQD: ESI+30V
UV detection wavelength: 220 nm
Injection volume: 0.2 μl.

Preparatory HPLC purification was carried out under the following conditions:
1. Instrument: Gilson 281 (PHG011)
2. Column Xtimate C18 21.2*250 mm, 10 μm
3. Mobile Phase: A: water (10 mM NH₄HCO₃ spiked with 0.025% NH₃·H₂O); B: acetonitrile
4. Gradient: 5% B for 3 min, then 5-37% B in 10 min, stop at 18 min
5. Flow Rate: 30.00 ml/min
6. Detection Wavelength (nm): 214/254
7. Retention Time (min): 8

### Abbreviations

AcOH: acetic acid
AIBN: azobisisobutyronitrile
BINAP: 2,2′-bis(diphenylphosphino)-1,1′-binaphthyl
Boc: tert-butyloxycarbonyl
n-BuOH: n-butanol
DABAL-Me3: bis(trimethylaluminum)-1,4-diazabicyclo[2.2.2]octane adduct
DBU: 1,8-dDiazabicyclo|5.4.0|undec-7-ene
DCM: dichloromethane
DCE: 1,2-dichloroethane
DEA: diethyl amine
DIPEA: N,N-diisopropylethylamine
DMAP: 4-dimethylaminopyridine
DMF: N,N-dimethylformamide
DMF-DMA: N,N-dimethylformamide dimethyl acetal
DMSO: dimethyl sulfoxide
dppf: 1,1′-bBis(diphenylphosphino)ferrocene
DTT: dithiothreitol
Ee: enantiomeric excess
Eq: equivalents
ESI: electron spray ionization
EtOAc: ethyl acetate
EtOH: ethanol
FA: formic acid
h: hour/hours
HATU: 1-[Bis(dimethylamino)methylene]-1H-1,2,3-triazolo[4,5-b]pyridinium 3-oxide hexafluorophosphate
HPLC: high performance liquid chromatography
In vacuo: under vacuum; under reduced pressure
LAH: lithium aluminum hydride
LC-MS: liquid chromatography-mass spectrometry
LDA: lithium diisopropylamide
LHMDS: lithium bis(trimethylsilyl)amide
MeOH: methanol
NBS: N-bromosuccinimide
NIS: N-iodosuccinimide
NMP: N-mMethyl-2-pyrrolidone
Pd/C: Palladium on carbon
Prep-HPLC: preparative HPLC
SFC: supercritical fluid chromatography
TEA: Triethylamine
TBAF: tetra-n-butylammonium fluoride
TBS: tert-butyldimethylsilyl
TFA: trifluoroacetic acid
TIPS: triisopropylsilyl
THF: tetrahydrofuran
THP: tetrahydropyran
TLC: thin layer chromatography
Ts: tosyl

### Example 1. Synthesis of 2-(3-(2-(5-((4,6-difluoro-1H-indol-5-yl)oxy)-2-fluorophenyl)-2,4,5,7-tetrahydropyrano[3,4-c]pyrazol-7-yl)-2-fluorophenyl)acetic acid

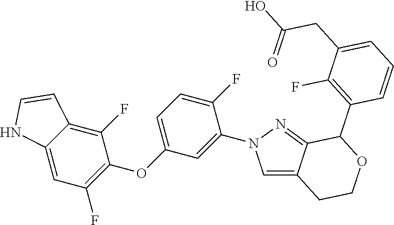 [SMILES: O=C(O)Cc1cccc(C2OCCc3cn(-c4cc(Oc5c(F)cc6[nH]ccc6c5F)ccc4F)nc32)c1F]

### 1,2,3-Trifluoro-4-methyl-5-nitrobenzene

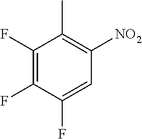

Step A: To a solution of 4-bromo-1,2,3-trifluoro-5-nitro-benzene (40 g, 0.157 mol) in dioxane/H₂O (4:1, 400 mL) was added methylboronic acid (47.2 g, 0.785 mol), K₂CO₃ (43.2 g, 0.314 mol) and Pd(dppf)Cl₂ (5.76 g, 7.85 mmol) under an N2 atmosphere. The mixture was stirred at 100° C. for 16 hours. The reaction was quenched with water (300 mL) and extracted with EtOAc (60 mL×3). The combined organic phase was washed with brine (200 mL), dried over Na₂SO₄ and concentrated. The residue was purified by silica gel column chromatography, eluting with petroleum ether, to afford the 1,2,3-trifluoro-4-methyl-5-nitro-benzene (16.7 g, 44%) as a colorless oil. ¹H NMR (400 MHz, CDCl₃) δ 7.76 (s, 1H), 2.53 (m, 3H) ppm.

### 5-(2,6-Difluoro-3-methyl-4-nitrophenoxy)-2-fluoroaniline

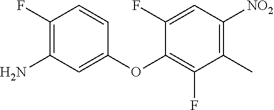 [SMILES: Cc1c([N+](=O)[O-])cc(F)c(Oc2ccc(F)c(N)c2)c1F]

Step B: To a solution of 1,2,3-trifluoro-4-methyl-5-nitro-benzene (16.7 g, 0.088 mol) in DMF (170 ml) was added K₂CO₃ (24 g, 0.18 mol) and 3-amino-4-fluoro-phenol (12.22 g, 0.096 mol). The mixture was stirred at room temperature overnight. The reaction was quenched with water (300 mL) and extracted with EtOAc (60 mL×3). The combined organic phase was washed with brine (200 mL), dried over Na₂SO₄ and concentrated. The residue was purified by silica gel column chromatography, eluting with 30:1 petroleum ether:ethyl acetate, to afford 5-(2,6-difluoro-3-methyl-4-nitro-phenoxy)-2-fluoro-aniline (22.7 g, 87%) as a yellow solid. MS (ESI): m/z 299.1 [M+H]⁺.

### 1,3-Difluoro-2-(4-fluoro-3-iodophenoxy)-4-methyl-5-nitrobenzene

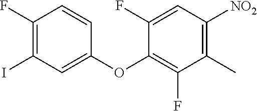 [SMILES: Cc1c([N+](=O)[O-])cc(F)c(Oc2ccc(F)c(I)c2)c1F]

Step C: To a solution of 5-(2,6-difluoro-3-methyl-4-nitro-phenoxy)-2-fluoro-aniline (22.7 g, 0.076 mol) in 3M aqueous HCl (230 mL) was added NaNO₂ (8 g, 0.114 mol) in H₂O (10 mL) at 0° C. The mixture was stirred at room temperature for 2 hours. The mixture was cooled to 0° C. and KI (51 g, 0.305 mol) in H₂O (20 mL) was added dropwise to the reaction. The mixture was stirred at room temperature for 16 hours. The reaction was quenched with water (300 mL) and extracted with EtOAc (60 mL×3). The combined organic phase was washed with brine (200 mL), dried over Na₂SO₄ and concentrated. The residue was purified by silica gel column chromatography, eluting with 50:1 petroleum ether:ethyl acetate to afford 1,3-difluoro-2-(4-fluoro-3-iodo-phenoxy)-4-methyl-5-nitro-benzene (22.1 g, 71%) as a red solid. MS (ESI): m/z 409.7 [M+H]+.

### 4,6-Difluoro-5-(4-fluoro-3-iodophenoxy)-1H-indole

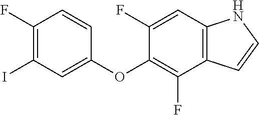 [SMILES: Fc1ccc(Oc2c(F)cc3[nH]ccc3c2F)cc1I]

Step D: To a solution of 1,3-difluoro-2-(4-fluoro-3-iodo-phenoxy)-4-methyl-5-nitro-benzene (20.0 g, 0.049 mol) in DMF (80 mL) was added DMF-DMA (58.2 g, 0.49 mol). The mixture was stirred at 100° C. for 1 hour. The mixture was quenched with water (50 mL), extracted with EtOAc (50 mL×3), dried over Na₂SO₄ and concentrated. The residue was dissolved in AcOH (120 mL) and toluene (120 mL), and Fe powder (28 g, 0.49 mol) was added. The mixture was stirred at 50° C. 16 hours. The mixture was diluted with water (100 mL), extracted with EtOAc (50 mL×3), dried over Na₂SO₄ and concentrated. The residue was purified by silica gel column chromatography, eluting with 10:1 petroleum ether:ethyl acetate, to afford 4,6-difluoro-5-(4-fluoro-3-iodo-phenoxy)-1H-indole (13 g, 68%) as a red solid. MS (ESI): m/z 390.0 [M+H]⁺.

### 4,6-Difluoro-5-(4-fluoro-3-iodophenoxy)-1-tosyl-1H-indole

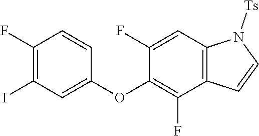 [SMILES: Cc1ccc(S(=O)(=O)n2ccc3c(F)c(Oc4ccc(F)c(I)c4)c(F)cc32)cc1]

Step E: To a solution of 4,6-difluoro-5-(4-fluoro-3-iodo-phenoxy)-1H-indole (13 g, 0.03342 mol) in DMF (130 mL) was added NaH (60% dispersion in oil, 2.4 g, 0.059 mol) at 0° C. The reaction mixture was stirred at 0° C. for 10 minutes. TsCl (11.2 g, 0.059 mol) was added in portions at 0° C. The reaction mixture was stirred for 20 minutes. Water (100 mL) was added, and the mixture extracted with EtOAc (50 mL×3), the extracts were combined and dried over Na₂SO₄ and concentrated. The residue was purified by silica gel column chromatography, eluting with 50:1 petroleum ether:ethyl acetate to afford 4,6-difluoro-5-(4-fluoro-3-iodo-phenoxy)-1-(p-tolylsulfonyl)indole (10 g, 56%) as a white solid. MS (ESI): m/z 566.2 [M+Na]⁺.

### 1-(Tetrahydro-2H-pyran-2-yl)-4-(2-((tetrahydro-2H-pyran-2-yl)oxy)ethyl)-1H-pyrazole

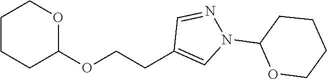 [SMILES: c1nn(C2CCCCO2)cc1CCOC1CCCCO1]

Step F: To a solution of 2-(1H-pyrazol-4-yl)ethanol (3.00 g, 26.8 mmol) in a 10:1 EtOAc:DMF mixture (198.0 mL) was added 3,4-dihydropyran (2.44 mL, 26.8 mmol) and p-toluenesulfonic acid (0.508 g, 2.68 mmol). The mixture was stirred at 60° C. overnight under a nitrogen atmosphere. The reaction was quenched with water (10 mL) and extracted with EtOAc (20 mL×3). The combined organic phase was washed with brine (20 mL), dried over Na₂SO₄ and concentrated. The residue was purified by silica gel column chromatography, eluting with 4:1 petroleum ether:EtOAc to afford 1-tetrahydropyran-2-yl-4-(2-tetrahydropyran-2-yloxyethyl)pyrazole (5.00 g, 66%) as a light oil. MS (ESI): m/z 281.0 [M+H]⁺.

### (3-Bromo-2-fluorophenyl)(1-(tetrahydro-2H-pyran-2-yl)-4-(2-((tetrahydro-2H-pyran-2-yl)oxy)ethyl)-1H-pyrazol-3-yl)methanol

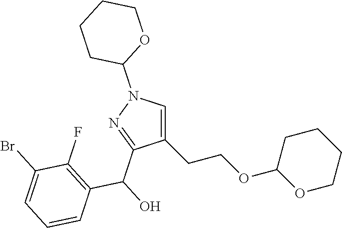 [SMILES: OC(c1cccc(Br)c1F)c1nn(C2CCCCO2)cc1CCOC1CCCCO1]

Step G: To a solution of 1-tetrahydropyran-2-yl-4-(2-tetrahydropyran-2-yloxyethyl)pyrazole (5.00 g, 17.8 mmol) in THF (100 mL) was added n-BuLi (2.50 M, 9.27 mL, 23.2 mmol) in THE at −75° C. The mixture was stirred at −75° C. for 2 hours under a nitrogen atmosphere. To the mixture was then added 3-bromo-2-fluoro-benzaldehyde (3.62 g, 0.0178 mol) at −75° C. and stirring at −75° C. was continued for 2 hours. The reaction was quenched with water (50 mL) and extracted with EtOAc (50 mL×3). The combined organic phase was washed with brine (50 mL), dried over Na₂SO₄ and concentrated. The residue was purified by silica gel column chromatography, eluting with 10:1 petroleum ether: ethyl acetate to afford (3-bromo-2-fluoro-phenyl)-[1-tetrahydropyran-2-yl-4-(2-tetrahydropyran-2-yloxyethyl)pyrazol-3-yl]methanol (7.00 g, 81%) as a light oil. MS (ESI): m/z 483, 485 [M+H]⁺.

### 7-(3-Bromo-2-fluoro-phenyl)-2,4,5,7-tetrahydropyrano[3,4-c]pyrazole

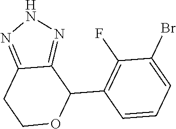 [SMILES: Fc1c(Br)cccc1C1OCCc2n[nH]nc21]

Step H: To a solution of (3-bromo-2-fluoro-phenyl)-[1-tetrahydropyran-2-yl-4-(2-tetrahydropyran-2-yloxyethyl)pyrazol-3-yl]methanol (5.00 g, 10.3 mmol) in xylenes (100 mL) was added para-toluenesulfonic acid (1.97 g, 10.3 mmol). The mixture was stirred at 120° C. for 24 hours under a nitrogen atmosphere. The reaction was quenched with water (50 mL) and extracted with EtOAc (50 mL×3). The combined organic phase was washed with brine (50 mL), dried over Na₂SO₄ and concentrated. The residue was purified by silica gel column chromatography, eluting with 10:1 petroleum ether:ethyl acetate, to afford 7-(3-bromo-2-fluoro-phenyl)-2,4,5,7-tetrahydropyrano[3,4-c]pyrazole (2.00 g, 65%) as a light oil. MS (ESI): m/z 297, 299 [M+H]⁺.

### 2-[2-Fluoro-3-(2,4,5,7-tetrahydropyrano[3,4-c]pyrazol-7-yl)phenyl]acetonitrile

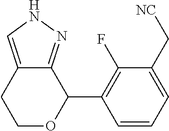 [SMILES: N#CCc1cccc(C2OCCc3c[nH]nc32)c1F]

Step I: To a solution of 7-(3-bromo-2-fluoro-phenyl)-2,4,5,7-tetrahydropyrano[3,4-c]pyrazole (1.50 g, 5.05 mmol) in 3:1 DMSO:water (15 mL) was added in a glove box, under an atmosphere of nitrogen, Pd(dppf)Cl₂ (0.246 g, 0.337 mmol), KF (0.587 g, 10.1 mmol) and 4-(4,4,5,5-tetramethyl-1,3,2-dioxaborolan-2-yl)isoxazole (1.48 g, 7.57 mmol). The mixture was stirred at 130° C. for 24 hours. The reaction was quenched with water (10 mL) and extracted with EtOAc (10 mL×3). The combined organic phase was washed with brine (50 mL), dried over Na₂SO₄ and concentrated. The residue was purified by silica gel column chromatography, eluting with 2:1 petroleum ether:ethyl acetate to afford 2-[2-fluoro-3-(2,4,5,7-tetrahydropyrano[3,4-c]pyrazol-7-yl)phenyl]acetonitrile (0.50 g, 38%) as a light oil. MS (ESI): m/z 258.1 [M+H]⁺.

### 2-[3-[2-[5-[4,6-Difluoro-]-(p-tolylsulfonyl)indol-5-yl]oxy-2-fluoro-phenyl]-5,7-dihydro-4H-pyrano[3,4-c]pyrazol-7-yl]-2-fluoro-phenyl]acetonitrile

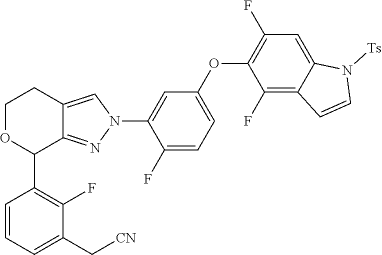 [SMILES: Cc1ccc(S(=O)(=O)n2ccc3c(F)c(Oc4ccc(F)c(-n5cc6c(n5)C(c5cccc(CC#N)c5F)OCC6)c4)c(F)cc32)cc1]

Step J: To a solution of 2-[2-fluoro-3-(2,4,5,7-tetrahydropyrano[3,4-c]pyrazol-7-yl)phenyl]acetonitrile (0.250 g, 0.972 mmol) in NMP (10.00 mL) was added in a glove box, under an atmosphere of nitrogen, 4,6-difluoro-5-(4-fluoro-3-iodo-phenoxy)-1-(p-tolylsulfonyl)indole (product from Step E, 0.686 g, 1.26 mmol), CuI (0.0369 g, 0.194 mmol), (1R,2R)-(−)-N,N′-dimethylcyclohexane-1,2-diamine (0.0552 g, 0.389 mmol) and Na₂CO₃ (0.206 g, 0.194 mmol). The mixture was stirred at 100° C. for 24 hours. The reaction was quenched with water (10 mL) and extracted with EtOAc (10 mL×3). The combined organic phase was washed with brine (10 mL), dried over Na₂SO₄ and concentrated. The residue was purified by silica gel column chromatography, eluting with 5:1 petroleum ether:ethyl acetate, to afford 2-[3-[2-[5-[4,6-difluoro-1-(p-tolylsulfonyl)indol-5-yl]oxy-2-fluoro-phenyl]-5,7-dihydro-4H-pyrano[3,4-c]pyrazol-7-yl]-2-fluoro-phenyl]acetonitrile (0.109 g, 16%) as a light oil. MS (ESI): m/z 672.3 [M]⁺.

### 2-(3-(2-(5-((4,6-Difluoro-1H-indol-5-yl)oxy)-2-fluorophenyl)-2,4,5,7-tetrahydropyrano[3,4-c]pyrazol-7-yl)-2-fluorophenyl)acetic acid

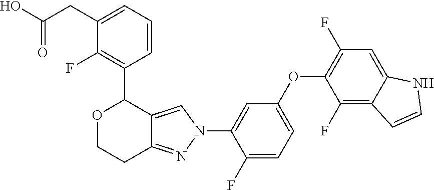 [SMILES: O=C(O)Cc1cccc(C2OCCc3nn(-c4cc(Oc5c(F)cc6[nH]ccc6c5F)ccc4F)cc32)c1F]

Step K: To a solution of 2-[3-[2-[5-[4,6-difluoro-1-(p-tolylsulfonyl)indol-5-yl]oxy-2-fluoro-phenyl]-5,7-dihydro-4H-pyrano[3,4-c]pyrazol-7-yl]-2-fluoro-phenyl]acetonitrile (0.100 g, 0.149 mmol) in an EtOH-water mixture (15.00 mL) was added aqueous KOH (3.00 M, 0.991 mL, 2.97 mmol). The mixture was stirred at 100° C. for 8 hours. The reaction pH was adjusted to ~7, then water (10 mL) added, and the resulting mixture extracted with EtOAc (10 mL×3). The combined organic phase was washed with brine (10 mL), dried over Na₂SO₄ and concentrated to afford 2-[3-[2-[5-[(4,6-difluoro-1H-indol-5-yl)oxy]-2-fluoro-phenyl]-5,7-dihydro-4H-pyrano[3,4-c]pyrazol-7-yl]-2-fluoro-phenyl]acetic acid (0.0380 g, 47%) as a white solid. MS (ESI): m/z 538.2 [M+H]⁺. ¹H NMR (400 MHz, MeOD-d₄): δ 7.94 (d, J=2.6 Hz, 1H), 7.31-7.20 (m, 4H), 7.14-7.10 (m, 2H), 7.04-7.00 (m, 1H), 6.92-6.84 (m, 1H), 6.54 (d, J=3.2 Hz, 1H), 6.08 (s, 1H), 4.16-4.10 (m, 1H), 3.90-3.82 (m, 1H), 3.72-3.60 (m, 2H), 2.98-2.89 (m, 1H), 2.84-2.75 (m, 1H) ppm.

### Example 2. Synthesis of 2-(3-(2-(5-((4,6-difluoro-1H-indol-5-yl)oxy)-2-fluorophenyl)-1,4,6,7-tetrahydropyrano[3,4-d]imidazol-4-yl)phenyl)acetic acid

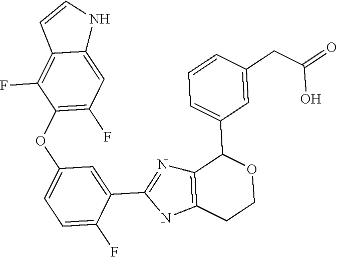 [SMILES: O=C(O)Cc1cccc(C2OCCc3[nH]c(-c4cc(Oc5c(F)cc6[nH]ccc6c5F)ccc4F)nc32)c1]

### 5-(2,6-Difluoro-4-nitrophenoxy)-2-fluorobenzonitrile

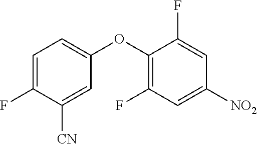 [SMILES: N#Cc1cc(Oc2c(F)cc([N+](=O)[O-])cc2F)ccc1F]

Step A: To a solution of 1,2,3-trifluoro-5-nitrobenzene (45 g, 0.254 mol) and 2-fluoro-5-hydroxybenzonitrile (38.3 g, 0.28 mol) in DMF (200 mL) was added K₂CO₃ (70 g, 0.5 mol). The resulting mixture was heated for 2 hours at 100° C. To the cooled reaction was added water (1500 mL), and the mixture extracted with EtOAc (1000 mL×2). The combined organic phase was washed with brine (500 mL×3), dried over anhydrous Na₂SO₄, filtered, and concentrated under reduced pressure to afford the desired product, 5-(2,6-difluoro-4-nitrophenoxy)-2-fluorobenzonitrile (74 g, 99%) as a yellow solid. ¹H NMR (500 MHz, DMSO-d₆): δ 8.03-7.98 (m, 2H), 7.28-7.23 (m, 2H), 7.21-7.19 (m, 1H) ppm.

### 5-(4-Amino-2,6-difluorophenoxyl)-2-fluorobenzonitrile

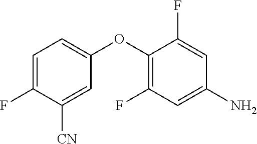 [SMILES: N#Cc1cc(Oc2c(F)cc(N)cc2F)ccc1F]

Step B: To a suspension of 5-(2,6-difluoro-4-nitrophenoxy)-2-fluorobenzonitrile (37 g, 126 mmol) in EtOH (450 mL) was added iron power (28.1 g, 543 mmol) and a solution of NH₄Cl (53.8 g, 1.01 mol) in water (150 mL). The resulting mixture was heated to reflux for 4 hours. The reaction mixture was filtered through Celite, and the filtrate was concentrated under reduced pressure to give a residue, which was dissolved in ethyl acetate (1 L). The organic phase was washed with water (200 mL×3), brine (200 mL), dried over anhydrous Na₂SO₄, filtered, and concentrated in vacuo to afford the desired product, 5-(4-amino-2,6-difluorophenoxy)-2-fluorobenzonitrile (33.2 g, quantitative yield) as a yellow solid. 1H NMR (500 MHz, CDCl₃) δ 7.23-7.20 (m, 1H), 7.14 (t, J=8.5 Hz, 1H), 7.08 (dd, J=5.0, 3.5 Hz, 1H), 6.33-6.28 (m, 2H), 3.90 (br s, 2H) ppm.

### 5-(4-Amino-2,6-difluoro-3-iodophenoxy)-2-fluorobenzonitrile

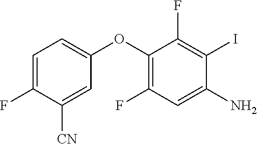 [SMILES: N#Cc1cc(Oc2c(F)cc(N)c(I)c2F)ccc1F]

Step C: A solution of 5-(4-amino-2,6-difluorophenoxy)-2-fluorobenzonitrile (33.2 g, 126 mmol) and N-iodosuccinimide (31.1 g, 126 mmol) in AcOH (250 mL) was stirred at room temperature for 1 hour. The solution was concentrated under reduced pressure. The residue was suspended in saturated aqueous NaHCO₃ (500 mL), and the resulting mixture was extracted with ethyl acetate (500 mL×3). The organic phase was washed with brine (200 mL×3), dried over anhydrous Na₂SO₄, filtered and concentrated. The residue was purified by silica gel column chromatography (eluting with 4/1 petroleum ether/ethyl acetate) to afford the desired product, 5-(4-amino-2,6-difluoro-3-iodophenoxy)-2-fluorobenzonitrile (45.5 g, 92%) as a yellow solid. ¹H NMR (500 MHz, DMSO-d₆): δ 7.63 (dd, J=5.0, 3.5 Hz, 1H), 7.49 (t, J=9.0 Hz, 1H), 7.39-7.36 (m, 1H), 6.63 (dd, J=13, 2.0 Hz, 1H), 5.88 (br s, 2H) ppm.

### 5-[4-Amino-2,6-difluoro-3-(2-trimethylsilylethynyl)phenoxy]-2-fluorobenzonitrile

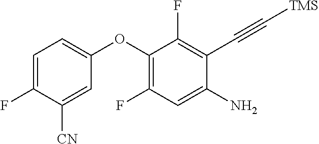 [SMILES: C[Si](C)(C)C#Cc1c(N)cc(F)c(Oc2ccc(F)c(C#N)c2)c1F]

Step D: To a solution of 5-(4-amino-2,6-difluoro-3-iodophenoxy)-2-fluorobenzonitrile (61 g, 156 mmol) in DMF (300 mL) was added Pd(dppf)Cl₂ (3.43 g, 4.7 mmol), CuI (2.98 g, 15.6 mmol) and Et₃N (23.7 g, 235 mmol), followed by the addition of ethynyl(trimethyl)silane (28 mL, 187 mmol). The resulting mixture was stirred at ambient temperature overnight under a nitrogen atmosphere. The reaction was quenched with water (500 mL) and extracted with ethyl acetate (500 mL×3). The combined organic phase was washed with brine (150 mL×3), dried over anhydrous Na₂SO₄, filtered and concentrated in vacuo. The residue was purified by silica gel column chromatography (eluting with 1:3:3 petroleum ether:EtOAc:DCM) to afford the desired product, 5-[4-amino-2,6-difluoro-3-(2-trimethylsilylethynyl)phenoxy]-2-fluorobenzonitrile (42 g, 74.5%) as a yellow solid. MS (ESI): m/z 361 [M+H]⁺.

### 5-((4,6-Difluoro-1H-indol-5-yl)oxy)-2-fluorobenzonitrile

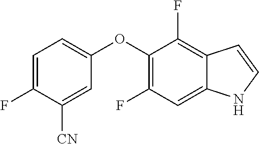 [SMILES: N#Cc1cc(Oc2c(F)cc3[nH]ccc3c2F)ccc1F]

Step E: A mixture of 5-[4-amino-2,6-difluoro-3-(2-trimethylsilylethynyl)phenoxy]-2-fluoro-benzonitrile (28.0 g, 77.7 mmol) and CuI (29.6 g, 155 mmol) in DMF (250 mL) was flushed with argon for 2 minutes and heated for 5 hours at 100° C. under argon. The insoluble materials were removed by suction filtration and the filtrate was diluted with ethyl acetate (1500 mL). The organic phase was washed with brine (300 mL×5), dried over anhydrous Na₂SO₄, filtered, and concentrated to give a residue, which was purified by silica gel column chromatography (eluting with 4:1 petroleum ether:ethyl acetate) to afford the desired product, 5-[(4,6-difluoro-1H-indol-5-yl)oxy]-2-fluorobenzonitrile (18 g, 80%) as a yellow solid. ¹H NMR (500 MHz, DMSO-d₆): δ 11.64 (br, 1H), 7.62 (dd, J=5.0, 3.5 Hz, 1H), 7.51-7.47 (m, 2H), 7.39-7.36 (m, 1H), 7.32 (d, J=10.0 Hz, 1H), 6.58 (t, J=2.0 Hz, 1H) ppm.

### 5-((4,6-Difluoro-1H-indol-5-yl)oxy)-2-fluorobenzoic acid

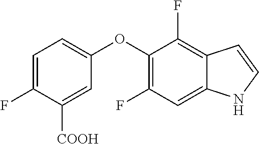 [SMILES: O=C(O)c1cc(Oc2c(F)cc3[nH]ccc3c2F)ccc1F]

Step F: To a solution of 5-[(4,6-difluoro-1H-indol-5-yl)oxy]-2-fluorobenzonitrile (2 g, 6.94 mmol) in ethanol, was added KOH (1.95 g, 3 M, 5 eq). The reaction mixture was stirred at 100° C. for 5 hours. Hydrochloric acid was then added to adjust the reaction pH to 6-7. The resulting mixture was filtered to isolate the desired product, 5-((4,6-difluoro-1H-indol-5-yl)oxy)-2-fluorobenzoic acid (2 g, 94% of yield) as a brown powder. MS (ESI): m/z 308.0 [M+H]⁺.

### Methyl 2-(3-(5,6-dihydro-2H-pyran-2-yl)phenyl)acetate

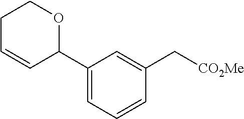 [SMILES: COC(=O)Cc1cccc(C2C=CCCO2)c1]

Step G: To a solution of methyl 2-(3-iodophenyl)acetate (2 g, 7.24 mmol) and 3,4-dihydro-2H-pyran (3 g, 5 eq) in CH₃CN were added Pd(OAc)₂ (65 mg, 0.04 eq), PPh₃ (171 mg, 0.09 eq), and Ag₂CO₃ (4 g, 2 eq). The reaction mixture was stirred overnight at 80° C. EtOAc was added to dilute the mixture. The mixture was filtered and concentrated. The crude was further purified by flash column chromatography on silica, eluting with 10:1 petroleum ether:ethyl acetate to afford the product, methyl 2-(3-(5,6-dihydro-2H-pyran-2-yl)phenyl)acetate (1.1 g, 65%) as colorless oil. MS (ESI): m/z 255.0 [M+Na]⁺.

### Methyl 2-(3-(4-(((benzyloxy)carbonyl)amino)-3-hydroxytetrahydro-2H-pyran-2-yl)phenyl)acetate

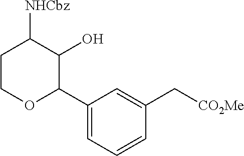 [SMILES: COC(=O)Cc1cccc(C2OCCC(NC(=O)OCc3ccccc3)C2O)c1]

Step H: Osmium tetroxide (175 mg, 0.08 mmol) was added to a solution of benzyloxycarbonylamino 4-chlorobenzoate (3.42 g, 1.3 eq) in acetonitrile (70 mL) and stirred at room temperature for 10 minutes. A solution of methyl 2-(3-(5,6-dihydro-2H-pyran-2-yl)phenyl)acetate (2.0 g, 8.6 mmol) in acetonitrile (10 mL) was added to the carbamate solution followed by the addition of water (8 mL), and the reaction was stirred at room temperature overnight. The reaction was quenched with a saturated aqueous K₂S₂O₅ solution (50 mL) and stirred for a further 5 min. Water (100 mL) was added, and the resulting mixture was extracted with ethyl acetate (80 mL×2). The organic layers were combined, washed with saturated aqueous NaHCO₃ (80 mL×2), brine, and dried over MgSO₄. The solvent was evaporated under reduced pressure. The residue was purified by flash column chromatography on silica gel to yield the product, methyl 2-(3-(4-(((benzyloxy)carbonyl)amino)-3-hydroxytetrahydro-2H-pyran-2-yl)phenyl)acetate (2.1 g, 61%) as a dark oil. MS (ESI): m/z 422.0 [M+Na]⁺.

### Methyl 2-(3-(4-amino-3-hydroxytetrahydro-2H-pyran-2-yl)phenyl)acetate

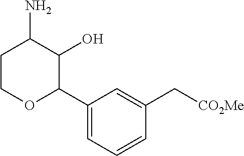 [SMILES: COC(=O)Cc1cccc(C2OCCC(N)C2O)c1]

Step I: To a solution of methyl 2-(3-(4-(((benzyloxy)carbonyl)amino)-3-hydroxytetrahydro-2H-pyran-2-yl)phenyl)acetate (500 mg, 1.25 mmol) in methanol, a catalytic amount of Pd/C (10 wt. %) was added. The reaction was stirred under the H₂ atmosphere overnight. The resulting mixture was filtered and concentrated to get the product, methyl 2-(3-(4-amino-3-hydroxytetrahydro-2H-pyran-2-yl)phenyl)acetate (240 mg, 72%) as a light yellow oil. MS (ESI): m/z 266.0 [M+H]⁺.

### Methyl 2-(3-(4-(5-((4,6-difluoro-1H-indol-5-yl)oxy)-2-fluorobenzamido)-3-hydroxytetrahydro-2H-pyran-2-yl)phenyl)acetate

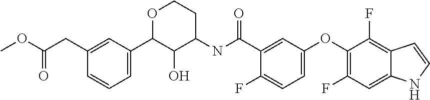 [SMILES: COC(=O)Cc1cccc(C2OCCC(NC(=O)c3cc(Oc4c(F)cc5[nH]ccc5c4F)ccc3F)C2O)c1]

Step J: To a solution of 5-((4,6-difluoro-1H-indol-5-yl)oxy)-2-fluorobenzoic acid (product from Step F, 250 mg, 0.814 mmol) and methyl 2-(3-(4-amino-3-hydroxytetrahydro-2H-pyran-2-yl)phenyl)acetate (216 mg, 1 eq) in DMF (10 mL) were added HATU (371 mg, 1.2 eq) and DIPEA (0.418 ml, 3 eq). The reaction mixture was stirred at room temperature for 2 hours. Water was added to quench the reaction, and the reaction mixture was extracted thoroughly with EtOAc. The organic phases were combined and washed with brine, and then concentrated. The crude product was purified by flash column chromatography on silica gel, eluting with 3:1 petroleum ether:EtOAc to obtain the product, methyl 2-(3-(4-(5-((4,6-difluoro-1H-indol-5-yl)oxy)-2-fluorobenzamido)-3-hydroxytetrahydro-2H-pyran-2-yl)phenyl)acetate (420 mg, 93%) as a yellow powder. MS (ESI): m/z 555.0 [M+H]⁺.

### Methyl 2-(3-(4-(5-((4,6-difluoro-1H-indol-5-yl)oxy)-2-fluorobenzamido)-3-oxotetrahydro-2H-pyran-2-yl)phenyl)acetate

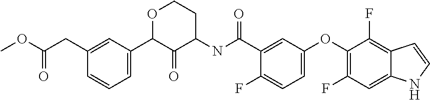 [SMILES: COC(=O)Cc1cccc(C2OCCC(NC(=O)c3cc(Oc4c(F)cc5[nH]ccc5c4F)ccc3F)C2=O)c1]

Step K: To a solution of methyl 2-(3-(4-(5-((4,6-difluoro-1H-indol-5-yl)oxy)-2-fluorobenzamido)-3-hydroxytetrahydro-2H-pyran-2-yl)phenyl)acetate (1300 mg, 0.234 mmol) in DMSO (50 mL), 2-iodoxybenzoic acid (3.28 g, 5 eq) was added. The reaction mixture was stirred at 50° C. overnight. Saturated aqueous Na₂CO₃ was added to quench the reaction. The solids were isolated by suction filtration and washed with water. The solid residue was dissolved in EtOAc and washed with water, brine, and concentrated. The crude was further purified by flash column chromatography on silica gel, eluting with 1:2 petroleum ether:EtOAc to obtain the product, methyl 2-(3-(4-(5-((4,6-difluoro-1H-indol-5-yl)oxy)-2-fluorobenzamido)-3-oxotetrahydro-2H-pyran-2-yl)phenyl)acetate (500 mg, 38%) as yellow powder. MS (ESI): m/z 553.0 [M+H]⁺.

### Methyl 2-(3-(2-(5-((4,6-difluoro-1H-indol-5-yl)oxy)-2-fluorophenyl)-1,4,6,7-tetrahydropyrano[3,4-d]imidazol-4-yl)phenyl)acetate

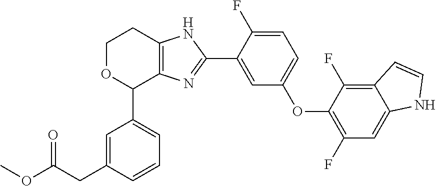 [SMILES: COC(=O)Cc1cccc(C2OCCc3[nH]c(-c4cc(Oc5c(F)cc6[nH]ccc6c5F)ccc4F)nc32)c1]

Step L: To a solution of methyl 2-(3-(4-(5-((4,6-difluoro-1H-indol-5-yl)oxy)-2-fluorobenzamido)-3-oxotetrahydro-2H-pyran-2-yl)phenyl)acetate (100 mg, 0.18 mmol) in toluene, ammonium acetate (140 mg, 10 eq) was added. The reaction vessel was sealed, and the reaction mixture stirred at 110° C. for 4 hours. The solvent was removed under reduced pressure and the mixture was extracted with EtOAc. The organic phases were combined and washed with brine, dried over Na₂SO₄, and concentrated. The crude was further purified by flash column chromatography on silica gel, eluting with 1:2 petroleum ether:EtOAc to get the product, methyl 2-(3-(2-(5-((4,6-difluoro-1H-indol-5-yl)oxy)-2-fluorophenyl)-1,4,6,7-tetrahydropyrano[3,4-d]imidazol-4-yl)phenyl)acetate (8 mg, 8%) as a yellow powder. MS (ESI): m/z 534.0 [M+H]⁺.

### 2-(3-(2-(5-((4,6-Difluoro-1H-indol-5-yl)oxy)-2-fluorophenyl)-1,4,6,7-tetrahydropyrano[3,4-d]imidazol-4-yl)phenyl)acetic acid

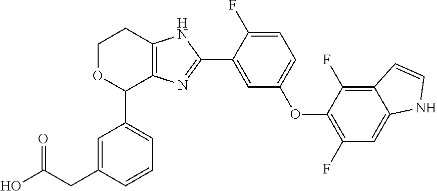 [SMILES: O=C(O)Cc1cccc(C2OCCc3[nH]c(-c4cc(Oc5c(F)cc6[nH]ccc6c5F)ccc4F)nc32)c1]

Step M: To a solution of methyl 2-(3-(2-(5-((4,6-difluoro-1H-indol-5-yl)oxy)-2-fluorophenyl)-1,4,6,7-tetrahydropyrano[3,4-d]imidazol-4-yl)phenyl)acetate (8 mg, 0.014 mmol) in 3:1 THF:MeOH, LiOH (2 eq) was added. The reaction mixture was stirred at room temperature for 2 hours. Water was added and the volatiles were removed under reduced pressure. The pH was adjusted to ~6-7, and the mixture extracted with EtOAc. The organic phases were combined and washed with saturated aqueous NaHCO₃, brine, dried over Na₂SO₄ and concentrated. The crude was purified by prep-HPLC to yield the product, 2-(3-(2-(5-((4,6-difluoro-1H-indol-5-yl)oxy)-2-fluorophenyl)-1,4,6,7-tetrahydropyrano[3,4-d]imidazol-4-yl)phenyl)acetic acid (4.2 mg, 54%) as a grey powder. MS (ESI): m/z 520.0 [M+H]⁺. ¹H NMR (400 MHz, CD₃OD): δ 7.43 (dd, J=6.0, 3.2 Hz, 1H), 7.29-7.26 (m, 4H), 7.20-7.17 (m, 2H), 7.11-7.06 (m, 1H), 6.94 (dt, J=7.2, 3.6 Hz, 1H), 6.52 (dd, J=3.2, 0.8 Hz, 1H), 5.70 (s, 1H), 4.06-4.01 (m, 1H), 3.91-3.85 (m, 1H), 3.55 (s, 2H), 2.90-2.83 (m, 1H), 2.80-2.76 (m, 1H) ppm.

### Example 3. Synthesis of (R)-3-(3-(2-(5-((4,6-difluoro-1H-indol-5-yl)oxy)-2-fluorophenyl)-4,5,6,7-tetrahydro-3H-imidazo[4,5-c]pyridin-4-yl)-2-fluorophenyl)propanoic acid

### Example 4. Synthesis of (S)-3-(3-(2-(5-((4,6-difluoro-1H-indol-5-yl)oxy)-2-fluorophenyl)-4,5,6,7-tetrahydro-3H-imidazo[4,5-c]pyridin-4-yl)-2-fluorophenyl)propanoic acid

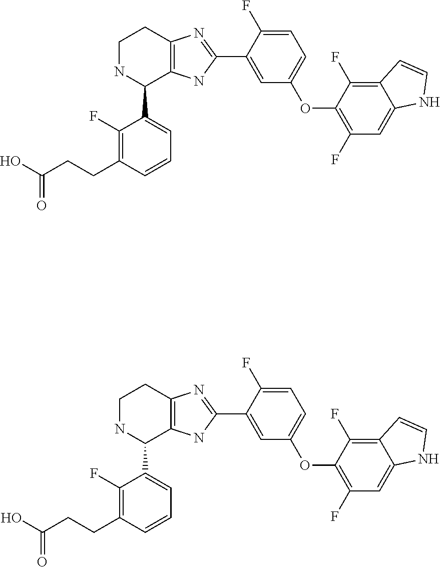 [SMILES: O=C(O)CCc1cccc([C@H]2NCCc3nc(-c4cc(Oc5c(F)cc6[nH]ccc6c5F)ccc4F)[nH]c32)c1F ; O=C(O)CCc1cccc([C@@H]2NCCc3nc(-c4cc(Oc5c(F)cc6[nH]ccc6c5F)ccc4F)[nH]c32)c1F]

### 5-((4,6-Difluoro-1H-indol-5-yl)oxy)-2-fluorobenzimidamide

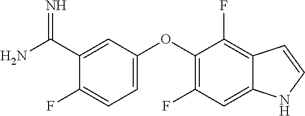 [SMILES: N=C(N)c1cc(Oc2c(F)cc3[nH]ccc3c2F)ccc1F]

Step A: To a solution of 5-[(4,6-difluoro-1H-indol-5-yl)oxy]-2-fluoro-benzonitrile (Step E, Example 2) (440 mg, 1.53 mmol) in THE (10 mL) was added at 0° C. under a nitrogen atmosphere a solution of LHMDS (1 M in THF, 6.12 mL). The resulting mixture was stirred at 25° C. overnight. The reaction was quenched with water (0.2 mL) and diluted with ethyl acetate (30 mL). The mixture was washed with brine (10 mL×2), dried over anhydrous Na₂SO₄, filtered, and concentrated in vacuo to give the crude title compound (500 mg), which was used directly in the next step. MS (ESI): m/z 306.1 [M+H]⁺.

### 2-(2-(Oxiran-2-yl)ethyl)isoindoline-1,3-dione

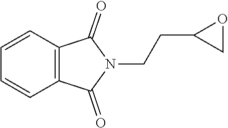 [SMILES: O=C1c2ccccc2C(=O)N1CCC1CO1]

Step B: A solution of phthalimide potassium salt (1.47 g, 7.9 mmol) in anhydrous DMF (17 mL) was stirred at room temperature for 10 minutes under an argon atmosphere, then a solution of 2-(2-bromoethyl)oxirane (1.0 g, 6.62 mmol) in anhydrous DMF (3 mL) was added dropwise. The reaction mixture was stirred at room temperature for 20 hours. The solids were filtered out and washed with EtOAc (100 mL). The combined filtrates were washed with water (30 mL), brine (50 mL×5), dried over anhydrous magnesium sulfate, filtered, and concentrated to afford the desired product, 2-(2-(oxiran-2-yl)ethyl)isoindoline-1,3-dione (1.44 g, 96%) as an off-white solid. MS (ESI): m/z 218 [M+H]⁺.

### 2-(4-Chloro-3-hydroxy-butyl)isoindoline-1, 3-dione

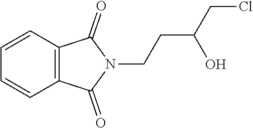 [SMILES: O=C1c2ccccc2C(=O)N1CCC(O)CCl]

Step C: A solution of 2-[2-(oxiran-2-yl)ethyl]isoindoline-1,3-dione (1.44 g, 6.36 mmol) in CHCl₃ (20 mL) was cooled to 0° C. and 12 M aqueous HCl (10.4 mL, 19.5 mmol) was added. The reaction was stirred at 0° C. for 30 minutes then water (100 mL) was added, the mixture extracted with DCM (150 mL×2), the extracts washed with brine (50 mL×2), dried over anhydrous MgSO₄, and concentrated under reduced pressure to afford the desired product, 2-(4-chloro-3-hydroxy-butyl)isoindoline-1,3-dione (1.6 g, 87%) as a white solid. MS (ESI): m/z 254 [M+H]⁺.

### 2-(4-Chloro-3-oxo-butyl)isoindoline-1,3-dione

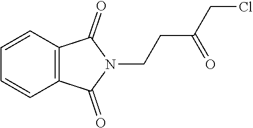 [SMILES: O=C(CCl)CCN1C(=O)c2ccccc2C1=O]

Step D: A mixture of 2-(4-chloro-3-hydroxy-butyl)isoindoline-1,3-dione (1.6 g, 6.3 mmol) and Dess-Martin periodinane (4.01 g, 9.46 mmol) in DCM (40 mL) was stirred at room temperature for 5 hours under N₂. 150 mL saturated aqueous NaHCO₃ was added, and the resulting mixture was extracted with DCM (60 mL×3). The combined organic layers were concentrated, and the residue purified by flash column chromatography on silica gel, eluting with 0-20% acetone in petroleum ether, to afford the desired product, 2-(4-chloro-3-oxo-butyl)isoindoline-1,3-dione (1.2 g, 73%) as a white solid. MS (ESI): m/z 252 [M+H]⁺.

### 2-(2-(5-((4,6-Difluoro-1H-indol-5-yl)oxy)-2-fluorophenyl)-1H-imidazol-5-yl)ethan-1-amine

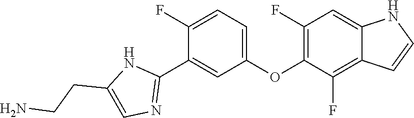 [SMILES: NCCc1cnc(-c2cc(Oc3c(F)cc4[nH]ccc4c3F)ccc2F)[nH]1]

Step E: A mixture of 2-(4-chloro-3-oxobutyl)isoindoline-1,3-dione (1.2 g, 4.77 mmol), 5-[(4,6-difluoro-1H-indol-5-yl)oxy]-2-fluoro-benzamidine (product from Step A, 1.46 g, 4.77 mmol) and sodium bicarbonate (1.2 g, 14.2 mmol) in DMF (15 mL) was stirred at room temperature for 30 minutes, then warmed to 70° C. and stirred at that temperature overnight. After cooling to room temperature, 200 mL of EtOAc was added and the resulting mixture was washed with brine (100 mL×5). The organic layer was concentrated. The residue was purified by flash column chromatography on silica gel, eluting with 0-30% acetone in petroleum ether, to afford the desired product, 2-[2-[2-[5-[(4,6-difluoro-1H-indol-5-yl)oxy]-2-fluoro-phenyl]-1H-imidazol-5-yl]ethyl]isoindoline-1,3-dione (1.6 g, 64.8%) as a light yellow solid. MS (ESI): m/z 503 [M+H]⁺.

Step F: To a solution of 2-[2-[2-[5-[(4,6-difluoro-1H-indol-5-yl)oxy]-2-fluoro-phenyl]-1H-imidazol-5-yl]ethyl]isoindoline-1,3-dione (1.6 g, 3.18 mmol) in EtOH (30 mL) was added hydrazine hydrate (488 mg, 9.6 mmol) and the resulting mixture was stirred at 50° C. for 2 hours. The reaction mixture was concentrated under reduced pressure. The residue was purified by silica gel column chromatography, eluting with a 100:1 to 4:1 DCM:MeOH gradient (MeOH contains 0.5% concentrated aqueous ammonia), to afford the desired product, 2-[2-[5-[(4,6-difluoro-1H-indol-5-yl)oxy]-2-fluoro-phenyl]-1H-imidazol-5-yl]ethanamine (980 mg, 83%) as a yellow solid. MS (ESI): m/z 373.6 [M+H]⁺.

### Ethyl (E)-3-(2-fluoro-3-formylphenyl)acrylate

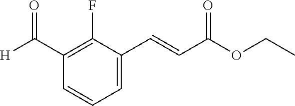 [SMILES: CCOC(=O)/C=C/c1cccc(C=O)c1F]

Step G: To a solution of 3-bromo-2-fluoro-benzaldehyde (300 mg, 1.48 mmol), tris-o-tolylphosphane (90 mg, 0.3 mmol), triethylamine (449 mg, 4.4 mmol) and ethyl prop-2-enoate (444 mg, 4.4 mmol) in DMF (1.5 mL) was added Pd(OAc)₂ (33 mg, 0.15 mmol). The reaction mixture was stirred overnight at 120° C. under argon. To the cooled reaction was added H₂O (50 mL) and the resulting mixture was extracted with EtOAc (50 mL×3). The combined organic layer was dried over sodium sulfate, filtered, and evaporated under reduced pressure. The residue was purified by flash column chromatography on silica gel, eluting with 0-15% acetone in petroleum ether, to afford the desired product, ethyl (E)-3-(2-fluoro-3-formyl-phenyl)prop-2-enoate (210 mg, 60%) as a yellow solid. MS (ESI): m/z 223 [M+H]⁺.

### (E)-3-(3-(2-(5-((4,6-Difluoro-1H-indol-5-yl)oxy)-2-fluorophenyl)-4,5,6,7-tetrahydro-3H-imidazo[4,5-c]pyridin-4-yl)-2-fluorophenyl)acrylic acid

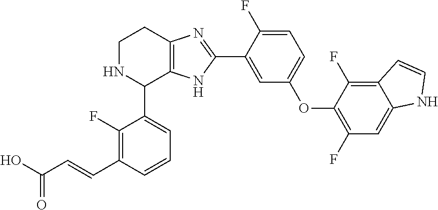 [SMILES: O=C(O)/C=C/c1cccc(C2NCCc3nc(-c4cc(Oc5c(F)cc6[nH]ccc6c5F)ccc4F)[nH]c32)c1F]

Step H: To a mixture of 2-[2-[5-[(4,6-difluoro-1H-indol-5-yl)oxy]-2-fluoro-phenyl]-1H-imidazol-4-yl]ethanamine (500 mg, 1.34 mmol), ethyl (E)-3-(2-fluoro-3-formyl-phenyl)prop-2-enoate (358 mg, 1.61 mmol) in MeOH (8 mL) was slowly added, under nitrogen at 0° C., a solution of NaOH (548 mg, 13.8 mmol) in water (4 mL). The reaction mixture was stirred at room temperature for 0.5 hours, then warmed to 70° C., and stirred at that temperature for 5 hours. The reaction mixture was cooled, and the pH adjusted to 7-8 with 1M hydrochloric acid. The mixture was concentrated and the residue was purified by flash column chromatography on C18 silica, eluting with 0-60% MeOH in 10 mM aqueous NH₄HCO₃, to afford the partially purified desired product (E)-3-(3-(2-(5-((4,6-difluoro-1H-indol-5-yl)oxy)-2-fluorophenyl)-4,5,6,7-tetrahydro-3H-imidazo[4,5-c]pyridin-4-yl)-2-fluorophenyl)acrylic acid (700 mg) as a white solid. MS (ESI): m/z 549 [M+H]⁺.

### 3-(3-(2-(5-((4,6-Difluoro-1H-indol-5-yl)oxy)-2-fluorophenyl)-4,5,6,7-tetrahydro-3H-imidazo[4,5-c]pyridin-4-yl)-2-fluorophenyl)propanoic acid

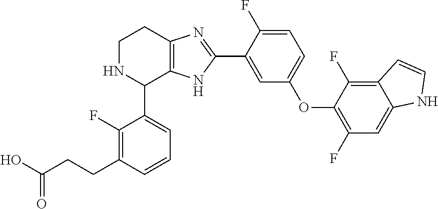 [SMILES: O=C(O)CCc1cccc(C2NCCc3nc(-c4cc(Oc5c(F)cc6[nH]ccc6c5F)ccc4F)[nH]c32)c1F]

Step I: To a solution of crude (E)-3-[3-[2-[5-[(4,6-difluoro-1H-indol-5-yl)oxy]-2-fluoro-phenyl]-4,5,6,7-tetrahydro-3H-imidazo[4,5-c]pyridin-4-yl]-2-fluoro-phenyl]prop-2-enoic acid (700 mg, 1.28 mmol) in THE (50 mL) was added Pd/C (10 wt. %, 250 mg). The mixture was stirred at room temperature overnight under H₂. The reaction mixture was filtered through Celite, and the filtrate was concentrated to give 400 mg of crude product.

### (R)-3-(3-(2-(5-((4,6-Difluoro-1H-indol-5-yl)oxy)-2-fluorophenyl)-4,5,6,7-tetrahydro-3H-imidazo[4,5-c]pyridin-4-yl)-2-fluorophenyl)propanoic acid and (S)-3-(3-(2-(5-((4,6-difluoro-1H-indol-5-yl)oxy)-2-fluorophenyl)-4,5,6,7-tetrahydro-3H-imidazo[4,5-c]pyridin-4-yl)-2-fluorophenyl)propanoic acid

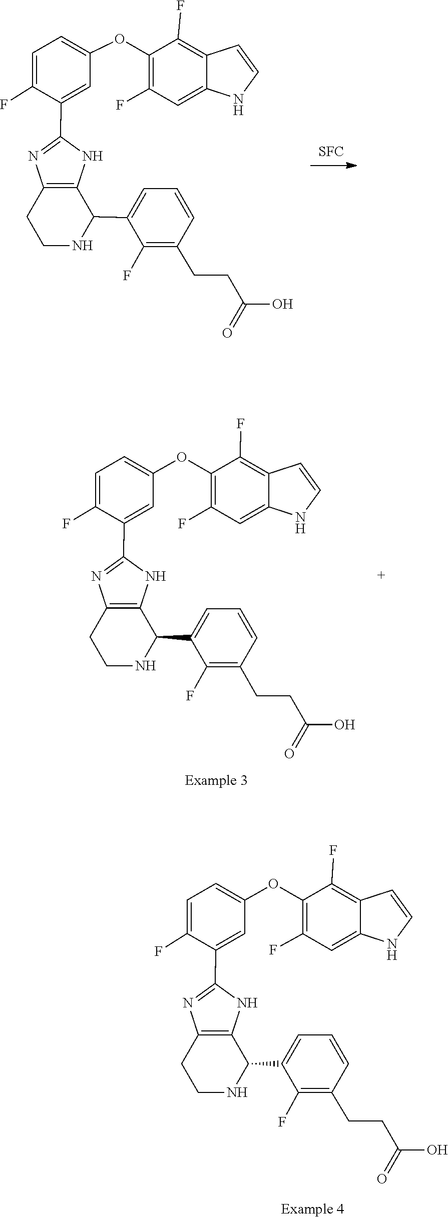 [SMILES: O=C(O)CCc1cccc(C2NCCc3nc(-c4cc(Oc5c(F)cc6[nH]ccc6c5F)ccc4F)[nH]c32)c1F ; "SFC" ; O=C(O)CCc1cccc([C@H]2NCCc3nc(-c4cc(Oc5c(F)cc6[nH]ccc6c5F)ccc4F)[nH]c32)c1F ; O=C(O)CCc1cccc([C@@H]2NCCc3nc(-c4cc(Oc5c(F)cc6[nH]ccc6c5F)ccc4F)[nH]c32)c1F]

Step J: The above crude product mixture (400 mg) was subjected to chiral separation by SFC to provide a crude, faster eluting component, and a crude, slower eluting component. The absolute configuration of the faster eluting component was arbitrarily assigned as (R)-3-(3-(2-(5-((4,6-difluoro-1H-indol-5-yl)oxy)-2-fluorophenyl)-4,5,6,7-tetrahydro-3H-imidazo[4,5-c]pyridin-4-yl)-2-fluorophenyl)propanoic acid (Example 3). The absolute configuration of the slower eluting component was therefore assigned as (S)-3-(3-(2-(5-((4,6-difluoro-1H-indol-5-yl)oxy)-2-fluorophenyl)-4,5,6,7-tetrahydro-3H-imidazo[4,5-c]pyridin-4-yl)-2-fluorophenyl)propanoic acid (Example 4). The crude products were additionally purified by prep-HPLC (Mobile Phase: A=10 mM NH₄HCO₃ in water, B=acetonitrile; Gradient: 25-55% B in 10 min, stop at 18 min; Flow rate: 30 mL/min; Retention time, 8.21 min; column: Welch XB-C18 21.2*250 mm, 10 μm) to afford 3-[3-[(4R)-2-[5-[(4,6-difluoro-1H-indol-5-yl)oxy]-2-fluoro-phenyl]-4,5,6,7-tetrahydro-3H-imidazo[4,5-c]pyridin-4-yl]-2-fluoro-phenyl]propanoic acid (87.8 mg, 12% over 3 steps) as a white solid and 3-[3-[(4S)-2-[5-[(4,6-difluoro-1H-indol-5-yl)oxy|-2-fluoro-phenyl|-4,5,6,7-tetrahydro-3H-imidazo|4,5-c|pyridin-4-yl]-2-fluoro-phenyl]propanoic acid (58.1 mg, 7.9% over 3 steps) as a white solid.

### Chiral Separation Conditions:

Instrument: SFC-80 (Thar, Waters)
Column: SSWHELK 20*250 mm, 10 μm (Daicel)
Column temperature: 35° C.
Mobile phase: 30/70 CO₂/EtOH (containing 0.5% ethanolamine)
Flow rate: 80 g/min
Back pressure: 100 bar
Detection wavelength: 214 nm
Cycle time: 5 min
Sample solution: 400 mg dissolved in 40 ml methanol; Injection volume: 4.5 ml

3-[3-[(4R)-2-[5-[(4,6-difluoro-1H-indol-5-yl)oxy]-2-fluoro-phenyl]-4,5,6,7-tetrahydro-3H-imidazo[4,5-c]pyridin-4-yl]-2-fluoro-phenyl]propanoic acid—MS (ESI): m/z 551 [M+H]⁺. ¹H NMR (400 MHz, CD₃OD): δ 7.41 (q, J=2.8 Hz, 1H), 7.34 (t, J=6.8 Hz, 1H), 7.29 (d, J=3.6 Hz, 1H), 7.21 (t, J=9.6 Hz, 1H), 7.14-6.99 (m, 3H), 6.89 (t, J=6.8 Hz, 1H), 6.53 (d, J=3.2 Hz, 1H), 5.74 (s, 1H), 3.31 (t, J=7.6 Hz, 2H), 3.03-2.97 (m, 4H), 2.56 (t, J=7.6 Hz, 2H) ppm.

3-[3-[(4S)-2-[5-[(4,6-difluoro-1H-indol-5-yl)oxy]-2-fluoro-phenyl]-4,5,6,7-tetrahydro-3H-imidazo[4,5-c]pyridin-4-yl]-2-fluoro-phenyl]propanoic acid—MS (ESI): m/z 551 [M+H]⁺. ¹H NMR (400 MHz, CD₃OD) δ 7.41 (q, J=2.8 Hz, 1H), 7.34 (t, J=6.8 Hz, 1H), 7.29 (d, J=3.6 Hz, 1H), 7.21 (t, J=9.6 Hz, 1H), 7.14-6.99 (m, 3H), 6.89 (t, J=6.8 Hz, 1H), 6.53 (d, J=3.2 Hz, 1H), 5.74 (s, 1H), 3.31 (t, J=7.6 Hz, 2H), 3.03-2.97 (m, 4H), 2.56 (t, J=7.6 Hz, 2H) ppm.

### Example 5. Synthesis of 3-[3-[(4R)-2-[5-[(4,6-difluoro-1H-indol-5-yl)oxy]-2-fluoro-phenyl]-5-methyl-3,4,6,7-tetrahydroimidazo[4,5-c]pyridin-4-yl]-2-fluoro-phenyl]propanoic acid

### Example 6. Synthesis 3-[3-[(4S)-2-[5-[(4,6-difluoro-1H-indol-5-yl)oxy]-2-fluoro-phenyl]-5-methyl-3,4,6,7-tetrahydroimidazo[4,5-c]pyridin-4-yl]-2-fluoro-phenyl]propanoic acid

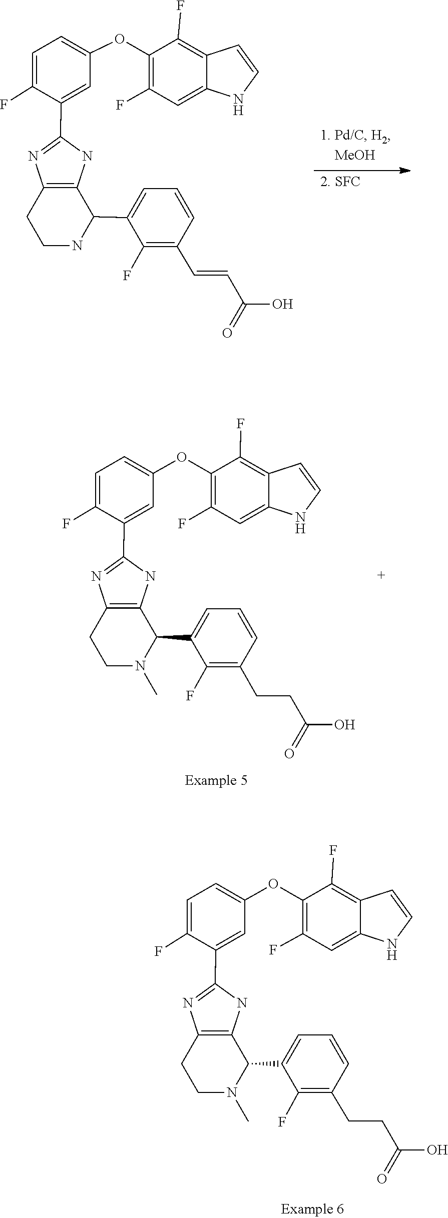 [SMILES: O=C(O)/C=C/c1cccc(C2NCCc3nc(-c4cc(Oc5c(F)cc6[nH]ccc6c5F)ccc4F)[nH]c32)c1F ; CN1CCc2nc(-c3cc(Oc4c(F)cc5[nH]ccc5c4F)ccc3F)[nH]c2[C@H]1c1cccc(CCC(=O)O)c1F ; CN1CCc2nc(-c3cc(Oc4c(F)cc5[nH]ccc5c4F)ccc3F)[nH]c2[C@@H]1c1cccc(CCC(=O)O)c1F]

To a solution of (E)-3-[3-[2-[5-[(4,6-difluoro-1H-indol-5-yl)oxy]-2-fluoro-phenyl]-4,5,6,7-tetrahydro-3H-imidazo[4,5-c]pyridin-4-yl]-2-fluoro-phenyl]prop-2-enoic acid (310 mg, 0.56 mmol) in MeOH (100 mL) was added Pd/C (0.6 g, 10 wt. %). The mixture was stirred at room temperature for 72 hours under H₂. The mixture was filtered through Celite, and the filtrate was concentrated to give the crude product. The crude product was purified by SFC to afford a faster eluting component, and a slower eluting component. The absolute configuration of the faster eluting component was arbitrarily assigned as 3-[3-[(4R)-2-[5-[(4,6-difluoro-1H-indol-5-yl)oxy]-2-fluoro-phenyl]-5-methyl-3,4,6,7-tetrahydroimidazo[4,5-c]pyridin-4-yl]-2-fluoro-phenyl]propanoic acid (33.8 mg, 10.6%, a white solid) and that of the slower eluting component as 3-[3-[(4S)-2-[5-[(4,6-difluoro-1H-indol-5-yl)oxy]-2-fluoro-phenyl]-5-methyl-3,4,6,7-tetrahydroimidazo[4,5-c]pyridin-4-yl]-2-fluoro-phenyl]propanoic acid (26.6 mg, 8.3%, a white solid).

### Chiral Separation Conditions:

Instrument: SFC-80 (Thar, Waters)
Column: IE 20*250 mm, 10 μm (Daicel)
Column temperature: 35° C.
Mobile phase: 50/50 CO₂/methanol (0.2% ammonia in methanol)
Flow rate: 80 g/minutes; Back pressure: 100 bar
Detection wavelength: 214 nm
Cycle time: 4 min
Sample solution: 130 mg dissolved in 15 ml methanol; Injection volume: 1.5 ml

3-[3-[(4R)-2-[5-[(4,6-difluoro-1H-indol-5-yl)oxy]-2-fluoro-phenyl]-5-methyl-3,4,6,7-tetrahydroimidazo[4,5-c]pyridin-4-yl]-2-fluoro-phenyl]propanoic acid (Example 5)—MS (ESI): m/z 565 [M+H]⁺. ¹H NMR (400 MHz, CD₃OD) δ 7.38 (q, J=2.8 Hz, 1H), 7.29 (d, J=3.2 Hz, 1H), 7.27 (t, J=6.8 Hz, 1H), 7.18 (t, J=9.6 Hz, 1H), 7.11 (d, J=10.0 Hz, 1H), 7.04 (t, J=7.6 Hz, 1H), 6.98-6.94 (m, 1H), 6.86 (t, J=7.2 Hz, 1H), 6.53 (d, J=2.4 Hz, 1H), 5.09 (s, 1H), 3.17-3.12 (m, 1H), 3.00 (t, J=7.6 Hz, 2H), 2.90-2.82 (m, 3H), 2.61 (t, J=7.6 Hz, 2H), 2.44 (s, 3H) ppm.

3-[3-[(4S)-2-[5-[(4,6-difluoro-1H-indol-5-yl)oxy]-2-fluoro-phenyl]-5-methyl-3,4,6,7-tetrahydroimidazo[4,5-c]pyridin-4-yl]-2-fluoro-phenyl]propanoic acid (Example 5)—MS (ESI): m/z 565 [M+H]⁺. ¹H NMR (400 MHz, CD₃OD) δ 7.38 (q, J=2.8 Hz, 1H), 7.29 (d, J=3.2 Hz, 1H), 7.27 (t, J=6.8 Hz, 1H), 7.18 (t, J=9.6 Hz, 1H), 7.11 (d, J=10.0 Hz, 1H), 7.04 (t, J=7.6 Hz, 1H), 6.98-6.94 (m, 1H), 6.86 (t, J=7.2 Hz, 1H), 6.53 (d, J=2.4 Hz, 1H), 5.09 (s, 1H), 3.17-3.12 (m, 1H), 3.00 (t, J=7.6 Hz, 2H), 2.90-2.82 (m, 3H), 2.61 (t, J=7.6 Hz, 2H), 2.44 (s, 3H) ppm.

### Example 7. Synthesis of 3-[2-fluoro-3-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]phenyl]-2-methyl-propanoic acid

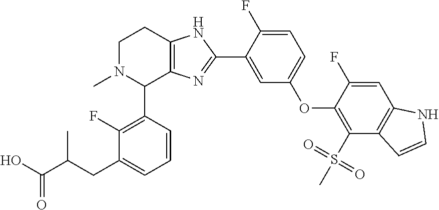 [SMILES: CC(Cc1cccc(C2c3nc(-c4cc(Oc5c(F)cc6[nH]ccc6c5S(C)(=O)=O)ccc4F)[nH]c3CCN2C)c1F)C(=O)O]

### 2-Fluoro-5-[6-fluoro-4-methylsulfonyl-1-(p-tolylsulfonyl)indol-5-yl]oxy-N-hydroxy-benzamidine

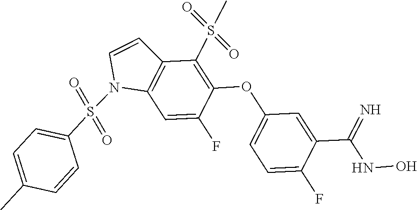 [SMILES: Cc1ccc(S(=O)(=O)n2ccc3c(S(C)(=O)=O)c(Oc4ccc(F)c(C(=N)NO)c4)c(F)cc32)cc1]

Step A: To a stirred solution of 2-fluoro-5-[6-fluoro-4-methylsulfonyl-1-(p-tolylsulfonyl)indol-5-yl]oxy-benzonitrile (WO2023034992) (1.00 eq, 100 mg, 0.199 mmol) in methanol (2 mL) and THE (0.2 mL) was added hydroxylamine hydrochloride (4.00 eq, 55 mg, 0.796 mmol) and TEA (5.00 eq, 0.14 mL, 0.995 mmol) at rt. The reaction mixture was stirred at rt for 16 h and reaction progress was followed by TLC and LC-MS. The reaction mixture was poured into water and the solid precipitate was collected by filtration, the solid was washed with water and dried under reduced pressure to afford 2-fluoro-5-[6-fluoro-4-methylsulfonyl-1-(p-tolylsulfonyl)indol-5-yl]oxy-N-hydroxy-benzamidine (80 mg, 0.149 mmol, 75% yield) as a white solid. MS (ESI): m/z 536 [M+H]⁺.

### [[2-Fluoro-5-[6-fluoro-4-methylsulfonyl-1-(p-tolylsulfonyl)indol-5-yl]oxy-benzenecarboximidoyl]amino]acetate

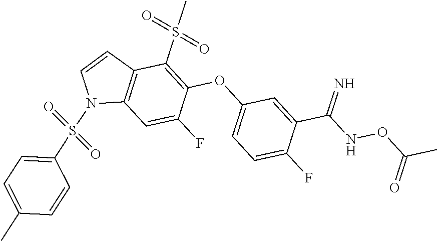 [SMILES: CC(=O)ONC(=N)c1cc(Oc2c(F)cc3c(ccn3S(=O)(=O)c3ccc(C)cc3)c2S(C)(=O)=O)ccc1F]

Step B: To a stirred solution of 2-fluoro-5-[6-fluoro-4-methylsulfonyl-1-(p-tolylsulfonyl)indol-5-yl]oxy-N-hydroxy-benzamidine (1.00 eq, 80 mg, 0.150 mmol) in acetic acid (0.80 mL) was added at 20° C. acetic anhydride (1.10 eq, 0.016 mL, 0.165 mmol). The reaction was stirred for 30 min at 20° C., diluted with DCM, and neutralized with a saturated aqueous NaHCO₃ solution. The separated organic phase was dried over Na₂SO₄, filtered, and concentrated. The crude was triturated with 10% EtOAc in hexane to afford [[2-fluoro-5-[6-fluoro-4-methylsulfonyl-1-(p-tolylsulfonyl)indol-5-yl]oxy-benzenecarboximidoyl]amino]acetate (40 mg, 0.0693 mmol, 46% yield) as a white solid. MS (ESI): m/z 578 [M+H]⁺.

### 2-Fluoro-5-[6-fluoro-4-methylsulfonyl-]-(p-tolylsulfonyl)indol-5-yl]oxy-benzamidine

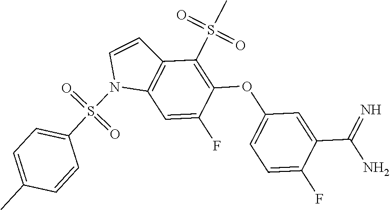 [SMILES: Cc1ccc(S(=O)(=O)n2ccc3c(S(C)(=O)=O)c(Oc4ccc(F)c(C(=N)N)c4)c(F)cc32)cc1]

Step C: To a stirred solution of [[2-fluoro-5-[6-fluoro-4-methylsulfonyl-1-(p-tolylsulfonyl)indol-5-yl]oxy-benzenecarboximidoyl]amino]acetate (1.00 eq, 40 mg, 0.0693 mmol) in methanol (1 mL) was added Pd/C (10% wt., 15 mg). The resulting suspension was stirred under a hydrogen atmosphere for 1 hour at ambient temperature. The reaction was filtered through a Celite pad, and the pad was rinsed with methanol. The combined filtrates were concentrated. The crude was washed with MTB, and dried under reduced pressure to afford 2-fluoro-5-[6-fluoro-4-methylsulfonyl-1-(p-tolylsulfonyl)indol-5-yl]oxy-benzamidine (30 mg, 0.0577 mmol, 83% yield) as an off white solid. MS (ESI): m/z 520 [M+H]⁺.

### 2-[2-[2-[2-Fluoro-5-[6-fluoro-4-methylsulfonyl-]-(p-tolylsulfonyl)indol-5-yl]oxy-phenyl]-1H-imidazol-5-yl]ethyl]isoindoline-1,3-dione

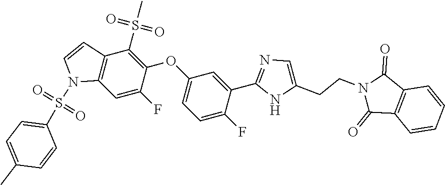 [SMILES: Cc1ccc(S(=O)(=O)n2ccc3c(S(C)(=O)=O)c(Oc4ccc(F)c(-c5ncc(CCN6C(=O)c7ccccc7C6=O)[nH]5)c4)c(F)cc32)cc1]

Step D: To a stirred solution of 2-fluoro-5-[6-fluoro-4-methylsulfonyl-1-(p-tolylsulfonyl)indol-5-yl]oxy-benzamidine (1.00 eq, 2.00 g, 3.85 mmol) and 2-(4-chloro-3-oxo-butyl)isoindoline-1,3-dione (1.50 eq, 1.45 g, 5.77 mmol) in DMF (20 mL) was added at rt sodium bicarbonate (3.00 eq, 0.97 g, 11.5 mmol). The resulting mixture was stirred at RT for 30 min, then at 70° C. for 16 h. The reaction was cooled to RT, diluted with water, and extracted with ethyl acetate. The separated organic phase was dried over Na₂SO₄, filtered, and concentrated. The crude was purified by flash column chromatography on silica gel, eluting with 40-50% EtOAc in hexane, to afford 2-[2-[2-[2-fluoro-5-[6-fluoro-4-methylsulfonyl-1-(p-tolylsulfonyl)indol-5-yl]oxy-phenyl]-1H-imidazol-5-yl]ethyl]isoindoline-1,3-dione (2.10 g, 2.93 mmol, 76% yield) as an off-white solid. MS (ESI): m/z 717 [M+H]⁺.

### 2-[2-[2-Fluoro-5-[6-fluoro-4-methylsulfonyl-]-(p-tolylsulfonyl)indol-5-yl]oxy-phenyl]-1H-imidazol-4-yl]ethanamine

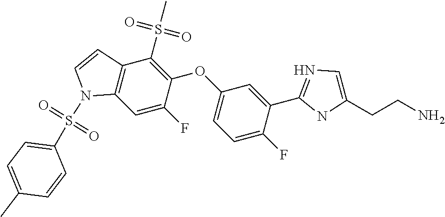 [SMILES: Cc1ccc(S(=O)(=O)n2ccc3c(S(C)(=O)=O)c(Oc4ccc(F)c(-c5ncc(CCN)[nH]5)c4)c(F)cc32)cc1]

Step E: To a stirred solution of 2-[2-[2-[2-fluoro-5-[6-fluoro-4-methylsulfonyl-1-(p-tolylsulfonyl)indol-5-yl]oxy-phenyl]-1H-imidazol-4-yl]ethyl]isoindoline-1,3-dione (1.00 eq, 2.00 g, 2.79 mmol) in ethanol (15 mL) was added hydrazine hydrate (3.00 eq, 0.42 g, 8.37 mmol) at RT. The mixture was heated at 50° C. for 3 h and concentrated. The residue was dissolved in EtOAc and washed with water. The separated organic phase was dried over anhydrous Na₂SO₄, filtered, and concentrated. The crude was purified by flash column chromatography on neutral alumina, eluting with 5-10% of methanol in DCM, to afford 2-[2-[2-fluoro-5-[6-fluoro-4-methylsulfonyl-1-(p-tolylsulfonyl)indol-5-yl]oxy-phenyl]-1H-imidazol-4-yl]ethanamine (1.00 g, 1.70 mmol, 61% yield) as an off-white solid. MS (ESI): m/z 587 [M+H]⁺.

### tert-Butyl (E)-3-[2-fluoro-3-[2-[2-fluoro-5-[6-fluoro-4-methylsulfonyl-1-(p-tolylsulfonyl)indol-5-yl]oxy-phenyl]-4,5,6,7-tetrahydro-1H-imidazo[4,5-c]pyridin-4-yl]phenyl]-2-methyl-prop-2-enoate

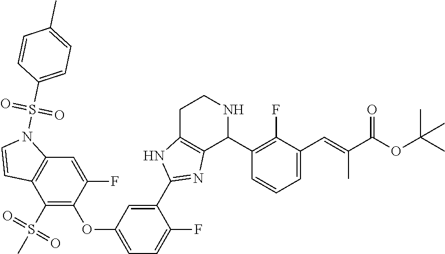 [SMILES: C/C(=C\c1cccc(C2NCCc3[nH]c(-c4cc(Oc5c(F)cc6c(ccn6S(=O)(=O)c6ccc(C)cc6)c5S(C)(=O)=O)ccc4F)nc32)c1F)C(=O)OC(C)(C)C]

Step F: To a flask containing flame dried 4 molecular sieves was added at RT a solution of 2-[2-[2-fluoro-5-[6-fluoro-4-methylsulfonyl-1-(p-tolylsulfonyl)indol-5-yl]oxy-phenyl]-1H-imidazol-4-yl]ethanamine (1.00 eq, 500 mg, 0.852 mmol) in ethanol (5 mL), followed by ethyl tert-butyl (E)-3-(2-fluoro-3-formyl-phenyl)-2-methyl-prop-2-enoate (1.20 eq, 0.27 g, 1.02 mmol) and TEA (3.00 eq, 259 mg, 2.56 mmol). The reaction mixture was stirred at 70-80° C. for 48 h. After cooling to rt, the molecular sieves were removed by filtration. The filtrate was concentrated under reduced pressure. The crude product was purified by column chromatography on silica gel, eluting with 1-2% of MeOH in DCM, to afford tert-butyl (E)-3-[2-fluoro-3-[2-[2-fluoro-5-[6-fluoro-4-methylsulfonyl-1-(p-tolylsulfonyl)indol-5-yl]oxy-phenyl]-4,5,6,7-tetrahydro-1H-imidazo[4,5-c]pyridin-4-yl]phenyl]-2-methyl-prop-2-enoate (400 mg, 0.480 mmol, 56% yield) as a pale yellow solid. MS (ESI): m/z 833 [M+H]⁺.

### tert-Butyl 3-[2-fluoro-3-[2-[2-fluoro-5-[6-fluoro-4-methylsulfonyl-1-(p-tolylsulfonyl)indol-5-yl]oxy-phenyl]-5-methyl-1,4,6,7-tetrahydroimidazo[4,5-c]pyridin-4-yl]phenyl]-2-methyl-propanoate

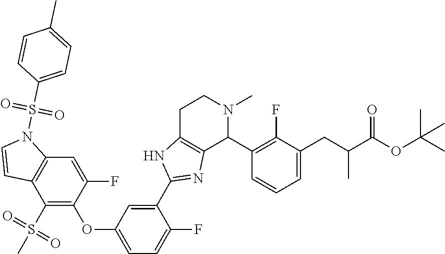 [SMILES: Cc1ccc(S(=O)(=O)n2ccc3c(S(C)(=O)=O)c(Oc4ccc(F)c(-c5nc6c([nH]5)CCN(C)C6c5cccc(CC(C)C(=O)OC(C)(C)C)c5F)c4)c(F)cc32)cc1]

Step G: To a stirred solution of tert-butyl (E)-3-[2-fluoro-3-[2-[2-fluoro-5-[6-fluoro-4-methylsulfonyl-1-(p-tolylsulfonyl)indol-5-yl]oxy-phenyl]-4,5,6,7-tetrahydro-1H-imidazo[4,5-c]pyridin-4-yl]phenyl]-2-methyl-prop-2-enoate (1.00 eq, 100 mg, 0.120 mmol) in ethanol (3 mL) was added at RT aqueous formaldehyde (37% w/v solution, 1.50 eq, 0.014 mL, 0.180 mmol) and PtO₂ (1.00 eq, 7.0 mg, 0.0308 mmol). The resulting mixture was stirred for 16 h under a hydrogen atmosphere at ambient temperature. The reaction mixture was filtered through a Celite pad, and the pad was washed with EtOH. The combined filtrates were concentrated under reduced pressure to afford tert-butyl 3-[2-fluoro-3-[2-[2-fluoro-5-[6-fluoro-4-methylsulfonyl-1-(p-tolylsulfonyl)indol-5-yl]oxy-phenyl]-5-methyl-1,4,6,7-tetrahydroimidazo[4,5-c]pyridin-4-yl]phenyl]-2-methyl-propanoate (80 mg, 0.0942 mmol, 80.00% yield) as an off-white solid. MS (ESI): m/z 849 [M+H]⁺.

### tert-Butyl 3-[2-fluoro-3-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]phenyl]-2-methyl-propanoate

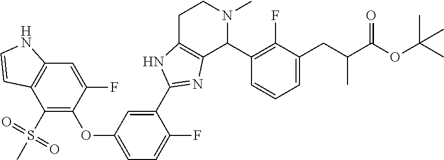 [SMILES: CC(Cc1cccc(C2c3nc(-c4cc(Oc5c(F)cc6[nH]ccc6c5S(C)(=O)=O)ccc4F)[nH]c3CCN2C)c1F)C(=O)OC(C)(C)C]

Step H: To a stirred solution of tert-butyl 3-[2-fluoro-3-[2-[2-fluoro-5-[6-fluoro-4-methylsulfonyl-1-(p-tolylsulfonyl)indol-5-yl]oxy-phenyl]-5-methyl-1,4,6,7-tetrahydroimidazo[4,5-c]pyridin-4-yl]phenyl]-2-methyl-propanoate (1.00 eq, 100 mg, 0.118 mmol) in DMF was added at RT NaH (60% dispersion in mineral oil) (10.0 eq, 32 mg, 1.39 mmol). The resulting mixture was heated at 40° C. for 1 h, quenched with water and extracted with EtOAc. The organic phase was dried over anhydrous Na₂SO₄, filtered, and concentrated under reduced pressure. The crude was purified by flash column chromatography on silica, eluting with 5-10% of MeOH in DCM, to afford tert-butyl 3-[2-fluoro-3-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]phenyl]-2-methyl-propanoate (50 mg, 0.0720 mmol, 62% yield) as an off-white solid. MS (ESI): m/z 695 [M+H]⁺.

### 3-[2-Fluoro-3-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]phenyl]-2-methyl-propanoic acid

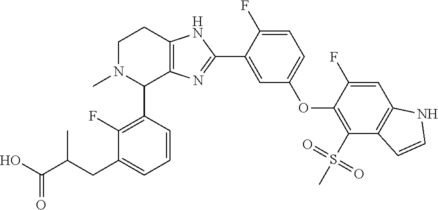 [SMILES: CC(Cc1cccc(C2c3nc(-c4cc(Oc5c(F)cc6[nH]ccc6c5S(C)(=O)=O)ccc4F)[nH]c3CCN2C)c1F)C(=O)O]

Step I: To a stirred solution of tert-butyl 3-[2-fluoro-3-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]phenyl]-2-methyl-propanoate (1.00 eq, 20 mg, 0.0288 mmol) in DCM (1 mL) was added at RT TFA (0.050 mL). The reaction solution was stirred for 3 h at RT. The volatiles were removed under reduced pressure. The crude was dissolved in a 5% MeOH in DCM mixture, basified with solid K₂C₀₃, filtered, and concentrated under reduced pressure to afford an off-white solid. The solid was triturated with MTBE, followed by pentane, and dried under reduced pressure to give 3-[2-fluoro-3-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]phenyl]-2-methyl-propanoic acid (6.0 mg, 0.00855 mmol, 30% yield) as an off-white solid. MS (ESI): m/z 639 [M+H]⁺.

### Example 8. Synthesis of 3-[2-fluoro-3-[2-[2-fluoro-5-[(6-fluoro-4-methylsulfonyl-1H-indol-5-yl)oxy]phenyl]-3-methyl-5,6,7,8-tetrahydroimidazo[1,2-a]pyrazin-8-yl]phenyl]-2-methyl-propanoic acid

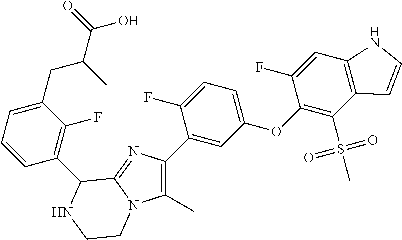 [SMILES: Cc1c(-c2cc(Oc3c(F)cc4[nH]ccc4c3S(C)(=O)=O)ccc2F)nc2n1CCNC2c1cccc(CC(C)C(=O)O)c1F]

### Ethyl (E)-3-(3-bromo-2-fluoro-phenyl)-2-methyl-prop-2-enoate

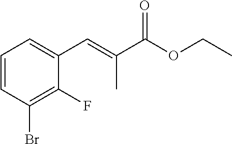 [SMILES: CCOC(=O)/C(C)=C/c1cccc(Br)c1F]

Step A: To a stirred solution of 3-bromo-2-fluoro-benzaldehyde (1.00 eq, 15.00 g, 73.9 mmol) in DCM (150 mL) was added ethyl 2-(triphenylphosphoranylidene)propionate (1.20 eq, 32.13 g, 88.7 mmol). The reaction mass was stirred at RT for 5 h. The reaction mixture was concentrated under reduced pressure. The crude product was purified by column chromatography on silica gel column chromatography, eluting with 5% EtOAc in hexane, to afford ethyl (E)-3-(3-bromo-2-fluoro-phenyl)-2-methyl-prop-2-enoate (19.00 g, 66.2 mmol, 90% yield) as a yellow syrup. MS (ESI): m/z 287, 289 [M+H]⁺.

### Ethyl (E)-3-[2-fluoro-3-(4,4,5,5-tetramethyl-1,3,2-dioxaborolan-2-yl)phenyl]-2-methyl-prop-2-enoate

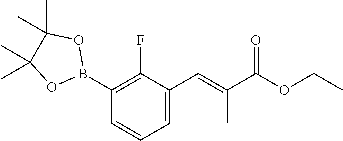 [SMILES: CCOC(=O)/C(C)=C/c1cccc(B2OC(C)(C)C(C)(C)O2)c1F]

Step B: A solution of ethyl (E)-3-(3-bromo-2-fluoro-phenyl)-2-methyl-prop-2-enoate (1.00 eq, 4.00 g, 13.9 mmol) in 1,4-dioxane (25 mL) was sparged with argon at room temperature. To this solution was added bis(pinacolato)diboron (1.50 eq, 5.31 g, 20.9 mmol), KOAc (2.00 eq, 2.73 g, 27.9 mmol) and (1,1′-bis(diphenylphosphino)ferrocene)palladium(II) dichloride (0.100 eq, 1.02 g, 1.39 mmol). The reaction mixture was heated at 110° C. for 16 h. The reaction was monitored by TLC and LC-MS. The reaction was cooled to RT and filtered through a Celite pad. The pad was washed with EtOAc (100 mL) and the combined filtrates were concentrated under reduced pressure. The crude was purified by flash column chromatography on neutral alumina, eluting with 10% EtOAc in hexanes, to give ethyl (E)-3-[2-fluoro-3-(4,4,5,5-tetramethyl-1,3,2-dioxaborolan-2-yl)phenyl]-2-methyl-prop-2-enoate (3.80 g, 11.4 mmol, 81.62% yield) MS (ESI): m/z 335 [M+H]⁺.

### Ethyl (E)-3-[2-fluoro-3-(3-methylimidazo[1,2-a]pyrazin-8-yl)phenyl]-2-methyl-prop-2-enoate

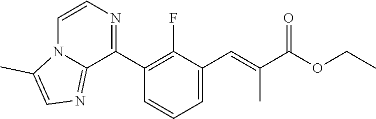 [SMILES: CCOC(=O)/C(C)=C/c1cccc(-c2nccn3c(C)cnc23)c1F]

Step C: A stirred mixture of 8-chloro-3-methyl-imidazo[1,2-a]pyrazine (1.00 eq, 1.00 g, 5.97 mmol), ethyl (E)-3-[2-fluoro-3-(4,4,5,5-tetramethyl-1,3,2-dioxaborolan-2-yl)phenyl]-2-methyl-prop-2-enoate (1.10 eq, 2.19 g, 6.56 mmol) and potassium carbonate (3.00 eq, 2.47 g, 17.9 mmol) in 1,4-dioxane (2 mL) and water (0.4 mL) was sparged with nitrogen for 5 minutes. [1,1′-bis(diphenylphosphino)ferrocene]dichloropalladium(II), complex with dichloromethane (0.1000 eq, 0.49 g, 0.597 mmol) was then added. The mixture was sparged with nitrogen and heated at 100° C. for 15 h.

After cooling to rt, the reaction mixture was diluted with EtOAc (10 mL), filtered through a Na₂SO₄ plug, and the plug was rinsed with EtOAc (3 mL). The combined filtrates were concentrated under reduced pressure. The residue was purified via flash column chromatography on silica gel, eluting with 40-45% EtOAc in hexanes, to give ethyl (E)-3-[2-fluoro-3-(3-methylimidazo[1,2-a]pyrazin-8-yl)phenyl]-2-methyl-prop-2-enoate (1.80 g, 5.30 mmol, 89% yield) MS (ESI): m/z 340 [M+H]⁺.

### Ethyl 3-[2-fluoro-3-(3-methyl-5,6,7,8-tetrahydroimidazo[1,2-a]pyrazin-8-yl)phenyl]-2-methyl-propanoate (NH F O

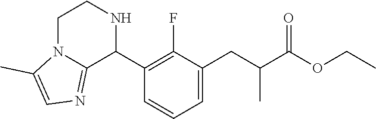 [SMILES: CCOC(=O)C(C)Cc1cccc(C2NCCn3c(C)cnc32)c1F]

Step D: To a stirred solution of ethyl (E)-3-[2-fluoro-3-(3-methylimidazo[1,2-a]pyrazin-8-yl)phenyl]-2-methyl-prop-2-enoate (1.00 eq, 1.00 g, 2.95 mmol) in ethyl acetate (10 mL) was added Pd/C (10% wt., 0.30 g). The resulting suspension was stirred at rt under 150 psi of hydrogen for 16 h. The reaction mixture was filtered through a pad of Celite, and the pad was rinsed with ethyl acetate. The combined filtrates were evaporated under reduced pressure to afford ethyl 3-[2-fluoro-3-(3-methyl-5,6,7,8-tetrahydroimidazo[1,2-a]pyrazin-8-yl)phenyl]-2-methyl-propanoate (600 mg, 1.74 mmol, 59% yield) as a pale brown syrup. MS (ESI): m/z 346 [M+H]⁺.

### tert-Butyl 8-(3-(3-ethoxy-2-methyl-3-oxopropyl)-2-fluorophenyl)-3-methyl-5,6-dihydroimidazo[1,2-a]pyrazine-7(8H)-carboxylate

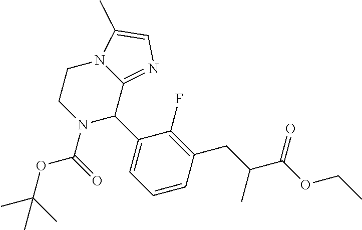 [SMILES: CCOC(=O)C(C)Cc1cccc(C2c3ncc(C)n3CCN2C(=O)OC(C)(C)C)c1F]

Step E: To a chilled (0° C.) and stirred solution of ethyl 3-[2-fluoro-3-[[5-methyl-1-[2-(methylamino)ethyl]imidazol-2-yl]methyl]phenyl]-2-methyl-propanoate (1.00 eq, 600 mg, 1.66 mmol) in DCM (15 mL) was added TEA (2.00 eq, 335 mg, 3.32 mmol) and Boc anhydride (1.50 eq, 543 mg, 2.49 mmol). The solution was stirred at RT for 16 h. The reaction mixture was diluted with DCM and Water. The separated organic phase was dried over Na₂SO₄, filtered, and concentrated. The crude product was purified by flash chromatography on silica gel, eluting with 40% ethyl acetate in hexane to give tert-butyl 8-(3-(3-ethoxy-2-methyl-3-oxopropyl)-2-fluorophenyl)-3-methyl-5,6-dihydroimidazo[1,2-a]pyrazine-7(8H)-carboxylate as a thick syrup (315 mg). MS (ESI): m/z 446 [M+H]⁺.

### tert-Butyl 8-[3-(3-ethoxy-2-methyl-3-oxo-propyl)-2-fluoro-phenyl]-2-iodo-3-methyl-6,8-dihydro-5H-imidazo[1,2-a]pyrazine-7-carboxylate

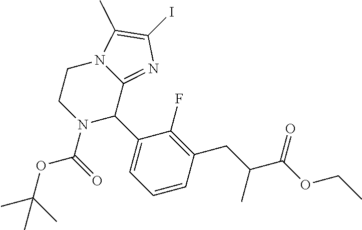 [SMILES: CCOC(=O)C(C)Cc1cccc(C2c3nc(I)c(C)n3CCN2C(=O)OC(C)(C)C)c1F]

Step F: To a stirred and chilled (0° C.) solution of ethyl 3-[3-[[1-[2-[tert-butoxycarbonyl (methyl)amino]ethyl]-5-methyl-imidazol-2-yl]methyl]-2-fluoro-phenyl]-2-methyl-propanoate (1.00 eq, 315 mg, 0.682 mmol) in 1,4-dioxane (5 mL) was added NIS (0.900 eq, 138 mg, 0.614 mmol). The solution was stirred at RT for 16 h. The reaction was quenched with saturated aqueous sodium thiosulphate solution (3 mL) and extracted with EtOAc (2×8 mL). The combined organic extracts were washed with brine (5 mL), dried over anhydrous sodium sulphate, filtered, and concentrated under reduced pressure. The crude was purified by flash column chromatography on silica gel, eluting with 5-10% EtOAc in hexanes to afford tert-butyl 8-[3-(3-ethoxy-2-methyl-3-oxo-propyl)-2-fluoro-phenyl]-2-iodo-3-methyl-6,8-dihydro-5H-imidazo[1,2-a]pyrazine-7-carboxylate (172 mg, 0.301 mmol, 44. % yield). MS (ESI): m/z 572 [M+H]⁺.

### 5-(2-Bromo-6-fluoro-3-methyl-4-nitro-phenoxy)-2-fluoro-aniline

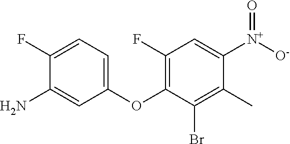 [SMILES: Cc1c([N+](=O)[O-])cc(F)c(Oc2ccc(F)c(N)c2)c1Br]

Step G: To a stirred solution of 3-bromo-1,2-difluoro-4-methyl-5-nitro-benzene (1.00 eq, 20.00 g, 79.4 mmol) in DMF (25 mL) was added at room temperature 3-amino-4-fluoro-phenol (1.00 eq, 10.09 g, 79.4 mmol), followed by potassium carbonate (2.00 eq, 21.94 g, 159 mmol). The reaction mixture was stirred at room temperature for 4 h. The reaction mixture was poured into ice-water (30 mL) and extracted with EtOAc (2×20 mL). The combined organic extracts were washed with brine (10 mL), dried over anhydrous Na₂SO₄, filtered, and concentrated under reduced pressure. The crude was purified by flash column chromatography on silica gel, eluting with 8-10% EtOAc in hexanes, to give 5-(2-bromo-6-fluoro-3-methyl-4-nitro-phenoxy)-2-fluoro-aniline (24.00 g, 66.8 mmol, 84. % yield) as a yellow solid. ¹H NMR (300 MHz, DMSO-d₆) δ 8.24 (d, 1H), 6.94 (dd, 1H), 6.09-6.04 (m, 1H), 5.33 (s, 2H), 2.5 (s, 3H)

### 2-Fluoro-5-[6-fluoro-2-[(4-methoxyphenyl)methylsulfanyl]-3-methyl-4-nitro-phenoxy]aniline

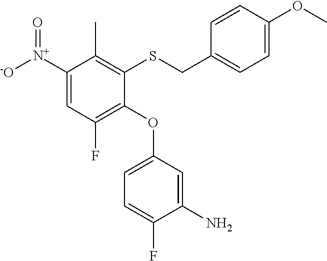

Step H: To a stirred solution of 5-(2-bromo-6-fluoro-3-methyl-4-nitro-phenoxy)-2-fluoro-aniline (1.00 eq, 11.00 g, 30.6 mmol) in 1,4-dioxane (15 mL) was added under argon 4-methoxybenzyl mercaptan (1.00 eq, 4.3 mL, 30.6 mmol), N,N-diisopropylethylamine (2.00 eq, 11 mL, 61.3 mmol), (9,9-dimethyl-9H-xanthene-4,5-diyl)bis(diphenylphosphane) (0.100 eq, 1.77 g, 3.06 mmol) and tris(dibenzylideneacetone)dipalladium(0) (0.050 eq, 1.40 g, 1.53 mmol). The reaction mixture was stirred at 100° C. for 16 h. The reaction was cooled to rt, quenched with water (20 mL) and extracted with EtOAc (2×20 mL). The combined organic extracts were washed with brine (20 mL), dried over anhydrous Na₂SO₄, filtered, and concentrated. The crude was purified by flash column chromatography on silica gel, eluting with 5-10% EtOAc in hexanes, to give 2-fluoro-5-[6-fluoro-2-[(4-methoxyphenyl)methylsulfanyl]-3-methyl-4-nitro-phenoxy]aniline (5.30 g, 12.3 mmol, 40% yield) as a light-yellow solid. ¹H NMR (300 MHz, CDCl₃) δ 7.65 (d, 1H), 7.04-7.00 (m, 2H), 6.89 (dd, 1H), 6.776.73 (m, 2H), 6.28 (dd, 1H), 6.16-6.11 (m, 1H), 3.98 (s, 2H), 3.78 (d, 2H), 3.77 (s, 3H), 2.49 (s, 3H)

### 2-(3-Amino-4-fluoro-phenoxy)-3-fluoro-6-methyl-5-nitro-benzenethiol

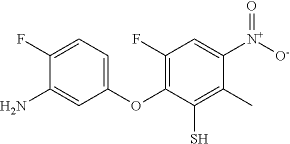 [SMILES: Cc1c([N+](=O)[O-])cc(F)c(Oc2ccc(F)c(N)c2)c1S]

Step I: To a stirred solution of 2-fluoro-5-[6-fluoro-2-[(4-methoxyphenyl)methylsulfanyl]-3-methyl-4-nitro-phenoxy]aniline (1.00 eq, 10.00 g, 23.1 mmol) in anisole (18.3 eq, 46 mL, 423 mmol) was added at RT TFA (25.9 eq, 46 mL, 599 mmol). The reaction mixture was stirred at 50° C. for 16 h. The reaction mixture was concentrated under reduced pressure and co-distilled with toluene (2×20 mL) to afford a brown colored crude product. The crude was dissolved in THE (100 mL), treated with triphenylphosphine (0.900 eq, 5.46 g, 20.8 mmol). The solution stirred for 4 h at RT. The mixture was concentrated under reduced pressure to give crude 2-(3-amino-4-fluoro-phenoxy)-3-fluoro-6-methyl-5-nitro-benzenethiol (7.20 g, 23.1 mmol, 99. % yield), which was used directly in the next step. MS (ESI): m/z 313 [M+H]⁺.

### 2-Fluoro-5-(6-fluoro-3-methyl-2-methylsulfanyl-4-nitro-phenoxy)aniline

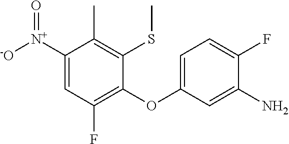

Step J: To a stirred solution of crude 2-(3-amino-4-fluoro-phenoxy)-3-fluoro-6-methyl-5-nitro-benzenethiol (1.00 eq, 7.20 g, 23.1 mmol) in acetone (70 mL) and TEA (2.00 eq, 6.4 mL, 46.1 mmol) was added slowly at rt iodomethane (1.50 eq, 2.2 mL, 34.6 mmol). The reaction mixture was stirred at RT for 2 h, quenched with water (20 mL), and extracted with EtOAc (2×20 mL). The combined organic extracts were washed with brine (20 mL), dried over anhydrous Na₂SO₄, filtered, and concentrated. The crude was purified by flash column chromatography on silica gel, eluting with 5-8% EtOAc in hexanes to afford 2-fluoro-5-(6-fluoro-3-methyl-2-methylsulfanyl-4-nitro-phenoxy)aniline (5.20 g, 15.9 mmol, 69% yield) as a yellow solid. MS (ESI): m/z 327 [M+H]⁺.

### 2-(3-Bromo-4-fluoro-phenoxy-1-fluoro-4-methyl-3-methylsulfanyl-5-nitro-benzene

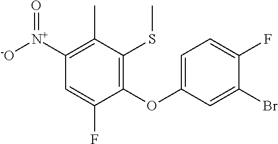

Step K: To a chilled (0° C.) and stirred solution of 2-fluoro-5-(6-fluoro-3-methyl-2-methylsulfanyl-4-nitro-phenoxy)aniline (1.00 eq, 4.00 g, 12.3 mmol) in MeCN (5 mL) was added slowly under a nitrogen atmosphere tert-butyl nitrite (1.50 eq, 2.2 mL, 18.4 mmol) followed by copper(I) bromide (1.00 eq, 1.76 g, 12.3 mmol). The reaction mixture was allowed to warm to room temperature and stirred for 4 h. The reaction was quenched with water (20 mL) and extracted with EtOAc (2×20 mL). The combined organic extracts were washed with brine (20 mL), dried over anhydrous Na₂SO₄, filtered, and concentrated. The crude was purified by flash column chromatography on silica gel, eluting with 2-6% EtOAc in hexanes, to give 2-(3-bromo-4-fluoro-phenoxy)-1-fluoro-4-methyl-3-methylsulfanyl-5-nitro-benzene (2.20 g, 5.64 mmol, 46% yield) as a colorless syrup. ¹H NMR (300 MHz, CDCl₃) δ 7.68 (d, 1H), 7.10-7.05 (m, 2H), 6.84-6.79 (m, 1H), 2.71 (s, 3H), 2.38 (s, 3H) (E)-2-(3-(3-bromo-4-fluorophenoxy)-4-fluoro-2-(methylthio)-6-nitrophenyl)-N,N-dimethylethene-1-amine

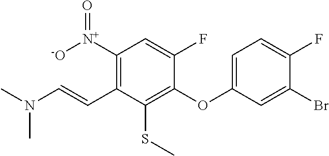

Step L: To a stirred solution of 2-(3-bromo-4-fluoro-phenoxy)-1-fluoro-4-methyl-3-methylsulfanyl-5-nitro-benzene (1.00 eq, 2.20 g, 5.64 mmol) in DMF (20 mL) was added at RT N,N-dimethylformamide dimethyl acetal (11.7 eq, 8.8 mL, 65.7 mmol). The reaction solution was stirred overnight at 100° C. The dark red solution was cooled to RT, poured into ice-water (70 mL), and extracted with ethyl acetate (30 mL×2). The combined organic extracts were washed with brine (50 mL), dried over anhydrous Na₂SO₄, filtered, and concentrated under reduced pressure to obtain the crude product as a dark brown oil (2.50 g). The crude was used in the next step without further characterization or purification.

### 5-(3-Bromo-4-fluoro-phenoxy)-6-fluoro-4-methylsulfanyl-1H-indole

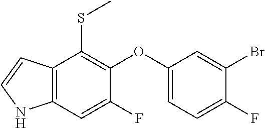 [SMILES: CSc1c(Oc2ccc(F)c(Br)c2)c(F)cc2[nH]ccc12]

Step M: To a stirred solution of crude (E)-2-[3-(3-bromo-4-fluoro-phenoxy)-4-fluoro-2-methylsulfanyl-6-nitro-phenyl]-N,N-dimethyl-ethenamine (1.00 eq, 2.50 g, 5.61 mmol) in toluene (25 mL) and acetic acid (25 mL) was added iron powder (12.0 eq, 3.76 g, 67.4 mmol). The mixture was heated at 100° C. for 6 h. The reaction was cooled to RT, diluted with water (10 mL) and EtOAc (10 mL), and stirred for 10 minutes. The mixture was filtered through a Celite pad, and the pad was washed with EtOAc (10 mL). The separated organic phase of the combined filtrates was washed with brine (10 mL), dried over anhydrous Na₂SO₄, filtered, and concentrated under reduced pressure. The residue was purified by flash chromatography on silica gel, eluting with 5-10% of EtOAc in n-hexane, to obtain 5-(3-bromo-4-fluoro-phenoxy)-6-fluoro-4-methylsulfanyl-1H-indole (1.20 g, 3.24 mmol, 57% yield) as a light-yellow syrup. MS (ESI): m/z 370, 372 [M+H]⁺.

### 2-[[5-(3-Bromo-4-fluoro-phenoxy)-6-fluoro-4-methylsulfanyl-indol-1-yl]methoxy]ethyl-trimethyl-silane

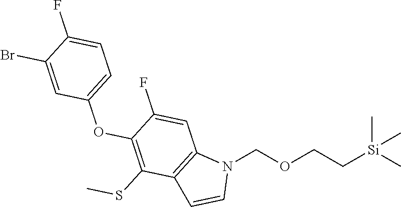 [SMILES: CSc1c(Oc2ccc(F)c(Br)c2)c(F)cc2c1ccn2COCC[Si](C)(C)C]

Step N: To a stirred solution of 5-(3-bromo-4-fluoro-phenoxy)-6-fluoro-4-methylsulfanyl-1H-indole (1.00 eq, 1.20 g, 3.24 mmol) in DMF (10 mL) was added at 0° C. sodium hydride (1.50 eq, 0.12 g, 4.86 mmol), followed by 2-(trimethylsilyl)ethoxymethyl chloride (1.10 eq, 0.65 mL, 3.57 mmol). The mixture was stirred for 2 h at RT. The reaction was quenched with ice-water (10 ml), extracted with EtOAc (10 mL×2). The combined organic extracts were washed with brine (10 mL), dried over anhydrous Na₂SO₄, filtered, and concentrated under reduced pressure. The crude was purified by column chromatography on silica gel, eluting with 4-8% EtOAc in hexanes, to obtain 2-[[5-(3-bromo-4-fluoro-phenoxy)-6-fluoro-4-methylsulfanyl-indol-1-yl]methoxy]ethyl-trimethyl-silane (1.10 g, 2.20 mmol, 68% yield) as a colorless syrup. MS (ESI): m/z 500, 502 [M+H]⁺.

### [2-Fluoro-5-[6-fluoro-4-methylsulfanyl-1-(2-trimethylsilylethoxymethyl)indol-5-yl]oxy-phenyl]boronic acid

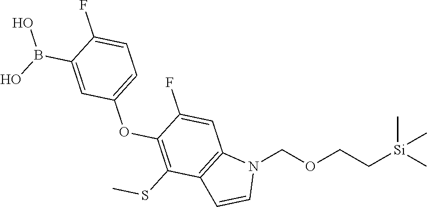 [SMILES: CSc1c(Oc2ccc(F)c(B(O)O)c2)c(F)cc2c1ccn2COCC[Si](C)(C)C]

Step O: To a chilled (0° C.) and stirred solution of 2-[[5-(3-bromo-4-fluoro-phenoxy)-6-fluoro-4-methylsulfanyl-indol-1-yl]methoxy]ethyl-trimethyl-silane (1.00 eq, 100 mg, 0.200 mmol) in THE (0.5 mL) was added under argon a solution of isopropyl magnesium chloride-lithium chloride complex (1.20 eq, 0.30 mL, 0.240 mmol). After stirring for 30 minutes at 0° C., the mixture was treated with trimethyl borate (2.60 eq, 0.058 mL, 0.520 mmol), and stirred at 0° C. for another 2 h. The reaction was quenched with saturated aqueous ammonium chloride solution (20 mL) and extracted with ethyl acetate (2×20 mL). The combined organic extracts were washed with brine (20 mL), dried over anhydrous Na₂SO₄, filtered, and concentrated under reduced pressure. The crude was purified by flash column chromatography on silica gel, eluting with 15-20% EtOAc in hexanes, to give 12-fluoro-5-16-fluoro-4-methylsulfanyl-1-(2-trimethylsilylethoxymethyl)indol-5-yl]oxy-phenyl]boronic acid (35 mg, 0.0752 mmol, 38% yield) as a colorless syrup. MS (ESI): m/z 466 [M+H]⁺.

### tert-Butyl 8-[3-(3-ethoxy-2-methyl-3-oxo-propyl)-2-fluoro-phenyl]-2-[2-fluoro-5-[6-fluoro-4-methylsulfanyl-]-(2-trimethylsilylethoxymethyl)indol-5-yl]oxy-phenyl]-3-methyl-6,8-dihydro-5H-imidazo[1,2-a]pyrazine-7-carboxylate

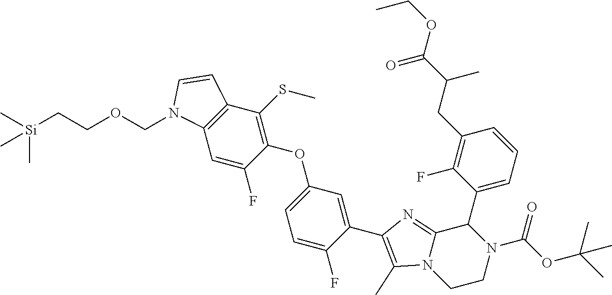 [SMILES: CCOC(=O)C(C)Cc1cccc(C2c3nc(-c4cc(Oc5c(F)cc6c(ccn6COCC[Si](C)(C)C)c5SC)ccc4F)c(C)n3CCN2C(=O)OC(C)(C)C)c1F]

Step P: To a stirred solution of tert-butyl 8-[3-(3-ethoxy-2-methyl-3-oxo-propyl)-2-fluoro-phenyl]-2-iodo-3-methyl-6,8-dihydro-5H-imidazo[1,2-a]pyrazine-7-carboxylate (1.00 eq, 70 mg, 0.123 mmol) and [2-fluoro-5-[6-fluoro-4-methylsulfanyl-1-(2-trimethylsilylethoxymethyl)indol-5-yl]oxy-phenyl]boronic acid (1.20 eq, 68 mg, 0.147 mmol) in 1,4-dioxane (1.5 mL) and water (0.5 mL) was added under argon potassium carbonate (3.00 eq, 51 mg, 0.368 mmol) and [1,1′-bis(diphenylphosphino)ferrocene]dichloropalladium(II), complex with dichloromethane (0.05 eq, 5.0 mg, 0.00613 mmol). The mixture was stirred at 80° C. for 2 h, cooled to rt, quenched with water (10 mL), and extracted with EtOAc (2×10 mL). The combined organic extracts were washed with brine (10 mL), dried over anhydrous sodium sulphate, filtered, and concentrated. The crude was purified by flash column chromatography on silica gel, eluting with 15-18% EtOAc in hexanes, to give tert-butyl 8-[3-(3-ethoxy-2-methyl-3-oxo-propyl)-2-fluoro-phenyl]-2-[2-fluoro-5-[6-fluoro-4-methylsulfanyl-1-(2-trimethylsilylethoxymethyl)indol-5-yl]oxy-phenyl]-3-methyl-6,8-dihydro-5H-imidazo[1,2-a]pyrazine-7-carboxylate (70 mg, 0.0809 mmol, 66% yield) as an off-white solid. MS (ESI): m/z 865 [M+H]⁺.

### tert-Butyl 8-[3-(3-ethoxy-2-methyl-3-oxo-propyl)-2-fluoro-phenyl]-2-[2-fluoro-5-[6-fluoro-4-methylsulfonyl-]-(2-trimethylsilylethoxymethyl)indol-5-yl]oxy-phenyl]-3-methyl-6,8-dihydro-5H-imidazo[1,2-a]pyrazine-7-carboxylate

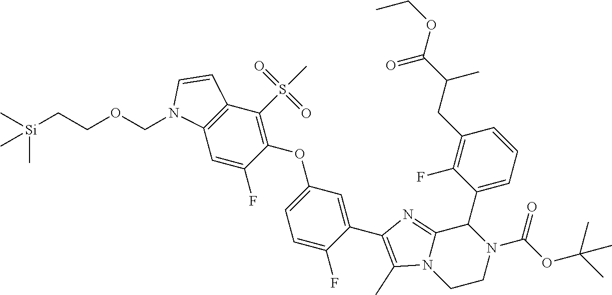 [SMILES: CCOC(=O)C(C)Cc1cccc(C2c3nc(-c4cc(Oc5c(F)cc6c(ccn6COCC[Si](C)(C)C)c5S(C)(=O)=O)ccc4F)c(C)n3CCN2C(=O)OC(C)(C)C)c1F]

Step Q: To a stirred solution of tert-butyl 8-[3-(3-ethoxy-2-methyl-3-oxo-propyl)-2-fluoro-phenyl]-2-[2-fluoro-5-[6-fluoro-4-methylsulfanyl-1-(2-trimethylsilylethoxymethyl)indol-5-yl]oxy-phenyl]-3-methyl-6,8-dihydro-5H-imidazo[1,2-a]pyrazine-7-carboxylate (1.00 eq, 450 mg, 0.520 mmol) in 2-methyltetrahydrofuran (15 mL) was added slowly at RT a solution of oxone (5.00 eq, 1599 mg, 2.60 mmol) in water (15 mL). The reaction mixture was stirred at RT for 24 h, quenched with ice-cold water, and extracted with EtOAc (2×20 mL). The combined organic extracts were washed with brine (20 mL), dried over anhydrous Na₂SO₄, filtered, and concentrated. The crude was purified by flash column chromatography on silica gel, eluting with 25-30% EtOAc in hexanes, to afford tert-butyl 8-[3-(3-ethoxy-2-methyl-3-oxo-propyl)-2-fluoro-phenyl]-2-[2-fluoro-5-[6-fluoro-4-methylsulfonyl-1-(2-trimethylsilylethoxymethyl)indol-5-yl]oxy-phenyl]-3-methyl-6,8-dihydro-5H-imidazo[1,2-a]pyrazine-7-carboxylate (250 mg, 0.279 mmol, 54% yield) as an off-white solid. MS (ESI): m/z 897 [M+H]⁺.

### tert-Butyl 8-(3-(3-ethoxy-2-methyl-3-oxopropyl)-2-fluorophenyl)-2-(2-fluoro-5-((6-fluoro-4-(methylsulfonyl)-1H-indol-5-yl)oxy)phenyl)-3-methyl-5,6-dihydroimidazo[1,2-a]pyrazine-7(8H)-carboxylate

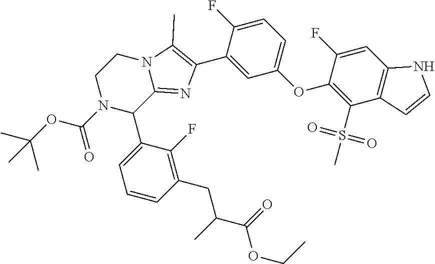 [SMILES: CCOC(=O)C(C)Cc1cccc(C2c3nc(-c4cc(Oc5c(F)cc6[nH]ccc6c5S(C)(=O)=O)ccc4F)c(C)n3CCN2C(=O)OC(C)(C)C)c1F]

Step R: To a stirred solution of tert-butyl 8-[3-(3-ethoxy-2-methyl-3-oxo-propyl)-2-fluoro-phenyl]-2-[2-fluoro-5-[6-fluoro-4-methylsulfonyl-1-(2-trimethylsilylethoxymethyl)indol-5-yl]oxy-phenyl]-3-methyl-6,8-dihydro-5H-imidazo[1,2-a]pyrazine-7-carboxylate (1.00 eq, 100 mg, 0.111 mmol) in THE (3 mL) was added at RT TBAF (1.0 M in THF, 10 eq, 1.2 mL). The reaction mixture was stirred at 80° C. for 4 h and concentrated. The residue was subjected to an aqueous workup to obtain a crude product (180 mg) that was carried directly to the next step. MS (ESI): m/z 767 [M+H]⁺ observed.

### 3-(3-(7-(tert-Butoxycarbonyl)-2-(2-fluoro-5-((6-fluoro-4-(methylsulfonyl)-1H-indol-5-yl)oxy)phenyl)-3-methyl-5,6,7,8-tetrahydroimidazo[1,2-a]pyrazin-8-yl)-2-fluorophenyl)-2-methylpropanoic acid

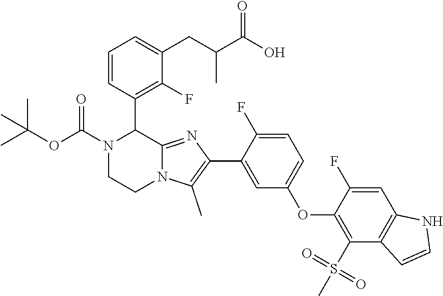 [SMILES: Cc1c(-c2cc(Oc3c(F)cc4[nH]ccc4c3S(C)(=O)=O)ccc2F)nc2n1CCN(C(=O)OC(C)(C)C)C2c1cccc(CC(C)C(=O)O)c1F]

Step S: To a cooled (10-15° C.) and stirred solution of crude tert-butyl 8-[3-(3-ethoxy-2-methyl-3-oxo-propyl)-2-fluoro-phenyl]-2-[2-fluoro-5-[(6-fluoro-4-methylsulfonyl-1H-indol-5-yl)oxy]phenyl]-3-methyl-6,8-dihydro-5H-imidazo[1,2-a]pyrazine-7-carboxylate (180 mg, 0.235 mmol) in methanol (2 mL) was added a solution of NaOH (5.00 eq, 47 mg, 1.17 mmol) in water (2 mL). The reaction was allowed to warm to room temperature and stirred for 5 h. The reaction mixture was diluted with ice-cold water (20 mL), and acidified with 1N aqueous HCl. The solid formed were isolated by filtration, washed with water (10 mL), and dried. The crude was purified by flash column chromatography on silica gel, eluting with 5-10% MeOH in DCM to afford pure 3-(3-(7-(tert-butoxycarbonyl)-2-(2-fluoro-5-((6-fluoro-4-(methylsulfonyl)-1H-indol-5-yl)oxy)phenyl)-3-methyl-5,6,7,8-tetrahydroimidazo[1,2-a]pyrazin-8-yl)-2-fluorophenyl)-2-methylpropanoic acid (25 mg), as well as some less pure desired product (80 mg). MS (ESI): m/z 739 [M+H]⁺.

### 3-[2-Fluoro-3-[2-[2-fluoro-5-[(6-fluoro-4-methylsulfonyl-1H-indol-5-yl)oxy]phenyl]-3-methyl-5,6,7,8-tetrahydroimidazo[1,2-a]pyrazin-8-yl]phenyl]-2-methyl-propanoic acid

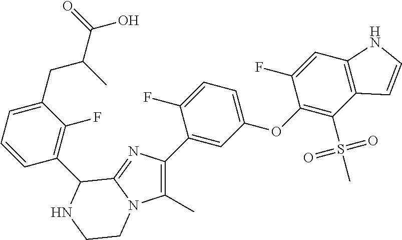 [SMILES: Cc1c(-c2cc(Oc3c(F)cc4[nH]ccc4c3S(C)(=O)=O)ccc2F)nc2n1CCNC2c1cccc(CC(C)C(=O)O)c1F]

Step T: To a cooled (5-10° C.) and stirred solution of 3-[3-[7-tert-butoxycarbonyl-2-[2-fluoro-5-[(6-fluoro-4-methylsulfonyl-1H-indol-5-yl)oxy]phenyl]-3-methyl-6,8-dihydro-5H-imidazo[1,2-a]pyrazin-8-yl]-2-fluoro-phenyl]-2-methyl-propanoic acid (1.00 eq, 40 mg, 0.0541 mmol) in DCM (1.5 mL) was added TFA (10.0 eq, 0.042 mL, 0.541 mmol). The reaction was allowed to warm up to room temperature and stirred overnight. The reaction mixture was concentrated under reduced pressure. The residue was suspended in 5 mL of water, basified with saturated aqueous NaHCO₃ solution, and extracted with EtOAc (3×10 mL). The combined organic extracts were dried over sodium sulphate, filtered, and concentrated. The crude was purified by preparative TLC (silica gel), developing with 10% MeOH in DCM to give 3-[2-fluoro-3-[2-[2-fluoro-5-[(6-fluoro-4-methylsulfonyl-1H-indol-5-yl)oxy]phenyl]-3-methyl-5,6,7,8-tetrahydroimidazo[1,2-a]pyrazin-8-yl]phenyl]-2-methyl-propanoic acid (12 mg, 0.0173 mmol, 32% yield) as an off-white solid. MS (ESI): m/z 639 [M+H]⁺.

### Example 9. Synthesis of 3-[3-[2-[5-[(4,6-difluoro-1H-indol-5-yl)oxy]-2-fluoro-phenyl]-3-methyl-5,6,7,8-tetrahydroimidazo[1,2-a]pyrazin-8-yl]-2-fluoro-phenyl]propane-1,2-diol

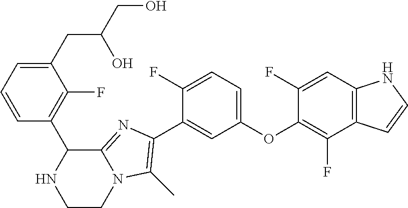 [SMILES: Cc1c(-c2cc(Oc3c(F)cc4[nH]ccc4c3F)ccc2F)nc2n1CCNC2c1cccc(CC(O)CO)c1F]

### [5-[(4,6-Difluoro-1H-indol-5-yl)oxy]-2-fluoro-phenyl]boronic acid

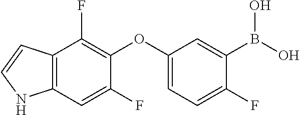 [SMILES: OB(O)c1cc(Oc2c(F)cc3[nH]ccc3c2F)ccc1F]

Step A: To a stirred solution of 5-(3-bromo-4-fluoro-phenoxy)-4,6-difluoro-1H-indole (1.00 eq, 1.00 g, 2.92 mmol) in dry THE (3 mL) was added at 0° C. under argon a solution of isopropyl magnesium chloride lithium chloride complex (1.3 M, 2.50 eq, 1.06 g, 7.31 mmol). The solution was stirred for 1 h, treated with trimethylborate (2.00 eq, 0.61 g, 5.85 mmol), and stirred at 0° C. for another 2 h. The reaction was diluted with ethyl acetate and washed with 0.1N aqueous HCl (40 mL). The organic phase was dried over Na₂SO₄, filtered, and concentrated under reduced pressure. The crude was triturated with n-pentane to afford [5-[(4,6-difluoro-1H-indol-5-yl)oxy]-2-fluoro-phenyl]boronic acid (0.85 g, 2.77 mmol, 95% yield) as a pale-yellow solid. MS (ESI): m/z 306 [M−H]⁻.

### 8-(3-Bromo-2-fluoro-phenyl)-3-methyl-imidazo[1,2-a]pyrazine

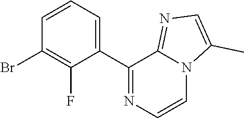 [SMILES: Cc1cnc2c(-c3cccc(Br)c3F)nccn12]

Step B: To a stirred solution of 8-chloro-3-methyl-imidazo[1,2-a]pyrazine (1.00 eq, 2.00 g, 11.9 mmol) in 1,4-dioxane (24 mL) and water (6 mL) were added under an argon atmosphere (3-bromo-2-fluoro-phenyl)boronic acid (1.00 eq, 2.61 g, 11.9 mmol), potassium carbonate (3.00 eq, 4.95 g, 35.8 mmol) and [1,1′-bis(diphenylphosphino)ferrocene]dichloropalladium(II), complex with dichloromethane (0.0500 eq, 0.49 g, 0.597 mmol). The resulting mixture was stirred at 80° C. for 12 h. The reaction mixture was diluted with ethyl acetate and filtered through Celite. The organic phase was isolated and concentrated under reduced pressure. The residue was purified by flash column chromatography on silica gel to afford 8-(3-bromo-2-fluoro-phenyl)-3-methyl-imidazo[1,2-a]pyrazine (2.30 g, 7.51 mmol, 63% yield) as a yellow solid. MS (ESI): m/z 308, 306 [M+H]⁺.

### 8-(3-Allyl-2-fluoro-phenyl)-3-methyl-imidazo[1,2-a]pyrazine

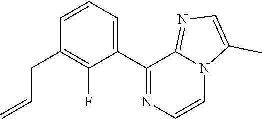 [SMILES: C=CCc1cccc(-c2nccn3c(C)cnc23)c1F]

Step C: To a stirred solution of 8-(3-bromo-2-fluoro-phenyl)-3-methyl-imidazo[1,2-a]pyrazine (1.00 eq, 2.00 g, 6.53 mmol) in 1,4-dioxane (20 mL) and water (5 mL) were added under an argon atmosphere 2-allyl-4,4,5,5-tetramethyl-1,3,2-dioxaborolane (5.00 eq, 5.49 g, 32.7 mmol), potassium carbonate (3.00 eq, 2.71 g, 19.6 mmol) and [1,1′-Bis(diphenylphosphino)ferrocene|dichloropalladium(1I), complex with dichloromethane (0.100 eq, 0.53 g, 0.653 mmol). The mixture was stirred for 12 h at 80° C., cooled to rt and diluted with ethyl acetate. The organic phase was isolated and concentrated under reduced pressure. The residue was purified by flash column chromatography on silica gel to afford 8-(3-allyl-2-fluoro-phenyl)-3-methyl-imidazo[1,2-a]pyrazine (1.60 g, 5.99 mmol, 92% yield) as a yellow solid. MS (ESI): m/z 268 [M+H]⁺.

### 3-[2-Fluoro-3-(3-methylimidazo[1, 2-a]pyrazin-8-yl)phenylpropane-1,2-diol

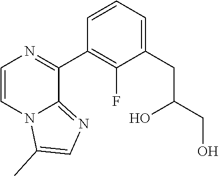 [SMILES: Cc1cnc2c(-c3cccc(CC(O)CO)c3F)nccn12]

Step D: To a stirred and chilled (0° C.) mixture of potassium hexacyanoferrate(III) (3.00 eq, 7.39 g, 22.4 mmol), potassium carbonate (3.00 eq, 3.10 g, 22.4 mmol), hydroquinidine 1,4-phthalazinediyl diether (0.0150 eq, 87 mg, 0.112 mmol), potassium hexachloroosmate (IV) (0.00107 eq, 3.8 mg, 0.00800 mmol), methanesulfonamide (1.00 eq, 712 mg, 7.48 mmol), tert-butanol (60 mL) and water (60 mL) was added a solution of 8-(3-allyl-2-fluoro-phenyl)-3-methyl-imidazo[1,2-a]pyrazine (1.00 eq, 2.00 g, 7.48 mmol) in tert-butanol (30 mL). The reaction was allowed to warm up to rt and stirred for 36 h, quenched by adding solid sodium sulfite, and extracted with ethyl acetate (2×100 mL). The combined organic extracts were washed with brine (40 mL), dried over sodium sulfate, filtered, and concentrated. The crude was purified by flash column chromatography on silica gel to give 3-[2-fluoro-3-(3-methylimidazo[1,2-a]pyrazin-8-yl)phenyl]propane-1,2-diol (1.70 g, 5.64 mmol, 75% yield) as a yellow solid. MS (ESI): m/z 302 [M+H]⁺.

### 8-(3-((2,2-Dimethyl-1,3-dioxolan-4-yl)methyl)-2-fluorophenyl)-3-methylimidazo[1,2-a]pyrazine

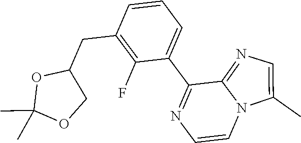

Step E: To a stirred solution of 3-[2-fluoro-3-(3-methylimidazo[1,2-a]pyrazin-8-yl)phenyl]propane-1,2-diol (1.00 eq, 1.70 g, 5.64 mmol) in acetone (10 mL) was added 2,2-dimethoxypropane (4.0 mL). The mixture was stirred at RT for 16 h. The solvent was removed under reduced pressure. The residue was purified by flash column chromatography on silica gel to afford 8-(3-((2,2-dimethyl-1,3-dioxolan-4-yl)methyl)-2-fluorophenyl)-3-methylimidazo[1,2-a]pyrazine (600 mg, 1.76 mmol, 31% yield). MS (ESI): m/z 342 [M+H]⁺.

### 8-(3-((2,2-Dimethyl-1,3-dioxolan-4-yl)methyl)-2-fluorophenyl)-3-methyl-5,6,7,8-tetrahydroimidazo[1,2-a]pyrazine

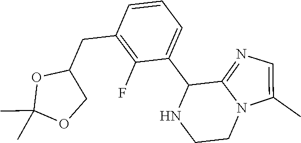

Step F: To a stirred solution of 8-[3-[(2,2-dimethyl-1,3-dioxolan-4-yl)methyl]-2-fluoro-phenyl]-3-methyl-imidazo[1,2-a]pyrazine (1.00 eq, 600 mg, 1.76 mmol) in ethanol (6 mL) was added platinum(IV) oxide (0.100 eq, 40 mg, 0.176 mmol). The mixture stirred at ambient temperature and pressure under hydrogen for 16 h. The solids were removed by filtration through a Celite plug. The filtrate was concentrated under reduced pressure. The crude product 8-(3-((2,2-dimethyl-1,3-dioxolan-4-yl)methyl)-2-fluorophenyl)-3-methyl-5,6,7,8-tetrahydroimidazo[1,2-a]pyrazine was taken to the next step without purification. MS (ESI): m/z 346 [M+H]⁺.

### tert-Butyl 8-[3-[(2,2-dimethyl-1,3-dioxolan-4-yl)methyl]-2-fluoro-phenyl]-3-methyl-6,8-dihydro-5H-imidazo[1,2-a]pyrazine-7-carboxylate

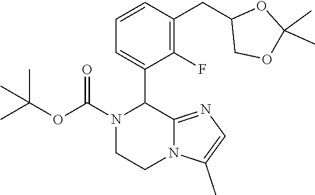 [SMILES: Cc1cnc2n1CCN(C(=O)OC(C)(C)C)C2c1cccc(CC2COC(C)(C)O2)c1F]

Step G: To a chilled (0° C.) and stirred solution of 8-[3-[(2,2-dimethyl-1,3-dioxolan-4-yl)methyl]-2-fluoro-phenyl]-3-methyl-5,6,7,8-tetrahydroimidazo[1,2-a]pyrazine (1.00 eq, 607 mg, 1.76 mmol) in DCM (8 mL) was added Boc anhydride (1.30 eq, 0.53 mL, 2.28 mmol) and TEA (3.00 eq, 0.73 mL, 5.27 mmol). The reaction was stirred at RT for 4 h, diluted with DCM, washed with saturated aqueous NaHCO₃ and brine. The organic phase was concentrated under reduced pressure. The residue was purified by flash column chromatography on silica gel to afford tert-butyl 8-[3-[(2,2-dimethyl-1,3-dioxolan-4-yl)methyl]-2-fluoro-phenyl]-3-methyl-6,8-dihydro-5H-imidazo[1,2-a]pyrazine-7-carboxylate (600 mg, 1.35 mmol, 77% yield) as a brown syrup. MS (ESI): m/z 446 [M+H]⁺.

### tert-Butyl 8-[3-[(2,2-dimethyl-1,3-dioxolan-4-yl)methyl]-2-fluoro-phenyl]-2-iodo-3-methyl-6,8-dihydro-5H-imidazo[1, 2-a]pyrazine-7-carboxylate

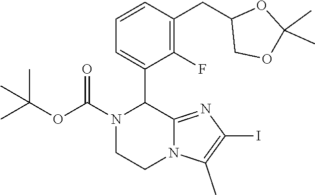 [SMILES: Cc1c(I)nc2n1CCN(C(=O)OC(C)(C)C)C2c1cccc(CC2COC(C)(C)O2)c1F]

Step H: To a chilled (0° C.) and stirred solution of tert-butyl 8-[3-[(2,2-dimethyl-1,3-dioxolan-4-yl)methyl]-2-fluoro-phenyl]-3-methyl-6,8-dihydro-5H-imidazo[1,2-a]pyrazine-7-carboxylate (1.00 eq, 600 mg, 1.35 mmol) in acetonitrile (6 mL) was added portion wise NIS (1.00 eq, 303 mg, 1.35 mmol). The reaction mixture was slowly warmed to RT, and stirred for 48 h. The reaction mixture was washed with saturated aqueous sodium thiosulfate, and the organic phase was concentrated under reduced pressure. The residue was purified by flash column chromatography on silica to afford tert-butyl 8-[3-[(2,2-dimethyl-1,3-dioxolan-4-yl)methyl]-2-fluoro-phenyl]-2-iodo-3-methyl-6,8-dihydro-5H-imidazo[1,2-a]pyrazine-7-carboxylate (600 mg, 1.05 mmol, 78% yield) as a white foam. MS (ESI): m/z 572 [M+H]⁺.

### tert-Butyl 2-[5-[(4,6-difluoro-1H-indol-5-yl)oxy]-2-fluoro-phenyl]-8-[3-[(2,2-dimethyl-1,3-dioxolan-4-yl)methyl]-2-fluoro-phenyl]-3-methyl-6,8-dihydro-5H-imidazo[1,2-a]pyrazine-7-carboxylate

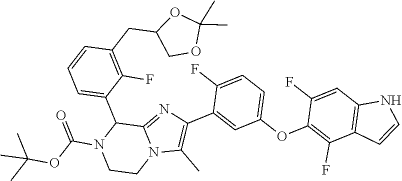 [SMILES: Cc1c(-c2cc(Oc3c(F)cc4[nH]ccc4c3F)ccc2F)nc2n1CCN(C(=O)OC(C)(C)C)C2c1cccc(CC2COC(C)(C)O2)c1F]

Step I: To a stirred solution of tert-butyl 8-[3-[(2,2-dimethyl-1,3-dioxolan-4-yl)methyl]-2-fluoro-phenyl]-2-iodo-3-methyl-6,8-dihydro-5H-imidazo[1,2-a]pyrazine-7-carboxylate (1.00 eq, 100 mg, 0.175 mmol) in 1,4-dioxane (2 mL) and water (0.5 mL) were added under an argon atmosphere [5-[(4,6-difluoro-1H-indol-5-yl)oxy]-2-fluoro-phenyl]boronic acid (1.10 eq, 59 mg, 0.193 mmol), potassium carbonate (1.00 eq, 24 mg, 0.175 mmol), and [1,1′-bis(diphenylphosphino)ferrocene]dichloropalladium(II), complex with dichloromethane (0.100 eq, 14 mg, 0.0175 mmol). The reaction mixture was stirred for 12 h at 80° C., cooled to rt, diluted with ethyl acetate, and filtered through Celite. The filtrate was concentrated under reduced pressure. The residue was purified by flash column chromatography on silica to afford tert-butyl 2-[5-[(4,6-difluoro-1H-indol-5-yl)oxy]-2-fluoro-phenyl]-8-[3-[(2,2-dimethyl-1,3-dioxolan-4-yl)methyl]-2-fluoro-phenyl]-3-methyl-6,8-dihydro-5H-imidazo[1,2-a]pyrazine-7-carboxylate (90 mg, 0.127 mmol, 73% yield) as a white solid. MS (ESI): m/z 707 [M+H]⁺.

### 3-[3-[2-[5-[(4,6-Difluoro-1H-indol-5-yl)oxy]-2-fluoro-phenyl]-3-methyl-5,6,7,8-tetrahydroimidazo[1,2-a]pyrazin-8-yl]-2-fluoro-phenyl]propane-1,2-diol

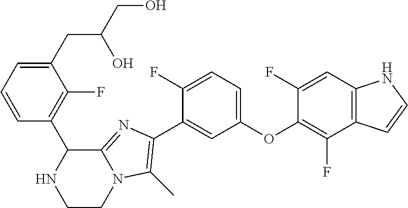 [SMILES: Cc1c(-c2cc(Oc3c(F)cc4[nH]ccc4c3F)ccc2F)nc2n1CCNC2c1cccc(CC(O)CO)c1F]

Step J: A chilled (0° C.) and stirred solution of tert-butyl 2-[5-[(4,6-difluoro-1H-indol-5-yl)oxy]-2-fluoro-phenyl]-8-[3-[(2,2-dimethyl-1,3-dioxolan-4-yl)methyl]-2-fluoro-phenyl]-3-methyl-6,8-dihydro-5H-imidazo[1,2-a]pyrazine-7-carboxylate (1.00 eq, 70 mg, 0.0990 mmol) in DCM (2 mL) was treated with DCM acidified with hydrochloric acid. The reaction mixture was stirred at 0° C. for 1 h. The solvent was evaporated under reduced pressure. The residue was purified by prep-HPLC to obtain 3-[3-[2-[5-[(4,6-difluoro-1H-indol-5-yl)oxy]-2-fluoro-phenyl]-3-methyl-5,6,7,8-tetrahydroimidazo[1,2-a]pyrazin-8-yl]-2-fluoro-phenyl]propane-1,2-diol (3.5 mg, 0.00577 mmol, 6% yield) as an off-white solid. MS (ESI): m/z 567 [M+H]⁺.

### Example 10. Synthesis of ethyl 3-[3-[2-[5-[(4,6-difluoro-1H-indol-5-yl)oxy]-2-fluoro-phenyl]-3-methyl-5,6,7,8-tetrahydroimidazo[1,2-a]pyrazin-8-yl]-2-fluoro-phenyl]-2-methyl-propanoate

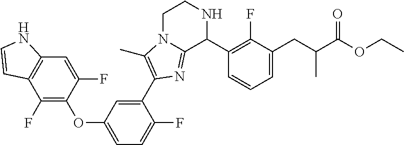 [SMILES: CCOC(=O)C(C)Cc1cccc(C2NCCn3c2nc(-c2cc(Oc4c(F)cc5[nH]ccc5c4F)ccc2F)c3C)c1F]

### tert-Butyl 2-[5-[(4,6-difluoro-1H-indol-5-yl)oxy]-2-fluoro-phenyl]-8-[3-(3-ethoxy-2-methyl-3-oxo-propyl)-2-fluoro-phenyl]-3-methyl-6,8-dihydro-5H-imidazo[1,2-a]pyrazine-7-carboxylate

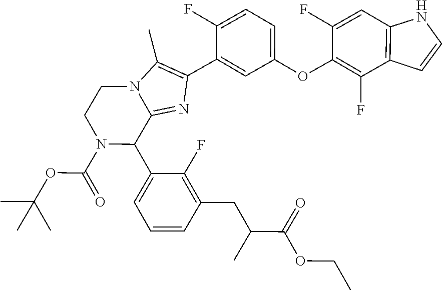 [SMILES: CCOC(=O)C(C)Cc1cccc(C2c3nc(-c4cc(Oc5c(F)cc6[nH]ccc6c5F)ccc4F)c(C)n3CCN2C(=O)OC(C)(C)C)c1F]

Step A: A stirred mixture of tert-butyl 8-[3-(3-ethoxy-2-methyl-3-oxo-propyl)-2-fluoro-phenyl]-2-iodo-3-methyl-6,8-dihydro-5H-imidazo[1,2-a]pyrazine-7-carboxylate (Step F; Example 8) (1.00 eq, 372 mg, 0.651 mmol), [5-[(4,6-difluoro-1H-indol-5-yl)oxy]-2-fluoro-phenyl]boronic acid (1.00 eq, 200 mg, 0.651 mmol), potassium carbonate (3.00 eq, 270 mg, 1.95 mmol), 1,4-dioxane (6 mL) and water (0.8 mL) was sparged with nitrogen for 5 min. 1,1′-bis(diphenylphosphino)ferrocene]dichloropalladium(II), complex with dichloromethane (0.100 eq, 53 mg, 0.0651 mmol) was added next, sparged briefly with nitrogen and heated at 80° C. Reaction progress was monitored by TLC and LC-MS. Upon depletion of the starting material iodide, the reaction was diluted with ethyl acetate, dried over anhydrous Na₂SO₄, filtered, and concentrated under reduced pressure. The crude was purified by flash column chromatography on silica gel, eluting with 20% EtOAc in hexanes to afford tert-butyl 2-[5-[(4,6-difluoro-1H-indol-5-yl)oxy]-2-fluoro-phenyl]-8-[3-(3-ethoxy-2-methyl-3-oxo-propyl)-2-fluoro-phenyl]-3-methyl-6,8-dihydro-5H-imidazo[1,2-a]pyrazine-7-carboxylate (250 mg, 0.354 mmol, 54% yield). MS (ESI): m/z 707 [M+H]⁺.

### Ethyl 3-[3-[2-[5-[(4,6-difluoro-1H-indol-5-yl)oxy]-2-fluoro-phenyl]-3-methyl-5,6,7,8-tetrahydroimidazo[1,2-a]pyrazin-8-yl]-2-fluoro-phenyl]-2-methyl-propanoate

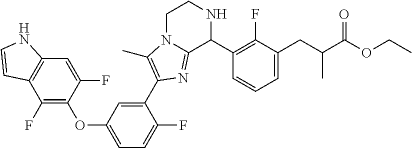 [SMILES: CCOC(=O)C(C)Cc1cccc(C2NCCn3c2nc(-c2cc(Oc4c(F)cc5[nH]ccc5c4F)ccc2F)c3C)c1F]

Step B: To a stirred and chilled (0° C.) solution of tert-butyl 2-[5-[(4,6-difluoro-1H-indol-5-yl)oxy]-2-fluoro-phenyl]-8-[3-(3-ethoxy-2-methyl-3-oxo-propyl)-2-fluoro-phenyl]-3-methyl-6,8-dihydro-5H-imidazo[1,2-a]pyrazine-7-carboxylate (1.00 eq, 190 mg, 0.269 mmol) in DCM was added under a N2 atmosphere TFA (5.00 eq, 153 mg, 1.35 mmol), and stirring continued in cold bath for 6 h. The reaction was monitored by TLC and LC-MS. After completion, the reaction was concentrated under reduced pressure. The crude was purified by flash chromatography on silica gel, eluting with 70% EtOAc in hexane, to afford ethyl 3-[3-[2-[5-[(4,6-difluoro-1H-indol-5-yl)oxy]-2-fluoro-phenyl]-3-methyl-5,6,7,8-tetrahydroimidazo[1,2-a]pyrazin-8-yl]-2-fluoro-phenyl]-2-methyl-propanoate (90 mg, 0.141 mmol, 52% yield). MS (ESI): m/z 607 [M+H]⁺.

### Example 11. Synthesis of 3-[3-[2-[5-[(4,6-difluoro-1H-indol-5-yl)oxy]-2-fluoro-phenyl]-5-methyl-1,4,6,7-tetrahydroimidazo[4,5-c]pyridin-4-yl]-2-fluoro-phenyl]-2-methyl-propanoic acid

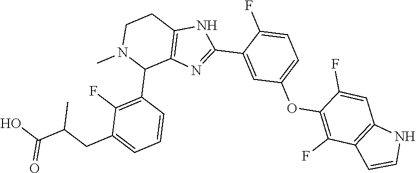 [SMILES: CC(Cc1cccc(C2c3nc(-c4cc(Oc5c(F)cc6[nH]ccc6c5F)ccc4F)[nH]c3CCN2C)c1F)C(=O)O]

### 5-[4,6-Difluoro-1-(2-trimethylsilylethoxymethyl)indol-5-yl]oxy-2-fluoro-benzonitrile

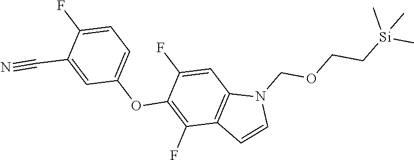 [SMILES: C[Si](C)(C)CCOCn1ccc2c(F)c(Oc3ccc(F)c(C#N)c3)c(F)cc21]

Step A: To a suspension of sodium hydride (1.50 eq, 1.78 g, 37.2 mmol) in DMF (50 mL) was added a solution 5-[(4,6-difluoro-1H-indol-5-yl)oxy]-2-fluoro-benzonitrile (1.00 eq, 7.14 g, 24.8 mmol) in DMF (20 mL) at 0° C. The reaction mixture was stirred at 0° C. for 30 minutes and 2-(trimethylsilyl)ethoxymethyl chloride (1.10 eq, 4.8 mL, 27.3 mmol) was added dropwise at 0° C. The mixture was warmed to room temperature and stirred for 3 h. The reaction mixture was poured in 200 mL ice-cold water and extracted with ethyl acetate (100 mL). The organic phase was washed with water (2×50 mL) and brine (50 mL). The organic phase was isolated, dried over anhydrous Na₂SO₄, filtered, and concentrated under reduced pressure. The crude was purified by column chromatography on silica gel, eluting with 0-7% EtOAc in hexanes to obtain 5-[4,6-difluoro-1-(2-trimethylsilylethoxymethyl)indol-5-yl]oxy-2-fluoro-benzonitrile (7.50 g, 17.0 mmol, 68% yield) as a light-yellow syrup. MS (ESI): m/z 419 [M+H]⁺.

### 5-((4,6-Difluoro-1-((2-(trimethylsilyl)ethyoxy)menthyl)-1H-indol-5-yl)oxy)-2-fluorobenzimidamide

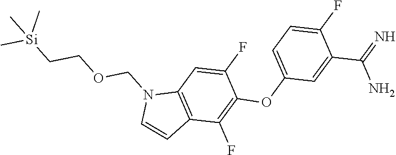 [SMILES: C[Si](C)(C)CCOCn1ccc2c(F)c(Oc3ccc(F)c(C(=N)N)c3)c(F)cc21]

Step B: To a stirred solution of 5-[4,6-difluoro-1-(2-trimethylsilylethoxymethyl)indol-5-yl]oxy-2-fluoro-benzonitrile (1.00 eq, 7.50 g, 17.9 mmol) in dry THF (70 mL) was added at 0° C. LiHMDS (4.00 eq, 55 mL, 71.7 mmol). The reaction mixture was stirred at room temperature for 16 h. The reaction mixture was poured into 200 mL of water, extracted with ethyl acetate (2×100 mL). The combined ethyl acetate phases were dried over sodium sulfate, filtered, and concentrated to obtain crude 5-((4,6-difluoro-1-((2-(trimethylsilyl)ethoxy)methyl)-1H-indol-5-yl)oxy)-2-fluorobenzimidamide (8 g). MS (ESI): m/z 436 [M+H]⁺.

### 2-[2-[2-[5-[4,6-Difluoro-]-(2-trimethylsilylethoxymethyl)indol-5-yl]oxy-2-fluoro-phenyl]-1H-imidazol-5-yl]ethyl]isoindoline-1,3-dione

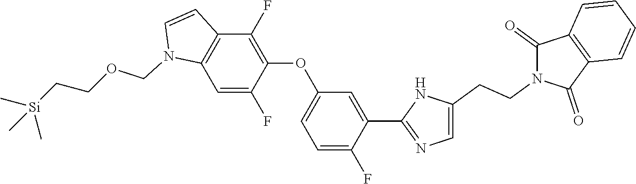 [SMILES: C[Si](C)(C)CCOCn1ccc2c(F)c(Oc3ccc(F)c(-c4ncc(CCN5C(=O)c6ccccc6C5=O)[nH]4)c3)c(F)cc21]

Step C: To a stirred solution of 5-[4,6-difluoro-1-(2-trimethylsilylethoxymethyl)indol-5-yl]oxy-2-fluoro-benzamidine (1.00 eq, 6.00 g, 13.8 mmol) in DMF (60 mL) was added at room temperature 2-(4-chloro-3-oxo-butyl)isoindoline-1,3-dione (1.50 eq, 5.20 g, 20.7 mmol) and NaHCO₃ (3.00 eq, 3.47 g, 41.3 mmol). The reaction mixture was heated at 70° C. for 16 h, then cooled to RT. The mixture was diluted with ethyl acetate (60 mL), washed with water (2×50 mL) and brine (50 mL). The organic phase was dried over sodium sulfate, filtered, and concentrated. The crude was purified by flash chromatography on silica gel, eluting with 10-60% ethyl acetate in hexanes, to obtain 2-[2-[2-[5-[4,6-difluoro-1-(2-trimethylsilylethoxymethyl)indol-5-yl]oxy-2-fluoro-phenyl]-1H-imidazol-5-yl]ethyl]isoindoline-1,3-dione (5.30 g, 7.62 mmol, 55% yield). as light-yellow semi solid. MS (ESI): m/z 633 [M+H]⁺.

### 2-[2-[5-[4,6-Difluoro-]-(2-trimethylsilylethoxymethyl)indol-5-yl]oxy-2-fluoro-phenyl]-1H-imidazol-5-yl]ethanamine

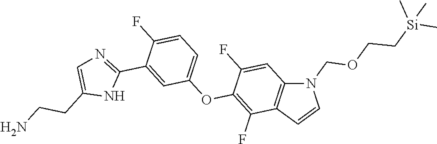 [SMILES: C[Si](C)(C)CCOCn1ccc2c(F)c(Oc3ccc(F)c(-c4ncc(CCN)[nH]4)c3)c(F)cc21]

Step D: To a stirred solution of 2-[2-[2-[5-[4,6-difluoro-1-(2-trimethylsilylethoxymethyl)indol-5-yl]oxy-2-fluoro-phenyl]-1H-imidazol-5-yl]ethyl]isoindoline-1,3-dione (1.00 eq, 4.50 g, 7.11 mmol) in ethanol (100 mL) was added at 25° C. hydrazine hydrate (1.75 eq, 0.60 mL, 12.4 mmol). The reaction mixture was heated at 80° C. for 4 h. The reaction mixture was cooled to RT, and the precipitate was removed by filtration. The filtrate was concentrated and co-distilled with ethyl acetate (2×20 mL) to obtain 2-[2-[5-[4,6-difluoro-1-(2-trimethylsilylethoxymethyl)indol-5-yl]oxy-2-fluoro-phenyl]-1H-imidazol-5-yl]ethanamine (3.00 g, 5.35 mmol, 75% yield) as a light-yellow semi solid. MS (ESI): m/z 503 [M+H]⁺.

### tert-Butyl (E)-3-(2-fluoro-3-formyl-phenyl)-2-methyl-prop-2-enoate and tert-butyl 2-(2-fluoro-3-formylbenzyl)acrylate

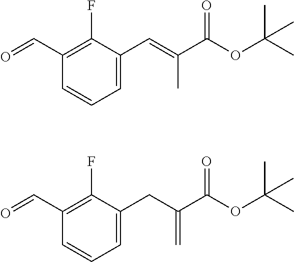 [SMILES: C/C(=C\c1cccc(C=O)c1F)C(=O)OC(C)(C)C ; C=C(Cc1cccc(C=O)c1F)C(=O)OC(C)(C)C]

Step E: A mixture of 3-bromo-2-fluoro-benzaldehyde (1.00 eq, 5.00 g, 24.6 mmol), tert-butyl 2-methylprop-2-enoate (3.00 eq, 12 mL, 73.9 mmol), TEA (5.00 eq, 17 mL, 123 mmol), tri(o-tolyl)phosphine (0.200 eq, 1.50 g, 4.93 mmol) and palladium(II) acetate (0.1000 eq, 0.55 g, 2.46 mmol) in degassed DMF (25 mL) was heated at 110° C. for 16 h under an argon atmosphere. After cooling, the reaction mixture was quenched with ice-cold water and extracted with EtOAc. The organic phase was washed with brine, dried over Na₂SO₄, and concentrated on a rotary evaporator. The residue was purified by silica gel column chromatography, eluting with a 5%-20% EtOAc in hexane, to give the desired product of tert-butyl (E)-3-(2-fluoro-3-formyl-phenyl)-2-methyl-prop-2-enoate (1.50 g, 5.68 mmol, 23% yield) as an off-white low-melting solid. LC-MS analysis reveals the presence of both the desired product and its isomer resulting from beta hydride elimination (tert-butyl 2-(2-fluoro-3-formylbenzyl)acrylate). MS (ESI): m/z 265 [M+H]⁺ observed for both product peaks.

### tert-Butyl (E)-3-[3-[2-[5-[4,6-difluoro-]-(2-trimethylsilylethoxymethyl)indol-5-yl]oxy-2-fluoro-phenyl]-4,5,6,7-tetrahydro-1H-imidazo[4,5-c]pyridin-4-yl]-2-fluoro-phenyl]-2-methyl-prop-2-enoate and tert-butyl 2-[[3-[2-[5-[4,6-difluoro-]-(2-trimethylsilylethoxymethyl)indol-5-yl]oxy-2-fluoro-phenyl]-4,5,6,7-tetrahydro-1H-imidazo[4,5-c]pyridin-4-yl]-2-fluoro-phenyl]methyl]prop-2-enoate

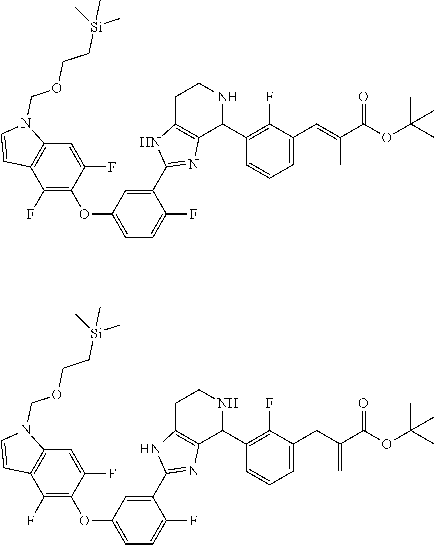 [SMILES: C/C(=C\c1cccc(C2NCCc3[nH]c(-c4cc(Oc5c(F)cc6c(ccn6COCC[Si](C)(C)C)c5F)ccc4F)nc32)c1F)C(=O)OC(C)(C)C ; C=C(Cc1cccc(C2NCCc3[nH]c(-c4cc(Oc5c(F)cc6c(ccn6COCC[Si](C)(C)C)c5F)ccc4F)nc32)c1F)C(=O)OC(C)(C)C]

Step F: To a stirred solution of 2-[2-[5-[4,6-difluoro-1-(2-trimethylsilylethoxymethyl)indol-5-yl]oxy-2-fluoro-phenyl]-1H-imidazol-5-yl]ethanamine (1.00 eq, 750 mg, 1.49 mmol) in ethanol (10 mL) was added TEA (3.00 eq, 0.62 mL, 4.48 mmol) followed by a mixture of tert-butyl (E)-3-(2-fluoro-3-formyl-phenyl)-2-methyl-prop-2-enoate (1.20 eq, 474 mg, 1.79 mmol) and tert-butyl 2-[(2-fluoro-3-formyl-phenyl)methyl]prop-2-enoate (1.00 eq, mg, ?). The resulting solution was heated at 70° C. for 22 h. The reaction mixture was cooled to room temperature, concentrated under reduced pressure. The residue was purified by flash chromatography on neutral alumina, eluting with 10-50% EtOAc in hexane, to give the desired product mixture of tert-butyl (E)-3-[3-[2-[5-[4,6-difluoro-1-(2-trimethylsilylethoxymethyl)indol-5-yl]oxy-2-fluoro-phenyl]-4,5,6,7-tetrahydro-1H-imidazo[4,5-c]pyridin-4-yl]-2-fluoro-phenyl]-2-methyl-prop-2-enoate and tert-butyl 2-[[3-[2-[5-[4,6-difluoro-1-(2-trimethylsilylethoxymethyl)indol-5-yl]oxy-2-fluoro-phenyl]-4,5,6,7-tetrahydro-1H-imidazo[4,5-c]pyridin-4-yl]-2-fluoro-phenyl]methyl]prop-2-enoate (504 mg, 0.673 mmol, 45% yield) as a pale-yellow, gummy solid. MS (ESI): m/z 749 [M+H]⁺.

### tert-Butyl 3-[3-[2-[5-[4,6-difluoro-]-(2-trimethylsilylethoxymethyl)indol-5-yl]oxy-2-fluoro-phenyl]-5-methyl-1,4,6,7-tetrahydroimidazo[4,5-c]pyridin-4-yl]-2-fluoro-phenyl]-2-methyl-propanoate

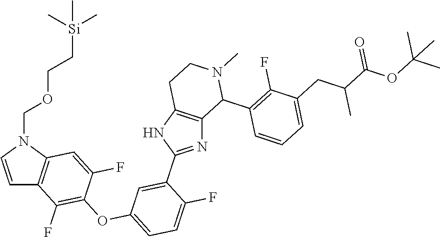 [SMILES: CC(Cc1cccc(C2c3nc(-c4cc(Oc5c(F)cc6c(ccn6COCC[Si](C)(C)C)c5F)ccc4F)[nH]c3CCN2C)c1F)C(=O)OC(C)(C)C]

Step G: To a solution of a mixture o tert-butyl (E)-3-[3-[2-[5-[4,6-difluoro-1-(2-trimethylsilylethoxymethyl)indol-5-yl]oxy-2-fluoro-phenyl]-4,5,6,7-tetrahydro-11H-imidazo[4,5-c]pyridin-4-yl]-2-fluoro-phenyl]-2-methyl-prop-2-enoate and tert-butyl 2-[[3-[2-[5-[4,6-difluoro-1-(2-trimethylsilylethoxymethyl)indol-5-yl]oxy-2-fluoro-phenyl]-4,5,6,7-tetrahydro-1H-imidazo[4,5-c]pyridin-4-yl]-2-fluoro-phenyl]methyl]prop-2-enoate (1.00 eq, 500 mg, 0.668 mmol) in methanol (25 mL) was added 38% aqueous formaldehyde solution (3.0 mL, 40.1 mmol) followed by Pd/C (10 wt. %, 150 mg, 1.41 mmol), and the suspension stirred under a hydrogen atmosphere at room temperature for 16 h. The reaction mixture was filtered through a Celite bed and the filtrate was concentrated. The crude product was dissolved in methanol (25 mL) and Pd/C (10 wt. %, 150 mg, 1.41 mmol) was added. Stirring under a hydrogen atmosphere at ambient temperature and pressure was continued for another 3 h. The reaction mixture was filtered through a Celite bed, and the bed was washed with MeOH. The combined filtrates were concentrated. The residue was purified by flash chromatography on neutral alumina, eluting with 50% EtOAc in hexane, to give the desired product tert-butyl 3-[3-[2-[5-[4,6-difluoro-1-(2-trimethylsilylethoxymethyl)indol-5-yl]oxy-2-fluoro-phenyl]-5-methyl-1,4,6,7-tetrahydroimidazo[4,5-c]pyridin-4-yl]-2-fluoro-phenyl]-2-methyl-propanoate (418 mg, 0.546 mmol, 82% yield) as an off-white solid. MS (ESI): m/z 765 [M+H]⁺.

### tert-Butyl 3-[3-[2-[5-[(4,6-difluoro-1H-indol-5-yl)oxy]-2-fluoro-phenyl]-5-methyl-1,4,6,7-tetrahydroimidazo[4,5-c]pyridin-4-yl]-2-fluoro-phenyl]-2-methyl-propanoate

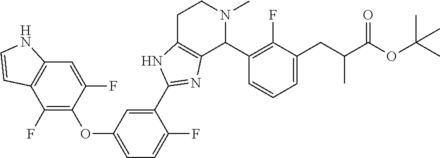 [SMILES: CC(Cc1cccc(C2c3nc(-c4cc(Oc5c(F)cc6[nH]ccc6c5F)ccc4F)[nH]c3CCN2C)c1F)C(=O)OC(C)(C)C]

Step H: To a stirred solution of tert-butyl 3-[3-[2-[5-[4,6-difluoro-1-(2-trimethylsilylethoxymethyl)indol-5-yl]oxy-2-fluoro-phenyl]-5-methyl-1,4,6,7-tetrahydroimidazo[4,5-c]pyridin-4-yl]-2-fluoro-phenyl]-2-methyl-propanoate (1.00 eq, 150 mg, 0.196 mmol) in THE (3 mL) was added at 0° C. tetrabutylammonium fluoride solution (1M in THF) (5.00 eq, 0.98 mL, 0.980 mmol) and then allowed to attain room temperature over 30 min. The reaction mixture was refluxed for 5 h, then cooled to rt, quenched with water, and extracted with EtOAc. The organic phase was washed with brine, dried over sodium sulphate, filtered, and concentrated. The crude product was purified by silica gel column chromatography, eluting with 20-60% EtOAc in hexane, to give the desired product tert-butyl 3-[3-[2-[5-[(4,6-difluoro-1H-indol-5-yl)oxy]-2-fluoro-phenyl]-5-methyl-1,4,6,7-tetrahydroimidazo[4,5-c]pyridin-4-yl]-2-fluoro-phenyl]-2-methyl-propanoate (25 mg, 0.0394 mmol, 20% yield) as an off-white gummy solid. MS (ESI): m/z 635 [M+H]⁺.

### 3-[3-[2-[5-[(4,6-Difluoro-1H-indol-5-yl)oxy]-2-fluoro-phenyl]-5-methyl-1,4,6,7-tetrahydroimidazo[4,5-c]pyridin-4-yl]-2-fluoro-phenyl]-2-methyl-propanoic acid

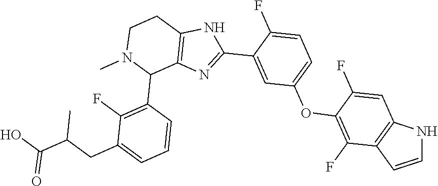 [SMILES: CC(Cc1cccc(C2c3nc(-c4cc(Oc5c(F)cc6[nH]ccc6c5F)ccc4F)[nH]c3CCN2C)c1F)C(=O)O]

Step I: To a stirred solution of tert-butyl 3-[3-[2-[5-[(4,6-difluoro-1H-indol-5-yl)oxy]-2-fluoro-phenyl]-5-methyl-1,4,6,7-tetrahydroimidazo[4,5-c]pyridin-4-yl]-2-fluoro-phenyl]-2-methyl-propanoate (1.00 eq, 20 mg, 0.0315 mmol) in DCM (2.5 mL) was added at 0° C. TFA (0.30 mL, 3.89 mmol). The reaction was stirred at rt for 3 hours and concentrated. The residue was dissolved in EtOAc, washed with 1% sodium bicarbonate aqueous solution and the aqueous back extracted with EtOAc. The combined organic extracts were washed with brine, dried over sodium sulfate, and concentrated. The residue was purified by preparative TLC developed with 10% MeOH in DCM to give the desired product of 3-[3-[2-[5-[(4,6-difluoro-1H-indol-5-yl)oxy]-2-fluoro-phenyl]-5-methyl-1,4,6,7-tetrahydroimidazo[4,5-c]pyridin-4-yl]-2-fluoro-phenyl]-2-methyl-propanoic acid (2.2 mg, 0.00352 mmol, 12% yield) as an off-white solid. MS (ESI): m/z 579 [M+H]⁺.

### Example 12. Synthesis of 3-[3-[5-(2,2-difluoroethyl)-2-[5-[(4,6-difluoro-1H-indol-5-yl)oxy]-2-fluoro-phenyl]-1,4,6,7-tetrahydroimidazo[4,5-c]pyridin-4-yl]-2-fluoro-phenyl]propanoic acid

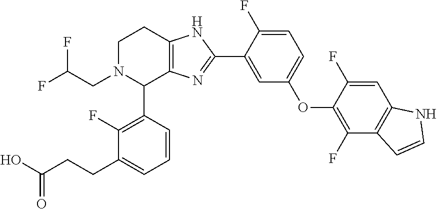 [SMILES: O=C(O)CCc1cccc(C2c3nc(-c4cc(Oc5c(F)cc6[nH]ccc6c5F)ccc4F)[nH]c3CCN2CC(F)F)c1F]

### Ethyl 3-[3-[2-[5-[4,6-difluoro-]-(2-trimethylsilylethoxymethyl)indol-5-yl]oxy-2-fluoro-phenyl]-4,5,6,7-tetrahydro-3H-imidazo[4,5-c]pyridin-4-yl]-2-fluoro-phenyl]propanoate

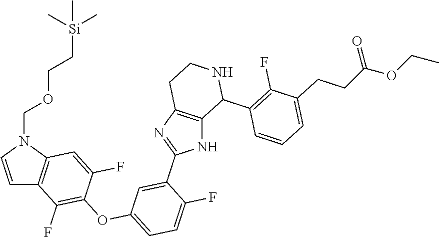 [SMILES: CCOC(=O)CCc1cccc(C2NCCc3nc(-c4cc(Oc5c(F)cc6c(ccn6COCC[Si](C)(C)C)c5F)ccc4F)[nH]c32)c1F]

Step A: To a stirred solution of 2-[2-[5-[4,6-difluoro-1-(2-trimethylsilylethoxymethyl)indol-5-yl]oxy-2-fluoro-phenyl]-1H-imidazol-5-yl]ethanamine (Step D, Example 11) (1.00 eq, 600 mg, 1.19 mmol) and ethyl 3-(2-fluoro-3-formyl-phenyl)propanoate (1.00 eq, 268 mg, 1.19 mmol) in ethanol (12 mL) was added TEA (3.00 eq, 0.50 mL, 3.58 mmol). The reaction mixture was stirred at 70° C. for 16 h and concentrated. The crude was dissolved in DCM (10 mL), washed with water (10 mL), brine (10 mL), dried over sodium sulfate, filtered, and concentrated. The crude product was purified by silica gel column chromatography, eluting with 0-5% methanol in DCM, to obtain ethyl 3-[3-[2-[5-[4,6-difluoro-1-(2-trimethylsilylethoxymethyl)indol-5-yl]oxy-2-fluoro-phenyl]-4,5,6,7-tetrahydro-3H-imidazo[4,5-c]pyridin-4-yl]-2-fluoro-phenyl]propanoate (500 mg, 0.684 mmol, 57% yield) as a light-yellow solid. MS (ESI): m/z 709 [M+H]⁺.

### Ethyl 3-[3-[5-(2,2-difluoroethyl)-2-[5-[4,6-difluoro-]-(2-trimethylsilylethoxymethyl)indol-5-yl]oxy-2 fluoro-phenyl]-1,4,6,7-tetrahydroimidazo[4,5-c]pyridin-4-yl]-2-fluoro-phenyl]propanoate

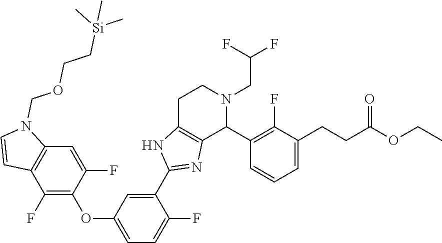 [SMILES: CCOC(=O)CCc1cccc(C2c3nc(-c4cc(Oc5c(F)cc6c(ccn6COCC[Si](C)(C)C)c5F)ccc4F)[nH]c3CCN2CC(F)F)c1F]

Step B: To a solution of ethyl 3-[3-[2-[5-[4,6-difluoro-1-(2-trimethylsilylethoxymethyl)indol-5-yl]oxy-2-fluoro-phenyl]-4,5,6,7-tetrahydro-1H-imidazo[4,5-c]pyridin-4-yl]-2-fluoro-phenyl]propanoate (1.00 eq, 350 mg, 0.494 mmol) in DMF (6 mL) were added at rt TEA (3.00 eq, 0.21 mL, 1.48 mmol) and 1,1-difluoro-2-iodo-ethane (1.20 eq, 114 mg, 0.593 mmol). The reaction mixture was stirred at 80° C. for 16 h. The reaction mixture was cooled to RT, diluted with ethyl acetate (15 mL), and washed with water (20 mL), and brine (30 mL). The organic phase was dried over sodium sulfate, filtered, and concentrated. The crude product was purified via flash chromatography on silica gel, eluting with 0-50% ethyl acetate in hexanes, to obtain ethyl 3-[3-[5-(2,2-difluoroethyl)-2-[5-[4,6-difluoro-1-(2-trimethylsilylethoxymethyl)indol-5-yl]oxy-2-fluoro-phenyl]-1,4,6,7-tetrahydroimidazo[4,5-c]pyridin-4-yl]-2-fluoro-phenyl]propanoate (200 mg, 0.241 mmol, 48% yield) as a light-yellow solid. MS (ESI): m/z 773 [M+H]⁺.

### 3-[3-[5-(2,2-Difluoroethyl)-2-[5-[(4,6-difluoro-1H-indol-5-yl)oxy]-2-fluoro-phenyl]-1,4,6,7-tetrahydroimidazo[4,5-c]pyridin-4-yl]-2-fluoro-phenyl]propanoic acid

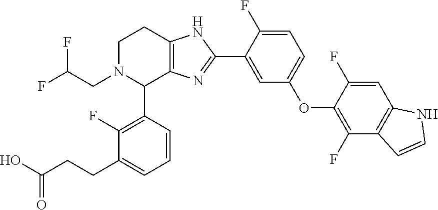 [SMILES: O=C(O)CCc1cccc(C2c3nc(-c4cc(Oc5c(F)cc6[nH]ccc6c5F)ccc4F)[nH]c3CCN2CC(F)F)c1F]

Step C: To a stirred solution of ethyl 3-[3-[5-(2,2-difluoroethyl)-2-[5-[4,6-difluoro-1-(2-trimethylsilylethoxymethyl)indol-5-yl]oxy-2-fluoro-phenyl]-1,4,6,7-tetrahydroimidazo[4,5-c]pyridin-4-yl]-2-fluoro-phenyl]propanoate (1.00 eq, 160 mg, 0.207 mmol) in THE (2 mL) was added at room temperature tetrabutylammonium fluoride (10.0 eq, 2.1 mL, 2.07 mmol). The reaction mixture was heated at 60° C. in a sealed tube for 16 h. The reaction was cooled to RT, concentrated under reduced pressure, followed by addition of water. The solid precipitate was isolated by filtration. The solid was purified via column chromatography on silica gel, eluting with 20-60% ethyl acetate in hexanes, to afford 50 mg of partially purified desired product. This material was purified by prep-HPLC, followed by prep-TLC (silica gel developing with EtOAc), to obtain 3-[3-[5-(2,2-difluoroethyl)-2-[5-[(4,6-difluoro-1H-indol-5-yl)oxy]-2-fluoro-phenyl]-1,4,6,7-tetrahydroimidazo[4,5-c]pyridin-4-yl]-2-fluoro-phenyl]propanoic acid (4.2 mg, 0.00650 mmol, 3% yield) as a white solid. MS (ESI): m/z 615 [M+H]⁺.

### Example 13. Synthesis of ethyl 3-[2-fluoro-3-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]phenyl]propanoate

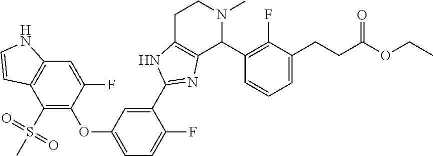 [SMILES: CCOC(=O)CCc1cccc(C2c3nc(-c4cc(Oc5c(F)cc6[nH]ccc6c5S(C)(=O)=O)ccc4F)[nH]c3CCN2C)c1F]

### Ethyl (E)-3-[2-fluoro-3-[2-[2-fluoro-5-[6-fluoro-4-methylsulfonyl-1-(p-tolylsulfonyl)indol-5-yl]oxy-phenyl]-4,5,6,7-tetrahydro-1H-imidazo[4,5-c]pyridin-4-yl]phenyl]prop-2-enoate

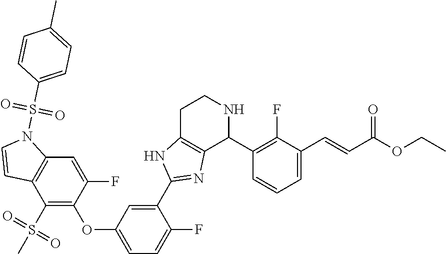 [SMILES: CCOC(=O)/C=C/c1cccc(C2NCCc3[nH]c(-c4cc(Oc5c(F)cc6c(ccn6S(=O)(=O)c6ccc(C)cc6)c5S(C)(=O)=O)ccc4F)nc32)c1F]

Step A: To flame dried 4 Å molecular sieves was added at RT a solution of 2-[2-[2-fluoro-5-[6-fluoro-4-methylsulfonyl-1-(p-tolylsulfonyl)indol-5-yl]oxy-phenyl]-1H-imidazol-4-yl]ethanamine (1.00 eq, 1.20 g, 2.05 mmol) (Step E, Example 7) in ethanol (5 mL), followed by ethyl (E)-3-(2-fluoro-3-formyl-phenyl)prop-2-enoate (1.20 eq, 0.55 g, 2.45 mmol) and TEA (3.00 eq, 0.86 mL, 6.14 mmol). The reaction was stirred at 70-80° C. for 48 h and concentrated. The crude was purified by flash chromatography on silica gel, eluting with 2-4% of MeOH in DCM, to afford ethyl (E)-3-[2-fluoro-3-[2-[2-fluoro-5-[6-fluoro-4-methylsulfonyl-1-(p-tolylsulfonyl)indol-5-yl]oxy-phenyl]-4,5,6,7-tetrahydro-1H-imidazo[4,5-c]pyridin-4-yl]phenyl]prop-2-enoate (1.00 g, 1.26 mmol, 62% yield) as a pale brown solid. MS (ESI): m/z 791 [M+H]⁺.

### Ethyl 3-[2-fluoro-3-[2-[2-fluoro-5-[6-fluoro-4-methylsulfonyl-1-(p-tolylsulfonyl)indol-5-yl]oxy-phenyl]-5-methyl-1,4,6,7-tetrahydroimidazo[4,5-c]pyridin-4-yl]phenyl]propanoate

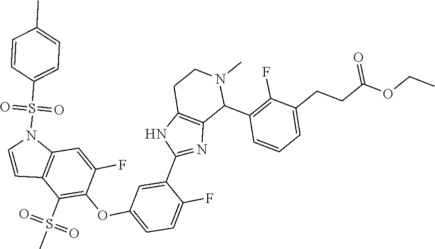 [SMILES: CCOC(=O)CCc1cccc(C2c3nc(-c4cc(Oc5c(F)cc6c(ccn6S(=O)(=O)c6ccc(C)cc6)c5S(C)(=O)=O)ccc4F)[nH]c3CCN2C)c1F]

Step B: To a stirred solution of ethyl (E)-3-[2-fluoro-3-[2-[2-fluoro-5-[6-fluoro-4-methylsulfonyl-1-(p-tolylsulfonyl)indol-5-yl]oxy-phenyl]-4,5,6,7-tetrahydro-1H-imidazo[4,5-c]pyridin-4-yl]phenyl]prop-2-enoate (1.00 eq, 500 mg, 0.632 mmol) in methanol (10 mL) was added at RT aqueous formaldehyde (37% w/v, 1.50 eq, mg, 0.948 mmol) and Pd/C (10% wt., 100 mg). The reaction was stirred for 6 h under a hydrogen atmosphere at ambient temperature. The reaction mixture was filtered through a Celite pad, and the pad was washed with methanol. The combined filtrates were concentrated under reduced pressure to afford ethyl 3-[2-fluoro-3-[2-[2-fluoro-5-[6-fluoro-4-methylsulfonyl-1-(p-tolylsulfonyl)indol-5-yl]oxy-phenyl]-5-methyl-1,4,6,7-tetrahydroimidazo[4,5-c]pyridin-4-yl]phenyl]propanoate (300 mg, 0.372 mmol, 59% yield) as an off-white solid. MS (ESI): m/z 807 [M+H]⁺.

### Ethyl 3-[2-fluoro-3-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]phenyl]propanoate

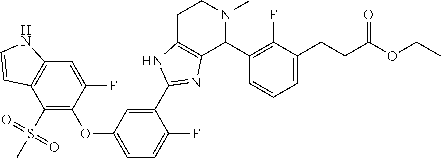 [SMILES: CCOC(=O)CCc1cccc(C2c3nc(-c4cc(Oc5c(F)cc6[nH]ccc6c5S(C)(=O)=O)ccc4F)[nH]c3CCN2C)c1F]

Step C: To a chilled (0° C.) and stirred solution of ethyl 3-[2-fluoro-3-[2-[2-fluoro-5-[6-fluoro-4-methylsulfonyl-1-(p-tolylsulfonyl)indol-5-yl]oxy-phenyl]-5-methyl-1,4,6,7-tetrahydroimidazo[4,5-c]pyridin-4-yl]phenyl]propanoate (1.00 eq, 40 mg, 0.0496 mmol) in THE was added NaH (60% dispersion in oil, 10.0 eq, 14 mg, 0.496 mmol). The reaction mixture was stirred for 2 h at 0° C. The reaction was quenched with water and extracted with EtOAc. The organic phase was dried over Na₂SO₄, filtered, and concentrated under reduced pressure. The crude was purified by prep-TLC (silica gel, developed with 10% MeOH in DCM), to afford ethyl 3-[2-fluoro-3-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]phenyl]propanoate (5.0 mg, 0.00698 mmol, 14% yield) as an off-white solid. MS (ESI): m/z 653 [M+H]⁺.

### Example 14. Synthesis of 3-[2-fluoro-3-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]phenyl]propanoic acid

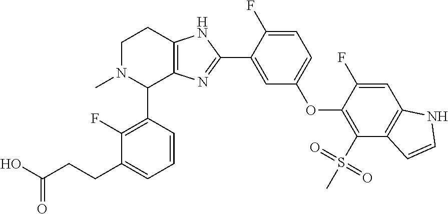 [SMILES: CN1CCc2[nH]c(-c3cc(Oc4c(F)cc5[nH]ccc5c4S(C)(=O)=O)ccc3F)nc2C1c1cccc(CCC(=O)O)c1F]

Step A: To a stirred solution of ethyl 3-[2-fluoro-3-[2-[2-fluoro-5-[6-fluoro-4-methylsulfonyl-1-(p-tolylsulfonyl)indol-5-yl]oxy-phenyl]-5-methyl-1,4,6,7-tetrahydroimidazo[4,5-c]pyridin-4-yl]phenyl]propanoate (1.00 eq, 100 mg, 0.124 mmol) in methanol (1 mL) was added at rt a solution of NaOH (2.00 eq, 9.9 mg, 0.248 mmol) in water (0.5 mL). The mixture was stirred for 16 h at rt. The reaction was neutralized (pH-7) by using aqueous 1N HCl and concentrated under reduced pressure. The crude was dissolved in methanol and filtered through a Celite pad. The filtrates were concentrated. The solid was washed with water, dried under high vacuo to afford 3-[2-fluoro-3-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]phenyl]propanoic acid (5.0 mg, 0.00721 mmol, 6% yield) as an off-white solid. MS (ESI): m/z 625 [M+H]⁺.

### Example 15. Synthesis of ethyl 3-[3-[5-(2,2-difluoroethyl)-2-[2-fluoro-5-[(6-fluoro-4-methylsulfonyl-1H-indol-5-yl)oxy]phenyl]-1,4,6,7-tetrahydroimidazo[4,5-c]pyridin-4-yl]-2-fluoro-phenyl]propanoate

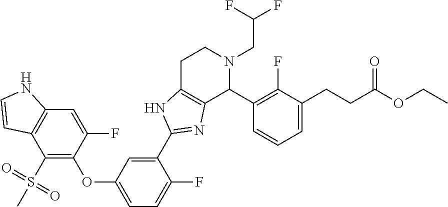 [SMILES: CCOC(=O)CCc1cccc(C2c3nc(-c4cc(Oc5c(F)cc6[nH]ccc6c5S(C)(=O)=O)ccc4F)[nH]c3CCN2CC(F)F)c1F]

### Ethyl 3-[2-fluoro-3-[2-[2-fluoro-5-[6-fluoro-4-methylsulfonyl-1-(p-tolylsulfonyl)indol-5-yl]oxy-phenyl]-4,5,6,7-tetrahydro-1H-imidazo[4,5-c]pyridin-4-yl]phenyl]propanoate

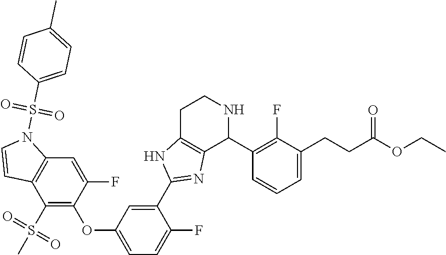 [SMILES: CCOC(=O)CCc1cccc(C2NCCc3[nH]c(-c4cc(Oc5c(F)cc6c(ccn6S(=O)(=O)c6ccc(C)cc6)c5S(C)(=O)=O)ccc4F)nc32)c1F]

Step A: To flame dried 4 Å molecular sieves was added at RT a solution of 2-[2-[2-fluoro-5-[6-fluoro-4-methylsulfonyl-1-(p-tolylsulfonyl)indol-5-yl]oxy-phenyl]-1H-imidazol-4-yl]ethanamine (Step E, Example 7) (1.00 eq, 300 mg, 0.511 mmol) in ethanol (5 mL), followed by the addition of ethyl 3-(2-fluoro-3-formyl-phenyl)propanoate (1.20 eq, 138 mg, 0.614 mmol) and TEA (3.00 eq, 0.22 mL, 1.53 mmol). The reaction mass was stirred at 70-80° C. for 48 h. The molecular sieves were removed by filtration and the filtrate was concentrated under reduced pressure. The residue was purified by flash chromatography on silica gel eluting with 2-4% MeOH in DCM to afford ethyl 3-[2-fluoro-3-[2-[2-fluoro-5-[6-fluoro-4-methylsulfonyl-1-(p-tolylsulfonyl)indol-5-yl]oxy-phenyl]-4,5,6,7-tetrahydro-1H-imidazo[4,5-c]pyridin-4-yl]phenyl]propanoate (100 mg, 0.126 mmol, 25% yield) as a pale-brown solid. MS (ESI): m/z 793 [M+H]⁺.

### Ethyl 3-[3-[5-(2,2-difluoroethyl)-2-[2-fluoro-5-[6-fluoro-4-methylsulfonyl-]-(p-tolylsulfonyl) indol-5-yl]oxy-phenyl]-1,4,6,7-tetrahydroimidazo[4,5-c]pyridin-4-yl]-2-fluoro-phenyl]propanoate

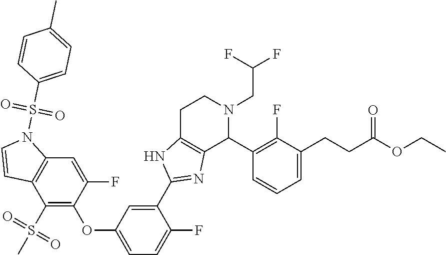 [SMILES: CCOC(=O)CCc1cccc(C2c3nc(-c4cc(Oc5c(F)cc6c(ccn6S(=O)(=O)c6ccc(C)cc6)c5S(C)(=O)=O)ccc4F)[nH]c3CCN2CC(F)F)c1F]

Step B: To a stirred solution of ethyl 3-[2-fluoro-3-[2-[2-fluoro-5-[6-fluoro-4-methylsulfonyl-1-(p-tolylsulfonyl)indol-5-yl]oxy-phenyl]-4,5,6,7-tetrahydro-1H-imidazo[4,5-c]pyridin-4-yl]phenyl]propanoate (1.00 eq, 100 mg, 0.126 mmol) in DMF (0.5 mL) was added at RT 1,1-difluoro-2-iodo-ethane (1.20 eq, 29 mg, 0.151 mmol) and TEA (3.00 eq, 38 mg, 0.378 mmol). The mixture was stirred for 48 h at 80° C. The reaction was diluted with water and extracted with EtOAc. The organic phase was dried over anhydrous Na₂SO₄, filtered, and concentrated under reduced pressure. The crude was purified by flash chromatography on silica, eluting with 3-5% of methanol in EtOAc, to afford ethyl 3-[3-[5-(2,2-difluoroethyl)-2-[2-fluoro-5-[6-fluoro-4-methylsulfonyl-1-(p-tolylsulfonyl)indol-5-yl]oxy-phenyl]-1,4,6,7-tetrahydroimidazo[4,5-c]pyridin-4-yl]-2-fluoro-phenyl]propanoate (10 mg, 0.0117 mmol, 9% yield) as a pale brown solid. MS (ESI): m/z 857 [M+H]⁺.

### Ethyl 3-[3-[5-(2,2-difluoroethyl)-2-[2-fluoro-5-[(6-fluoro-4-methylsulfonyl-1H-indol-5-yl)oxy]phenyl]-1,4,6,7-tetrahydroimidazo[4,5-c]pyridin-4-yl]-2-fluoro-phenyl]propanoate

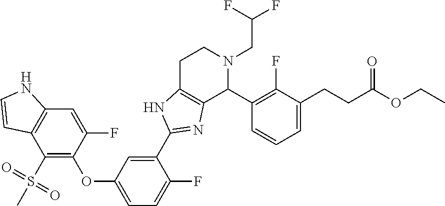 [SMILES: CCOC(=O)CCc1cccc(C2c3nc(-c4cc(Oc5c(F)cc6[nH]ccc6c5S(C)(=O)=O)ccc4F)[nH]c3CCN2CC(F)F)c1F]

Step C: To a stirred and chilled (0° C.) solution of ethyl 3-[3-[5-(2,2-difluoroethyl)-2-[2-fluoro-5-[6-fluoro-4-methylsulfonyl-1-(p-tolylsulfonyl)indol-5-yl]oxy-phenyl]-1,4,6,7-tetrahydroimidazo[4,5-c]pyridin-4-yl]-2-fluoro-phenyl]propanoate (1.00 eq, 20 mg, 0.0233 mmol) in THE was added NaH (60% dispersion in oil, 10.0 eq, 2.6 mg, 0.233 mmol). The reaction was stirred for 6 h at 0° C. The reaction mixture was partitioned between EtOAc and water. The organic phase was dried over Na₂SO₄, filtered, and concentrated under reduced pressure. The residue was purified by prep-TLC (silica gel, eluting with 10% MeOH in DCM) to afford ethyl 3-[3-[5-(2,2-difluoroethyl)-2-[2-fluoro-5-[(6-fluoro-4-methylsulfonyl-1H-indol-5-yl)oxy]phenyl]-1,4,6,7-tetrahydroimidazo[4,5-c]pyridin-4-yl]-2-fluoro-phenyl]propanoate (5.0 mg, 0.00677 mmol, 29% yield) as an off-white solid. MS (ESI): m/z 703 [M+H]⁺.

### Example 16. Synthesis of 3-[3-[5-(2,2-difluoroethyl)-2-[2-fluoro-5-[(6-fluoro-4-methylsulfonyl-1H-indol-5-yl)oxy]phenyl]-1,4,6,7-tetrahydroimidazo[4,5-c]pyridin-4-yl]-2-fluoro-phenyl]propanoic acid

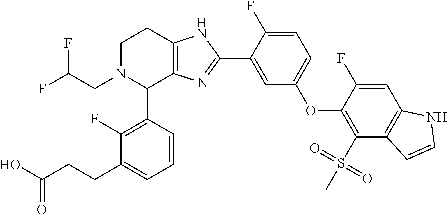 [SMILES: CS(=O)(=O)c1c(Oc2ccc(F)c(-c3nc4c([nH]3)CCN(CC(F)F)C4c3cccc(CCC(=O)O)c3F)c2)c(F)cc2[nH]ccc12]

To a stirred solution of ethyl 3-[3-[5-(2,2-difluoroethyl)-2-[2-fluoro-5-[6-fluoro-4-methylsulfonyl-1-(p-tolylsulfonyl)indol-5-yl]oxy-phenyl]-1,4,6,7-tetrahydroimidazo[4,5-c]pyridin-4-yl]-2-fluoro-phenyl]propanoate (Example 15) (1.00 eq, 50 mg, 0.0585 mmol) in methanol (3 mL) was added at RT and under nitrogen 6 N aqueous NaOH (0.5 ml). The mixture was stirred at RT for 16 h, the solids filtered off, and the filtrate concentrated. The crude was dissolved in EtOAc and precipitated with n-pentane to afford 3-[3-[5-(2,2-difluoroethyl)-2-[2-fluoro-5-[(6-fluoro-4-methylsulfonyl-1H-indol-5-yl)oxy]phenyl]-1,4,6,7-tetrahydroimidazo[4,5-c]pyridin-4-yl]-2-fluoro-phenyl]propanoic acid (35 mg, 0.0519 mmol, 89% yield) as an off-white solid. MS (ESI): m/z 675 [M+H]⁺.

### Example 17. Synthesis of 3-[3-[5-(2,2-difluoroethyl)-2-[2-fluoro-5-[(6-fluoro-4-methylsulfonyl-1H-indol-5-yl)oxy]phenyl]-1,4,6,7-tetrahydroimidazo[4,5-c]pyridin-4-yl]-2-fluoro-phenyl]-N-methyl-propanamide

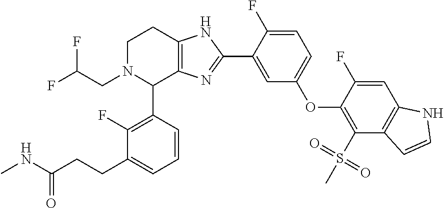 [SMILES: CNC(=O)CCc1cccc(C2c3nc(-c4cc(Oc5c(F)cc6[nH]ccc6c5S(C)(=O)=O)ccc4F)[nH]c3CCN2CC(F)F)c1F]

To a stirred solution of 3-[3-[5-(2,2-difluoroethyl)-2-[2-fluoro-5-[(6-fluoro-4-methylsulfonyl-1H-indol-5-yl)oxy]phenyl]-1,4,6,7-tetrahydroimidazo[4,5-c]pyridin-4-yl]-2-fluoro-phenyl]propanoic acid (Example 16) (1.00 eq, 35 mg, 0.0519 mmol) in DMF (3 mL) was added at 0° C. a solution of methylamine in THE (7%, 2.00 eq), HATU (1.20 eq, 30 mg, 0.0623 mmol), and DIPEA (3.00 eq, 0.50 mg, 0.156 mmol). The reaction was stirred for 16 h at RT. The reaction mixture was diluted with water, and extracted with EtOAc. The organic extract was dried over anhydrous Na₂SO₄, filtered, and concentrated under reduced pressure. The crude was purified by prep-TLC (silica gel), eluting with 10% MeOH in DCM, to afford 3-[3-[5-(2,2-difluoroethyl)-2-[2-fluoro-5-[(6-fluoro-4-methylsulfonyl-1H-indol-5-yl)oxy]phenyl]-1,4,6,7-tetrahydroimidazo[4,5-c]pyridin-4-yl]-2-fluoro-phenyl]-N-methyl-propanamide (6.1 mg, 0.00849 mmol, 16% yield) as an off-white solid. MS (ESI): m/z 688 [M+H]⁺.

### Example 18. Synthesis of ethyl 3-[5-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]-2-thienyl]propanoate

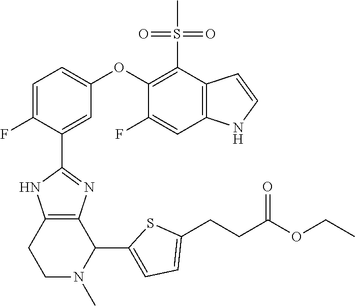 [SMILES: CCOC(=O)CCc1ccc(C2c3nc(-c4cc(Oc5c(F)cc6[nH]ccc6c5S(C)(=O)=O)ccc4F)[nH]c3CCN2C)s1]

### Ethyl 3-[5-[2-[2-fluoro-5-[6-fluoro-4-methylsulfonyl-]-(p-tolylsulfonyl)indol-5-yl]oxy-phenyl]-4,5,6,7-tetrahydro-1H-imidazo[4,5-c]pyridin-4-yl]-2-thienyl]propanoate

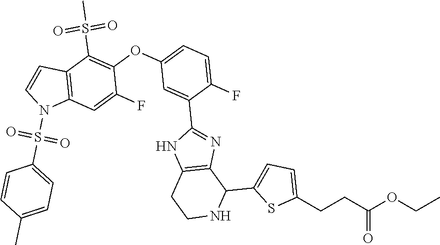 [SMILES: CCOC(=O)CCc1ccc(C2NCCc3[nH]c(-c4cc(Oc5c(F)cc6c(ccn6S(=O)(=O)c6ccc(C)cc6)c5S(C)(=O)=O)ccc4F)nc32)s1]

Step A: To a stirred solution of 2-[2-[2-fluoro-5-[6-fluoro-4-methylsulfonyl-1-(p-tolylsulfonyl)indol-5-yl]oxy-phenyl]-1H-imidazol-4-yl]ethanamine (1.00 eq, 200 mg, 0.341 mmol) (Step E, Example 7) in ethanol (5 mL) was added at rt ethyl 3-(5-formyl-2-thienyl)propanoate (1.20 eq, 0.087 g, 0.409 mmol) followed by TEA (3.00 eq, 103 mg, 1.02 mmol). The reaction mixture was stirred at 70° C. for 48 h, cooled to rt and concentrated. The residue was purified by silica gel column chromatography, eluting with 1-5% MeOH in DCM, to give the desired product, ethyl 3-[5-[2-[2-fluoro-5-[6-fluoro-4-methylsulfonyl-1-(p-tolylsulfonyl)indol-5-yl]oxy-phenyl]-4,5,6,7-tetrahydro-1H-imidazo[4,5-c]pyridin-4-yl]-2-thienyl]propanoate (108 mg, 0.138 mmol, 41% yield) as a pale yellowish-brown solid. MS (ESI): m/z 781 [M+H]⁺.

### Ethyl 3-[5-[2-[2-fluoro-5-[6-fluoro-4-methylsulfonyl-]-(p-tolylsulfonyl)indol-5-yl]oxy-phenyl]-5-methyl-1,4,6,7-tetrahydroimidazo[4,5-c]pyridin-4-yl]-2-thienyl]propanoate

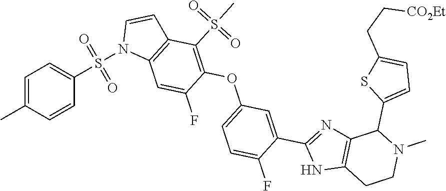

Step B: To a stirred solution of ethyl 3-[5-[2-[2-fluoro-5-[6-fluoro-4-methylsulfonyl-1-(p-tolylsulfonyl)indol-5-yl]oxy-phenyl]-4,5,6,7-tetrahydro-1H-imidazo[4,5-c]pyridin-4-yl]-2-thienyl]propanoate (1.00 eq, 100 mg, 0.128 mmol) in methanol (5 mL) was added at rt aqueous formaldehyde solution (38% w/v, 1.0 mL) followed by sodium cyanoborohydride (1.00 eq, 8.1 mg, 0.129 mmol). After stirring for 1 hour at rt, acetic acid (1.00 eq, 0.20 mL, 3.49 mmol) was added, and the reaction was stirred for another 20 hours. After the addition of 10 mL of water and adjustment of the pH to about 9 with 6 N aqueous sodium hydroxide, the mixture was extracted with EtOAc. The combined organic extracts were washed with water, brine, dried over sodium sulfate, filtered, and concentrated. The crude product was purified by silica gel column chromatography, eluting with 20-80% EtOAc in hexane, to give the desired product, ethyl 3-[5-[2-[2-fluoro-5-[6-fluoro-4-methylsulfonyl-1-(p-tolylsulfonyl)indol-5-yl]oxy-phenyl]-5-methyl-1,4,6,7-tetrahydroimidazo[4,5-c]pyridin-4-yl]-2-thienyl]propanoate (51 mg, 0.0642 mmol, 50% yield) as a pale-yellow, gummy solid. MS (ESI): m/z 795 [M+H]⁺.

### Ethyl 3-[5-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]-2-thienyl]propanoate

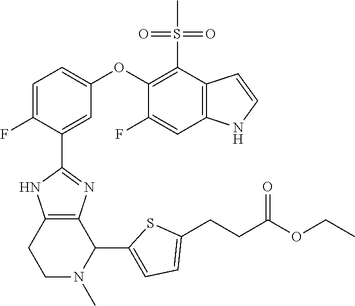 [SMILES: CCOC(=O)CCc1ccc(C2c3nc(-c4cc(Oc5c(F)cc6[nH]ccc6c5S(C)(=O)=O)ccc4F)[nH]c3CCN2C)s1]

Step C: To a stirred solution of ethyl 3-[5-[2-[2-fluoro-5-[6-fluoro-4-methylsulfonyl-1-(p-tolylsulfonyl)indol-5-yl]oxy-phenyl]-5-methyl-1,4,6,7-tetrahydroimidazo[4,5-c]pyridin-4-yl]-2-thienyl]propanoate (1.00 eq, 30 mg, 0.0377 mmol) in THE (2.5 mL) was added at 0° C. sodium hydride (7.5 mg, 0.188 mmol). After stirring for 3 hours at 0° C., an additional 7.5 mg of sodium hydride were added, and stirring continued for another 3 hours at 0° C. The reaction mixture was partitioned between EtOAc and water. The separated organic phase was dried over sodium sulphate and concentrated. The crude was purified by preparative TLC (silica gel), eluting with 7.5% MeOH in DCM, to give 11 mg of the partially purified desired product. This material was triturated with n-pentane and dried under vacuum to afford pure ethyl 3-[5-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]-2-thienyl]propanoate (4.5 mg, 0.00670 mmol, 18%) as an off-white solid. MS (ESI): m/z 641 [M+H_{1+.}

### Example 19. Synthesis of 3-[5-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]-2-thienyl]propanoic acid

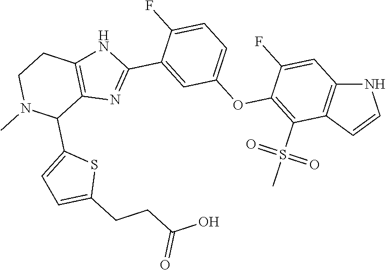 [SMILES: CN1CCc2[nH]c(-c3cc(Oc4c(F)cc5[nH]ccc5c4S(C)(=O)=O)ccc3F)nc2C1c1ccc(CCC(=O)O)s1]

To a stirred solution of ethyl 3-[5-[2-[2-fluoro-5-[6-fluoro-4-methylsulfonyl-1-(p-tolylsulfonyl)indol-5-yl]oxy-phenyl]-5-methyl-1,4,6,7-tetrahydroimidazo[4,5-c]pyridin-4-yl]-2-thienyl]propanoate (Example 14) (1.00 eq, 25 mg, 0.0315 mmol) in methanol (2.5 mL) was added at 0° C. NaOH (6N aqueous solution, 10.0 eq, 0.50 mL, 3.00 mmol). The reaction was stirred at room temperature for 20 h and concentrated. This crude product was purified by silica gel preparative TLC, eluting with 15% MeOH in DCM. The partially purified material was triturated with water, rinsed with n-pentane, and dried under vacuum to give the desired 3-[5-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]-2-thienyl]propanoic acid (4.4 mg, 0.00684 mmol, 21% yield) as an off-white solid. MS (ESI): m/z 613 [M+H]⁺.

### Example 20. Synthesis of 3-[3-[2-[5-[(4,6-difluoro-1H-indol-5-yl)oxy]-2-fluoro-phenyl]-5-methyl-1,4,6,7-tetrahydroimidazo[4,5-c]pyridin-4-yl]-2-fluoro-phenyl]propane-1,2-diol

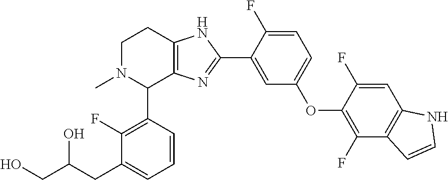 [SMILES: CN1CCc2[nH]c(-c3cc(Oc4c(F)cc5[nH]ccc5c4F)ccc3F)nc2C1c1cccc(CC(O)CO)c1F]

### (3-Allyl-2-fluoro-phenyl)methanol

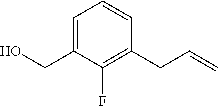

Step A: To a stirred solution of (3-bromo-2-fluoro-phenyl)methanol (1.00 eq, 100 mg, 0.488 mmol) in 1,4-dioxane (2.5 mL) and water (0.5 mL) were added at rt and under an argon atmosphere 2-allyl-4,4,5,5-tetramethyl-1,3,2-dioxaborolane (5.00 eq, 410 mg, 2.44 mmol), [1,1′-bis(diphenylphosphino)ferrocene]dichloropalladium(II), complex with dichloromethane (0.1000 eq, 40 mg, 0.0488 mmol), and potassium carbonate (3.00 eq, 202 mg, 1.46 mmol). The reaction mixture was stirred at 100° C. for 16 h. The reaction mixture was allowed to cooled to room temperature and partitioned between EtOAc and water. The separated organic phase was washed with brine, dried over sodium sulphate, filtered, and concentrated under reduced pressure. The residue was purified by silica gel column chromatography, eluting with 5-10% EtOAc in hexane, to give the desired product (3-allyl-2-fluoro-phenyl)methanol (42 mg, 0.253 mmol, 52% yield) as colorless, thick liquid. ¹H NMR (300 MHz, DMSO-d₆) δ 7.35-7.29 (m, 1H), 7.18-7.09 (m, 2H), 6.01-5.87 (m, 1H), 5.21 (t, 1H), 5.07 (t, 1H), 5.05-5.01 (m, 1H), 4.53 (d, 2H).

### 3-Allyl-2-fluoro-benzaldehyde

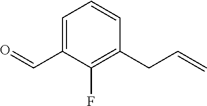

Step B: To a stirred solution of (3-allyl-2-fluoro-phenyl)methanol (1.00 eq, 40 mg, 0.241 mmol) in DCM (2 mL) was added at 0° C. Dess-Martin periodinane (1.50 eq, 153 mg, 0.361 mmol). The mixture was then stirred at room temperature for 5 h, diluted with DCM, and filtered through a Celite bed. The filtrate was washed with saturated aqueous sodium bicarbonate solution, brine, dried over sodium sulphate, filtered, and concentrated under reduced pressure. The crude was purified by silica gel column chromatography, eluting with 5% EtOAc in hexane, to give 3-allyl-2-fluoro-benzaldehyde (33 mg, 0.201 mmol, 83% yield) as an off-white solid. ¹H NMR (300 MHz, DMSO-d₆) δ 10.24 (s, 1H), 7.72 (td, 1H), 7.63 (td, 1H), 7.34 (t, 1H), 6.04-5.91 (m, 1H), 5.11-5.05 (m, 2H), 3.46 (dd, 2H).

### 2-[[5-[3-[4-(3-Allyl-2-fluoro-phenyl)-4,5,6,7-tetrahydro-1H-imidazo[4,5-c]pyridin-2-yl]-4-fluoro-phenoxy]-4,6-difluoro-indol-]-yl]methoxy]ethyl-trimethyl-silane

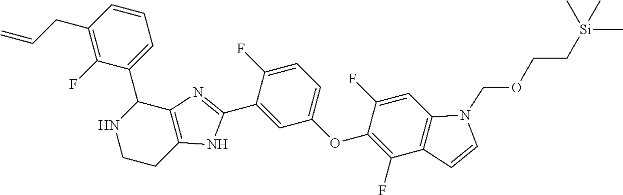 [SMILES: C=CCc1cccc(C2NCCc3[nH]c(-c4cc(Oc5c(F)cc6c(ccn6COCC[Si](C)(C)C)c5F)ccc4F)nc32)c1F]

Step C: To a stirred solution of 2-[2-[5-[4,6-difluoro-1-(2-trimethylsilylethoxymethyl)indol-5-yl]oxy-2-fluoro-phenyl]-1H-imidazol-5-yl]ethanamine (Step D, Example 11) (1.00 eq, 750 mg, 1.49 mmol) in ethanol (15 mL) was added 3-allyl-2-fluoro-benzaldehyde (1.20 eq, 295 mg, 1.80 mmol) followed by TEA (3.00 eq, 0.63 mL, 4.49 mmol). The mixture was heated at 70° C. for 24 h, cooled to room temperature, and concentrated. The residue was purified by flash chromatography on neutral alumina, eluting with 10-65% EtOAc in hexane, to give 2-[[5-[3-[4-(3-allyl-2-fluoro-phenyl)-4,5,6,7-tetrahydro-1H-imidazo[4,5-c]pyridin-2-yl]-4-fluoro-phenoxy]-4,6-difluoro-indol-1-yl]methoxy]ethyl-trimethyl-silane (504 mg, 0.777 mmol, 52% yield) as a pale-yellow, gummy solid. MS (ESI): m/z 649 [M+H]⁺.

### 2-[[5-[3-[4-(3-Allyl-2-fluoro-phenyl)-5-methyl-1,4,6,7-tetrahydroimidazo[4,5-c]pyridin-2-yl]-4-fluoro-phenoxy]-4,6-difluoro-indol-]-yl]methoxy]ethyl-trimethyl-silane

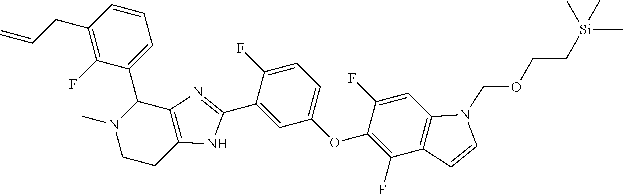 [SMILES: C=CCc1cccc(C2c3nc(-c4cc(Oc5c(F)cc6c(ccn6COCC[Si](C)(C)C)c5F)ccc4F)[nH]c3CCN2C)c1F]

Step D: To a stirred solution of 2-[[5-[3-[4-(3-allyl-2-fluoro-phenyl)-4,5,6,7-tetrahydro-1H-imidazo[4,5-c]pyridin-2-yl]-4-fluoro-phenoxy]-4,6-difluoro-indol-1-yl]methoxy]ethyl-trimethyl-silane (1.00 eq, 100 mg, 0.154 mmol) in methanol (2 mL) was added 38% (w/v) aqueous formaldehyde solution (0.50 mL, 19.0 mmol) followed by sodium cyanoborohydride (1.00 eq, 9.2 mg, 0.146 mmol). After stirring for 1 hour at rt, acetic acid (1.00 eq, 0.10 mL, 1.75 mmol) was added, and stirring was continued overnight. The reaction was quenched with water (10 mL). The pH was adjusted to ~9 with 6N aqueous sodium hydroxide and the mixture was extracted with EtOAc. The combined organic extracts were washed with water, brine, dried over sodium sulfate, and concentrated. The residue was purified by silica gel column chromatography, eluting with 20-80% EtOAc in hexane, to give 2-[[5-[3-[4-(3-allyl-2-fluoro-phenyl)-5-methyl-1,4,6,7-tetrahydroimidazo[4,5-c]pyridin-2-yl]-4-fluoro-phenoxy]-4,6-difluoro-indol-1-yl]methoxy]ethyl-trimethyl-silane (52 mg, 0.0785 mmol, 51% yield) as a pale-yellow solid. MS (ESI): m/z 663 [M+H]⁺.

### 3-[3-[2-[5-[4,6-Difluoro-1-(2-trimethylsilylethoxymethyl)indol-5-yl]oxy-2-fluoro-phenyl]-5-methyl-1,4,6,7-tetrahydroimidazo[4,5-c]pyridin-4-yl]-2-fluoro-phenyl]propane-1,2-diol

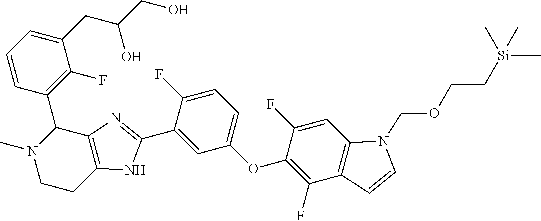

Step E: To a stirred solution of 2-[[5-[3-[4-(3-allyl-2-fluoro-phenyl)-5-methyl-1,4,6,7-tetrahydroimidazo[4,5-c]pyridin-2-yl]-4-fluoro-phenoxy]-4,6-difluoro-indol-1-yl]methoxy]ethyl-trimethyl-silane (1.00 eq, 150 mg, 0.226 mmol) in tert-butanol (7.5 mL) and water (7.5 mL) was added at room temperature potassium hexacyanoferrate(III) (3.00 eq, 224 mg, 0.679 mmol) followed by potassium carbonate (3.00 eq, 94 mg, 0.679 mmol) and Potassium osmate(VI) dihydrate (0.0100 eq, 0.83 mg, 0.00226 mmol). The reaction was stirred at room temperature for 24 h. Sodium sulfite was added to the reaction mixture and stirring continued for 2 hours. Excess tert-butanol was evaporated under reduced pressure and the residue was extracted with EtOAc. The organic extract was dried over sodium sulphate and concentrated. The residue was purified by silica gel column chromatography, eluting with 1-5% MeOH in DCM, to give 3-[3-[2-[5-[4,6-difluoro-1-(2-trimethylsilylethoxymethyl)indol-5-yl]oxy-2-fluoro-phenyl]-5-methyl-1,4,6,7-tetrahydroimidazo[4,5-c]pyridin-4-yl]-2-fluoro-phenyl]propane-1,2-diol (72 mg, 0.103 mmol, 46% yield) as an off-white solid. MS (ESI): m/z 697 [M+H]⁺.

### 3-[3-[2-[5-[(4,6-Difluoro-1H-indol-5-yl)oxy]-2-fluoro-phenyl]-5-methyl-1,4,6,7-tetrahydroimidazo[4,5-c]pyridin-4-yl]-2-fluoro-phenyl]propane-1,2-diol

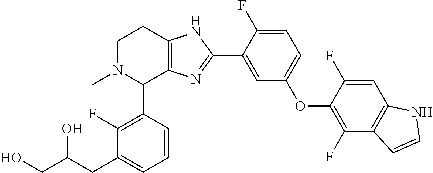 [SMILES: CN1CCc2[nH]c(-c3cc(Oc4c(F)cc5[nH]ccc5c4F)ccc3F)nc2C1c1cccc(CC(O)CO)c1F]

Step F: To a stirred solution of 3-[3-[2-[5-[4,6-difluoro-1-(2-trimethylsilylethoxymethyl)indol-5-yl]oxy-2-fluoro-phenyl]-5-methyl-1,4,6,7-tetrahydroimidazo[4,5-c]pyridin-4-yl]-2-fluoro-phenyl]propane-1,2-diol (1.00 eq, 30 mg, 0.0431 mmol) in THE (4 mL) was added at 0° C. tetrabutylammonium fluoride solution (1M in THF, 5.00 eq, 0.22 mL, 0.215 mmol). The mixture was maintained at room temperature for 30 min, refluxed with stirring for 5 h, and cooled to rt. The reaction mixture was diluted with water and extracted with EtOAc. The organic extract was washed with brine, dried over sodium sulphate, filtered, and concentrated. The residue was purified by silica gel preparative TLC, eluting with 10% MeOH in DCM, to give 3-[3-[2-[5-[(4,6-difluoro-1H-indol-5-yl)oxy]-2-fluoro-phenyl]-5-methyl-1,4,6,7-tetrahydroimidazo[4,5-c]pyridin-4-yl]-2-fluoro-phenyl]propane-1,2-diol (5.0 mg, 0.00849 mmol, 20% yield) as an off-white solid. MS (ESI): m/z 567 [M+H]⁺.

### Example 21. Synthesis of 3-[2-fluoro-3-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]phenyl]propane-1,2-diol

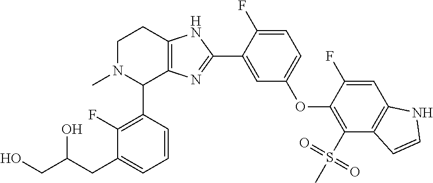 [SMILES: CN1CCc2[nH]c(-c3cc(Oc4c(F)cc5[nH]ccc5c4S(C)(=O)=O)ccc3F)nc2C1c1cccc(CC(O)CO)c1F]

### 3-(2,3-Dihydroxypropyl)-2-fluoro-benzaldehyde

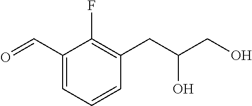 [SMILES: O=Cc1cccc(CC(O)CO)c1F]

Step A: To a stirred solution of 3-allyl-2-fluoro-benzaldehyde (1.00 eq, 250 mg, 1.52 mmol) in tert-butanol (4 mL) and water (4 mL) was added at room temperature potassium ferricyanide (3.00 eq, 1504 mg, 4.57 mmol) followed by K₂CO₃ (3.00 eq, 630 mg, 4.57 mmol) and potassium osmate (0.0100 eq, 5.6 mg, 0.0152 mmol). The reaction was stirred at room temperature for 24 h, diluted with ethyl acetate and filtered through a Celite pad. The filtrate was dried over sodium sulphate and concentrated under reduced pressure to give a crude product which was purified by preparative TLC (silica gel), eluting with 70% ethyl acetate in hexane, to give 3-(2,3-dihydroxypropyl)-2-fluoro-benzaldehyde (190 mg, 0.959 mmol, 63% yield) as a viscous syrup. ¹H NMR (300 MHz, CDCl₃) δ 10.37 (s, 1H), 7.77 (dt, 1H), 7.55 (dt, 1H), 7.23 (t, 1H), 4.07-3.99 (m, 1H), 3.75 (dd, 1H), 3.55 (m, 1H), 2.98-2.82 (m, 2H).

### 3-[2-Fluoro-3-[2-[2-fluoro-5-[6-fluoro-4-methylsulfonyl-]-(p-tolylsulfonyl)indol-5-yl]oxy-phenyl]-4,5,6,7-tetrahydro-1H-imidazo[4,5-c]pyridin-4-yl]phenyl]propane-1,2-diol

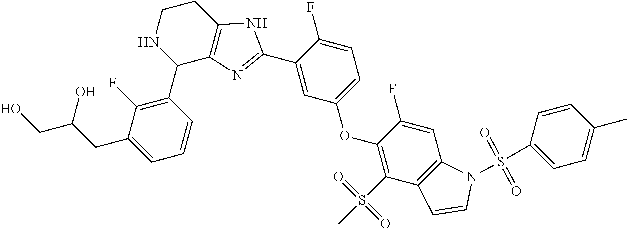 [SMILES: Cc1ccc(S(=O)(=O)n2ccc3c(S(C)(=O)=O)c(Oc4ccc(F)c(-c5nc6c([nH]5)CCNC6c5cccc(CC(O)CO)c5F)c4)c(F)cc32)cc1]

Step B: To flame dried 4 Å molecular sieves was added at RT a solution of 2-[2-[2-fluoro-5-[6-fluoro-4-methylsulfonyl-1-(p-tolylsulfonyl)indol-5-yl]oxy-phenyl]-1H-imidazol-4-yl]ethanamine (1.00 eq, 250 mg, 0.426 mmol) in ethanol (5 mL), followed by ethyl 3-(2,3-dihydroxypropyl)-2-fluoro-benzaldehyde (1.50 eq, 127 mg, 0.639 mmol) and TEA (3.00 eq, 129 mg, 1.28 mmol). The reaction mass was stirred at 70-80° C. for 16 h. The reaction mixture was cooled, filtered to remove the molecular sieves, and the volatiles removed under reduced pressure. The crude was purified by column chromatography on silica gel, eluted with 1-2% MeOH in DCM, to afford 3-[2-fluoro-3-[2-[2-fluoro-5-[6-fluoro-4-methylsulfonyl-1-(p-tolylsulfonyl)indol-5-yl]oxy-phenyl]-4,5,6,7-tetrahydro-1H-imidazo[4,5-c]pyridin-4-yl]phenyl]propane-1,2-diol (135 mg, 0.176 mmol, 41% yield) as a pale yellow solid. MS (ESI): m/z 767 [M+H]⁺.

### 3-(2-fluoro-3-(2-(2-fluoro-5-((6-fluoro-4-(methylsulfonyl)-1-tosyl-1H-indol-5-yl)oxy)phenyl)-5-methyl-4,5,6,7-tetrahydro-1H-imidazo[4,5-c]pyridin-4-yl)phenyl)propane-1,2-diol

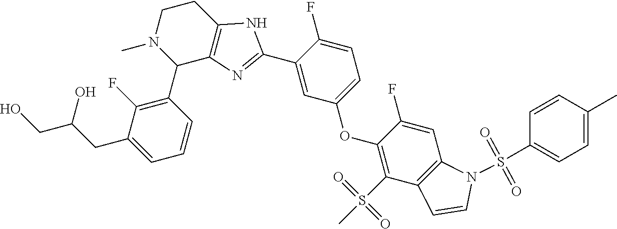 [SMILES: Cc1ccc(S(=O)(=O)n2ccc3c(S(C)(=O)=O)c(Oc4ccc(F)c(-c5nc6c([nH]5)CCN(C)C6c5cccc(CC(O)CO)c5F)c4)c(F)cc32)cc1]

Step C: To a stirred solution of 3-[2-fluoro-3-[2-[2-fluoro-5-[6-fluoro-4-methylsulfonyl-1-(p-tolylsulfonyl)indol-5-yl]oxy-phenyl]-4,5,6,7-tetrahydro-1H-imidazo[4,5-c]pyridin-4-yl]phenyl]propane-1,2-diol (1.00 eq, 30 mg, 0.0391 mmol) in methanol (3 mL) were added formaldehyde (3.00 eq, 3.5 mg, 0.117 mmol) and platinum(IV) oxide hydrate (0.100 eq, 0.89 mg, 0.00391 mmol). The mixture was stirred for 16 h under a hydrogen atmosphere at ambient temperature. The reaction was filtered through a Celite pad, the pad was washed with ethanol, and the combined filtrates were concentrated under reduced pressure. The residue was purified by flash chromatography on silica gel, eluting with 2-4% MeOH in DCM, to afford 3-[2-fluoro-3-[2-[2-fluoro-5-[6-fluoro-4-methylsulfonyl-1-(p-tolylsulfonyl)indol-5-yl]oxy-phenyl]-5-methyl-1,4,6,7-tetrahydroimidazo[4,5-c]pyridin-4-yl]phenyl]propane-1,2-diol (90 mg). MS (ESI): m/z 781 [M+H]⁺.

### 3-[2-Fluoro-3-[2-[2-fluoro-5-[(6-fluoro-4-methylsulfonyl-1H-indol-5-yl)ox]phenyl]-5-methyl-1,4,6,7-tetrahydroimidazo[4,5-c]pyridin-4-yl]phenyl]propane-],2-diol

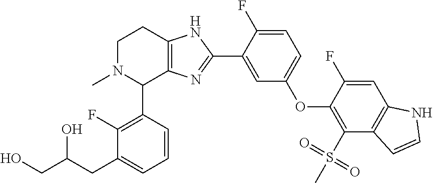 [SMILES: CN1CCc2[nH]c(-c3cc(Oc4c(F)cc5[nH]ccc5c4S(C)(=O)=O)ccc3F)nc2C1c1cccc(CC(O)CO)c1F]

Step D: To a stirred solution of 3-[2-fluoro-3-[2-[2-fluoro-5-[6-fluoro-4-methylsulfonyl-1-(p-tolylsulfonyl)indol-5-yl]oxy-phenyl]-5-methyl-1,4,6,7-tetrahydroimidazo[4,5-c]pyridin-4-yl]phenyl]propane-1,2-diol (1.00 eq, 90 mg, 0.115 mmol) in methanol (3 mL) was added 1N aqueous NaOH (1.50 eq.) and stirred at RT for 16 h. The reaction mixture was filtered through a Celite pad, the pad was washed with ethanol. The combined filtrates were concentrated under reduced pressure. The residue was purified by preparative TLC on silica gel, eluting with 5% MeOH in DCM. The partially purified desired product was triturated with pentane to give pure 3-[2-fluoro-3-[2-[2-fluoro-5-[(6-fluoro-4-methylsulfonyl-1H-indol-5-yl)oxy]phenyl]-5-methyl-1,4,6,7-tetrahydroimidazo[4,5-c]pyridin-4-yl]phenyl]propane-1,2-diol (4.0 mg, 0.00638 mmol, 6% yield) as a colorless semi-solid. MS (ESI): m/z 627 [M+H]⁺.

### Example 22. Synthesis of 3-[2-fluoro-3-[2-[2-fluoro-5-[(6-fluoro-4-methylsulfonyl-1H-indol-5-yl)oxy]phenyl]-3,4,5,6,7,8-hexahydroimidazo[4,5-c]azepin-4-yl]phenyl]propanoic acid

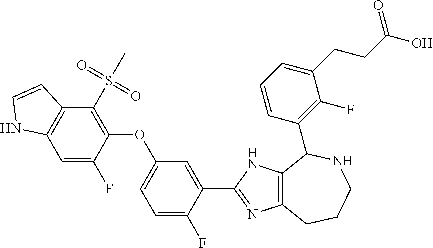 [SMILES: CS(=O)(=O)c1c(Oc2ccc(F)c(-c3nc4c([nH]3)C(c3cccc(CCC(=O)O)c3F)NCCC4)c2)c(F)cc2[nH]ccc12]

### 2-[3-[2-[2-Fluoro-5-[6-fluoro-4-methylsulfonyl-1-(p-tolylsulfonyl)indol-5-yl]oxy-phenyl]-1H-imidazol-4-yl]propyl]isoindoline-1,3-dione

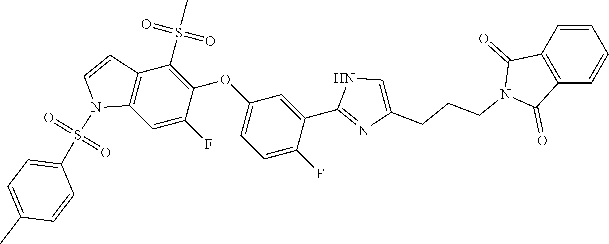 [SMILES: Cc1ccc(S(=O)(=O)n2ccc3c(S(C)(=O)=O)c(Oc4ccc(F)c(-c5nc(CCCN6C(=O)c7ccccc7C6=O)c[nH]5)c4)c(F)cc32)cc1]

Step A: To a stirred solution of 2-(5-chloro-4-oxo-pentyl)isoindoline-1,3-dione (1.20 eq, 736 mg, 2.77 mmol) and 2-fluoro-5-[6-fluoro-4-methylsulfonyl-1-(p-tolylsulfonyl)indol-5-yl]oxy-benzamidine (Step C, Example 7) (1.00 eq, 1.20 g, 2.31 mmol) in DMF (15 mL) was added sodium bicarbonate (3.00 eq, 582 mg, 6.93 mmol), and stirring continued at rt for 30 minutes. The reaction mixture was then stirred for 16 h at 70° C. After cooling to rt, ice-cold water (100 mL) was added. The mixture was extracted with EtOAc (3×25 mL), the combined organic extracts were washed with brine, dried over anhydrous sodium sulphate, and concentrated. The residue was purified by flash chromatography on silica gel to afford 2-[3-[2-[2-fluoro-5-[6-fluoro-4-methylsulfonyl-1-(p-tolylsulfonyl)indol-5-yl]oxy-phenyl]-1H-imidazol-4-yl]propyl]isoindoline-1,3-dione (1.20 g, 1.46 mmol, 63% yield) as an off-white solid. MS (ESI): m/z 731 [M+H]⁺.

### 3-[2-[2-Fluoro-5-[6-fluoro-4-methylsulfonyl-]-(p-tolylsulfonyl)indol-5-yl]oxy-phenyl]-1H-imidazol-5-yl]propan-1-amine

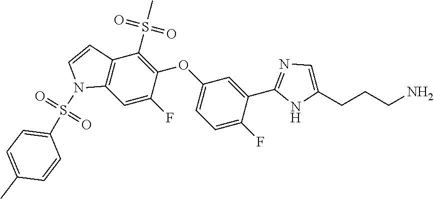 [SMILES: Cc1ccc(S(=O)(=O)n2ccc3c(S(C)(=O)=O)c(Oc4ccc(F)c(-c5ncc(CCCN)[nH]5)c4)c(F)cc32)cc1]

Step B: To a stirred solution of 2-[3-[2-[2-fluoro-5-[6-fluoro-4-methylsulfonyl-1-(p-tolylsulfonyl)indol-5-yl]oxy-phenyl]-1H-imidazol-5-yl]propyl]isoindoline-1,3-dione (1.00 eq, 1.00 g, 1.22 mmol) in ethanol (20 mL) was added hydrazine hydrate (3.00 eq, 0.18 g, 3.65 mmol) at RT. The reaction mass was stirred for 16 h at 50° C., cooled to RT, diluted with MTBE (15 mL), and stirred for another 20 minutes. The solids were filtered off and rinsed with MTBE (15 mL). The combined filtrates were concentrated under reduced pressure to afford 3-[2-[2-fluoro-5-[6-fluoro-4-methylsulfonyl-1-(p-tolylsulfonyl)indol-5-yl]oxy-phenyl]-1H-imidazol-5-yl]propan-1-amine (0.61 g, 0.863 mmol, 71% yield) as a colorless syrup. MS (ESI): m/z 601 [M+H]⁺.

### Ethyl (E)-3-[2-fluoro-3-[2-[2-fluoro-5-[6-fluoro-4-methylsulfonyl-1-(p-tolylsulfonyl)indol-5-yl]oxy-phenyl]-3,4,5,6,7,8-hexahydroimidazo[4,5-c]azepin-4-yl]phenyl]prop-2-enoate

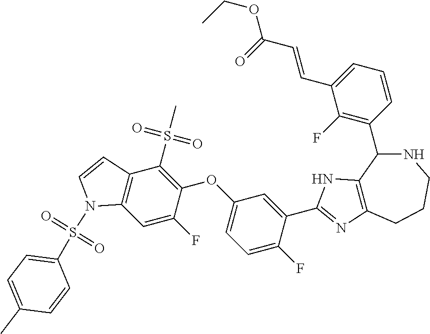 [SMILES: CCOC(=O)/C=C/c1cccc(C2NCCCc3nc(-c4cc(Oc5c(F)cc6c(ccn6S(=O)(=O)c6ccc(C)cc6)c5S(C)(=O)=O)ccc4F)[nH]c32)c1F]

Step C: To a stirred solution of 3-[2-[2-fluoro-5-[6-fluoro-4-methylsulfonyl-1-(p-tolylsulfonyl)indol-5-yl]oxy-phenyl]-1H-imidazol-4-yl]propan-1-amine (1.00 eq, 550 mg, 0.916 mmol) in ethanol (3 mL) was added at RT ethyl (E)-3-(2-fluoro-3-formyl-phenyl)prop-2-enoate (1.20 eq, 245 mg, 1.10 mmol) and TEA (1.50 eq, 0.19 mL, 1.37 mmol) at RT. The reaction mixture was stirred at 70-80° C. for r 12 days. The reaction mass was cooled to RT, quenched with water (10 mL) and extracted with EtOAc (2×10 mL). The combined organic extracts were washed with brine (10 mL), dried over anhydrous Na₂SO₄, filtered, and concentrated under reduced pressure. The crude was purified by flash column chromatography on silica gel, eluting with 50-100% EtOAc in hexanes, to give ethyl (E)-3-[2-fluoro-3-[2-[2-fluoro-5-[6-fluoro-4-methylsulfonyl-1-(p-tolylsulfonyl)indol-5-yl]oxy-phenyl]-3,4,5,6,7,8-hexahydroimidazo[4,5-c]azepin-4-yl]phenyl]prop-2-enoate (305 mg, 0.309 mmol, 33% yield) as a brown solid. MS (ESI): m/z 805 [M+H]⁺.

### Ethyl 3-[2-fluoro-3-[2-[2-fluoro-5-[6-fluoro-4-methylsulfonyl-1-(p-tolylsulfonyl)indol-5-yl]oxy-phenyl]-3,4,5,6,7,8-hexahydroimidazo[4,5-c]azepin-4-yl]phenyl]propanoate

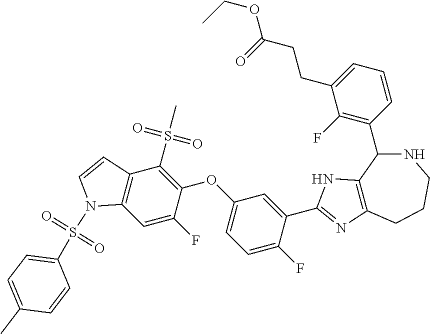 [SMILES: CCOC(=O)CCc1cccc(C2NCCCc3nc(-c4cc(Oc5c(F)cc6c(ccn6S(=O)(=O)c6ccc(C)cc6)c5S(C)(=O)=O)ccc4F)[nH]c32)c1F]

Step D: To a stirred solution of ethyl (E)-3-[2-fluoro-3-(3,4,5,6,7,8-hexahydroimidazo[4,5-c]azepin-4-yl)phenyl]prop-2-enoate;6-fluoro-5-(4-fluoro-3-methyl-phenoxy)₀₋₄-methylsulfonyl-1-(p-tolylsulfonyl)indole (1.00 eq, 130 mg, 0.158 mmol) in methanol (5 mL) and ethyl acetate (7.5 mL) was added nickel(II) acetate tetrahydrate (1.50 eq, 59 mg, 0.238 mmol). The reaction mixture was chilled (0° C.) and sodium borohydride (3.00 eq, 18 mg, 0.475 mmol) was added portion wise. The reaction mixture was stirred at room temperature under a H₂ atmosphere for 2 h, diluted with water (50 mL), and extracted with EtOAc (100 mL). The separated organic layer was washed with brine (20 mL), dried over anhydrous Na₂SO₄, filtered and concentrated under reduced pressure to afford ethyl 3-[2-fluoro-3-[2-[2-fluoro-5-[6-fluoro-4-methylsulfonyl-1-(p-tolylsulfonyl)indol-5-yl]oxy-phenyl]-3,4,5,6,7,8-hexahydroimidazo[4,5-c]azepin-4-yl]phenyl]propanoate (95 mg, 0.118 mmol, 74% yield) as a pale-brown solid. MS (ESI): m/z 807 [M+H]⁺.

### 3-[2-Fluoro-3-[2-[2-fluoro-5-[(6-fluoro-4-methylsulfonyl-1H-indol-5-yl)oxy]phenyl]-3,4,5,6,7,8-hexahydroimidazo[4,5-c]azepin-4-yl]phenyl]propanoic acid

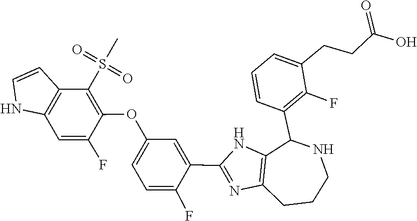 [SMILES: CS(=O)(=O)c1c(Oc2ccc(F)c(-c3nc4c([nH]3)C(c3cccc(CCC(=O)O)c3F)NCCC4)c2)c(F)cc2[nH]ccc12]

Step E: To a stirred and chilled (0° C.) solution of ethyl 3-[2-fluoro-3-[2-[2-fluoro-5-[6-fluoro-4-methylsulfonyl-1-(p-tolylsulfonyl)indol-5-yl]oxy-phenyl]-3,4,5,6,7,8-hexahydroimidazo[4,5-c]azepin-4-yl]phenyl]propanoate (1.00 eq, 95 mg, 0.118 mmol) in methanol (5 mL) was added 6N aqueous NaOH (76.4 eq, 1.5 mL, 9.00 mmol). The mixture was stirred at room temperature for 20 h and concentrated under reduced pressure. The residue was purified by preparative TLC (silica gel), eluting with 20% of methanol in DCM, to obtained the desired 3-[2-fluoro-3-[2-[2-fluoro-5-[(6-fluoro-4-methylsulfonyl-1H-indol-5-yl)oxy]phenyl]-3,4,5,6,7,8-hexahydroimidazo[4,5-c]azepin-4-yl]phenyl]propanoic acid (2.8 mg, 0.00418 mmol, 4% yield) as a pale-yellow solid. MS (ESI): m/z 625 [M+H]⁺.

### Biological Assays

### Example 23. Aggregation Analysis Using Differential Static Light Scattering (DSLS)

Purified recombinant NBD1 was produced using previously described methods (A. Schmidt, J. L. Mendoza, P. J. Thomas (2011) Biochemical and Biophysical Approaches to Probe CFTR Structure (365-376) M. D. Amaral, K. Kunzelmann (eds.), Cystic Fibrosis, Methods in Molecular Biology 741, Springer Science+Business Media). The effect of test compounds on thermal stability of NBD1 was evaluated by differential static light scattering (DSLS) using the Harbinger Stargazer-384 instrument (Epiphyte Three, Toronto, Canada). Test compounds were dissolved and diluted to desired concentrations in 100% DMSO. The compounds or DMSO controls (100 nL) were stamped into wells of a 385-well low volume optical plate (Corning Inc., Corning, NY) using the Echo 555 acoustic liquid handler (Labcyte Inc., San Jose, CA).

NBD1 protein was diluted to 0.2 mg/ml in S200 buffer (50 mM Tris-HCl, 150 mM NaCl, 5 mM MgCl₂, 2 mM ATP, 2 mM DTT, pH7.6) containing 1% glycerol. 10 μL of protein solution was aliquoted into the 384-well plate harboring the test compounds and 10 μL mineral oil was overlayed onto the protein solution, using the epMotion robotic liquid handler (Eppendorf North America, Hauppauge, NY). After placing into the Stargazer instrument, the plate was heated at 1° C. per minute to 70° C. Images were captured from 25° C. to 70° C. every 0.5° C. At the end of the experiment run, instrument software integrated image files and analyzed data automatically. A linear regression curve was generated for each well, representing the increase in light scattering over time. A temperature of aggregation (Tagg) was calculated based on the inflection point of the curve. To better compare data across experiments the average Tagg for DMSO control wells was calculated and subtracted from values for wells containing compounds to obtain a “ΔTagg” value. These ΔTagg values reflect stabilizing efficacy of the compounds.

Data for Compounds 1-6 are provided in Table 2 below.

TABLE 2

| | DSLS NBD1/3D |
|---|---|
| | Human Stability |
| | delta-T (° C.) |
| Example No | @ 100 (μM) |
| Example 1 | B |
| Example 2 | B |
| Example 3 | A |
| Example 4 | C |
| Example 5 | B |
| Example 6 | A |
| Example 7 | A |
| Example 8 | A |
| Example 9 | B |
| Example 10 | C |
| Example 11 | A |
| Example 12 | A |
| Example 13 | B |
| Example 14 | A |
| Example 15 | C |
| Example 16 | A |
| Example 17 | B |
| Example 18 | B |
| Example 19 | A |
| Example 20 | B |
| Example 21 | A |
| Example 22 | A |

*A: T ≥ 8° C.; B: 4-8° C.; C: ≤4° C.

### Example 24. TECC24 AUC Fold Over DMSO @10 μM

The effects of a test agent on CFTR-mediated transepithelial chloride transport was measured using TECC24 recording analysis. Test agents were solubilized in DMSO. Solubilized test agents were mixed with incubation medium containing DMEM/F12, Ultroser G (2%; Crescent Chemical, catalog #67042), Hyclone Fetal Clone 11 (2%; GE Healthcare, catalog #SH30066.02), bovine brain extract (0.25%; Lonza, catalog #CC-4098), insulin (2.5 μg/mL), IL-13 (10 ng/mL), hydrocortisone (20 nM), transferrin (2.5 g/mL), triiodothyronine (500 nM), ethanolamine (250 nM), epinephrine (1.5 μM), phosphoethanolamine (250 nM), and retinoic acid (10 nM). Primary human bronchial epithelial cells from a ΔF508 homozygous CF donor (CF-HBE cells; from University of North Carolina Cystic Fibrosis Tissue Procurement Center), grown on Transwell HTS 24-well cell culture inserts (Costar, catalog #3378), were exposed to test agents or controls dissolved in incubation medium. The CF-HBE cells were cultured at 36.5° C. for 48 hours before TECC24 recordings were performed in the presence or absence of test agent, a positive control or vehicle (DMSO).

Following incubation, the transwell cell culture inserts containing the test agent or control-treated CF-HBE cells were loaded onto a TECC24 apparatus (TECC v7 or MTECC v2; EP Design) to record the transepithelial voltage (VT) and resistance (TEER) using 4 AgCl electrodes per well configured in current-clamp mode. The apical and basolateral bath solutions both contained (in mM) 140 NaCl, 5 KCl, 2 CaCl₂), 1 MgCl₂, 10 Hepes, and 10 glucose (adjusted to pH 7.4 with NaOH). To inhibit basal Na+ absorption, the ENaC inhibitor benzamil (10 μM) was added to the bath. Then, the adenylate cyclase activator, forskolin (10 μM), was added to the bath to activate CFTR. The forskolin-stimulated Cl− transport was halted by addition of CFTR inhibitor-172 (20 μM) to the bath at the end of the experiment to confirm specificity. VT and TEER recordings were digitally acquired at routine intervals using TECC or MTECC software (EP Design). VT and TEER were transformed into equivalent transepithelial Cl− current (IEQ), and the Area Under the Curve (AUC) of the IEQ time course between forskolin and CFTR inhibitor-172 addition is generated using Excel (Microsoft). Efficacy is expressed as the ratio of the test agent AUC divided by vehicle AUC. EC50s based on AUC are generated using the non-linear regression log(agonist) vs. response function in Prism software (Graphpad) with HillSlope fixed=1.

If a test agent increased the AUC of the forskolin-stimulated IEQ relative to vehicle in CF-HBE cells, and this increase was inhibited by CFTR inhibitor-172, then the test agent was considered a CFTR corrector. The data is shown in Table 3 below.

TABLE 3

| Example | TECC24 @ 10 |
|---|---|
| No. | uM over DMSO |
| Example 1 | C |
| Example 3 | A |
| Example 6 | A |
| Example 6 | C |
| Example 8 | C |
| Example 11 | C |
| Example 12 | B |
| Example 22 | C |

ND = Not determined; “A” refers to AUC @10 uM >=12; “B” refers to AUC @10 uM between 4-12; “C” refers to AUC @10 uM <= 4.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 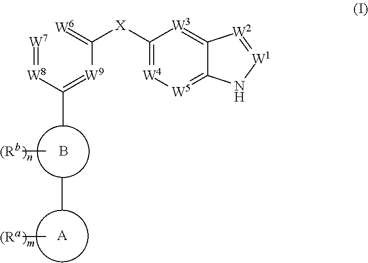
or a pharmaceutically acceptable salt thereof wherein
W¹ is selected from the group consisting of —C(H)═, and —N═;
W² is selected from the group consisting of —C(H)═, —C(R^{d})═, and —N═;
W³ is selected from the group consisting of —C(H)═, —C(R^{d})═, and —N═;
W⁴ is selected from the group consisting of —C(H)═, —C(R^{d4})═, and —N═;
W⁵ is selected from the group consisting of —C(H)═, —C(R^{d5})═, and —N═;
W⁶ is selected from the group consisting of —C(H)═, —C(R^{∘})═, and —N═;
W⁷ is selected from the group consisting of —C(H)═, —C(R^{∘})═, and —N═;
W⁸ is selected from the group consisting of —C(H)═, —C(R^{∘})═, and —N═;
W⁹ is selected from the group consisting of —C(H)═, —C(R⁹)═, and —N═;
Ring A is optionally substituted phenyl or 5-10 membered heteroaryl;
Ring B is an optionally substituted 8-10 membered fused heteroaryl;
each R^{a} is independently selected from the group consisting of halogen, oxo —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl,
wherein each R^{a} is independently substituted with 0-4 instances of R^{aa},
each R^{aa} is independently selected from the group consisting of halogen, oxo, —COOH, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, —SO(NR²)R¹, —SO₂N(R²)R¹, and —N(H)C(O)N(R¹)₂,
wherein two instances of R^{aa} are optionally taken together with any intervening atoms to form an optionally substituted 5-6 membered heterocyclyl ring;
each R^{b} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl,
wherein two instances of R^{b} are optionally taken together with any intervening atoms to form an optionally substituted 5-6 membered carbocyclyl or optionally substituted 5-6 membered heterocyclyl ring;
each R^{c} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{d} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl; R^{c9} is halogen;
R^{d4} is halogen;
R^{d5} is halogen;
each R¹ is independently selected from the group consisting of hydrogen, —(CH₂)₁₋₃R², —C(O)R², —(CH₂)₁₋₃OR², optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
wherein two instances of R¹ are optionally taken together with any intervening atoms to form an optionally substituted 3-7 membered heterocyclyl ring;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
X is selected from the group consisting of —O—, —S—, —S(O)—, —S(O)₂—; —SO(NR²)—, —C(R¹)(R²)—, and —C(O)—;
n is 0, 1, 2, or 3; and
m is 0, 1, 2, or 3.

2. The compound of claim 1, wherein W¹ is —N═.

3. The compound of claim 1, wherein W² is —N═.

4. The compound of claim 1, wherein W³ is —N═.

5. The compound of claim 1, wherein W⁴ is —N═.

6. The compound of claim 1, wherein when W⁸ is —N═, W¹, W², W³, and W⁴ are each not —N═.

7. The compound of claim 1, wherein the compound is of Formula (I-a), (I-b), (I-c), (I-d), (I-e), or (I-f): 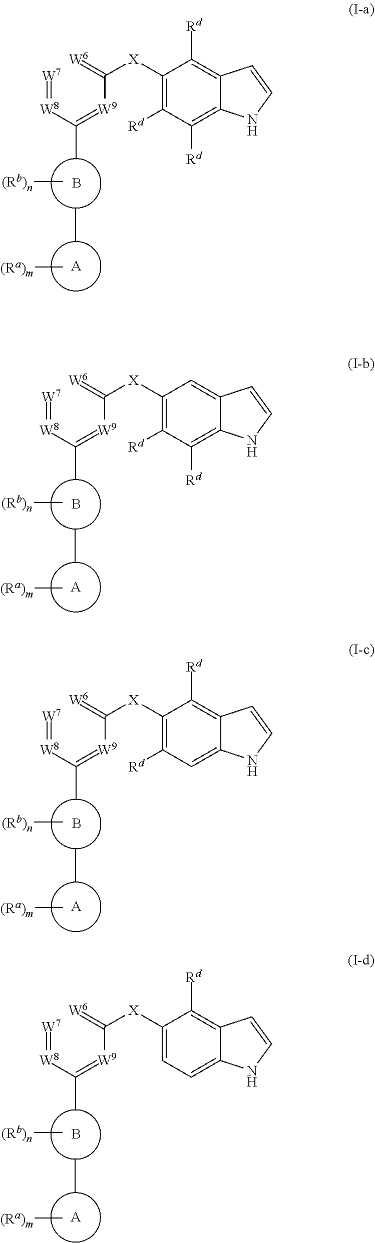 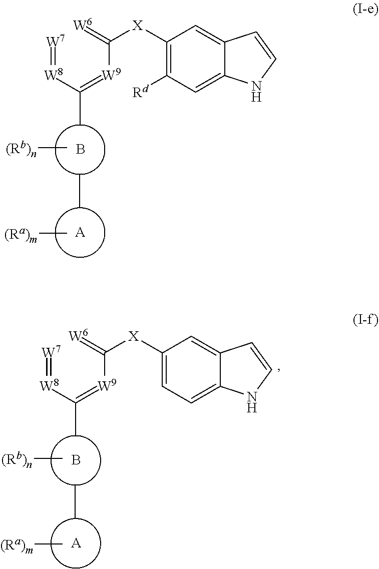
or a pharmaceutically acceptable salt thereof.

8. The compound of any of claims 1-7, wherein
W⁶ is —C(H)═;
W⁷ is —C(H)═;
W⁸ is —C(H)═; and
W⁹ is —N═.

9. The compound of any of claims 1-7, wherein
W⁶ is —C(H)═;
W⁷ is —C(H)═;
W⁸ is —N═; and
W⁹ is —C(H)═.

10. The compound of any of claims 1-7, wherein
W⁶ is —C(H)═;
W⁷ is —C(H)═;
W⁸ is —C(H)═; and
W⁹ is —C(H)═.

11. The compound of any of claims 1-7, wherein
W⁶ is —C(R^{c})═;
W⁷ is —C(H)═;
W⁸ is —C(H)═; and
W⁹ is —C(H)═.

12. The compound of any of claims 1-7, wherein
W⁶ is —C(H)═;
W⁷ is —C(H)═;
W⁸ is —C(R^{∘})═; and
W⁹ is —C(H)═.

13. The compound of any of claims 1-12, wherein X is —O—.

14. The compound of any of claims 1-13, wherein Ring A is optionally substituted phenyl.

15. The compound of any of claims 1-13, wherein Ring A is optionally substituted pyridinyl.

16. The compound of any of claims 1-13, wherein Ring A is selected from the group consisting of thiophenyl, pyrazolyl, pyrrolyl, and thiazolyl.

17. The compound of claim 14, wherein Ring A is selected from the group consisting of

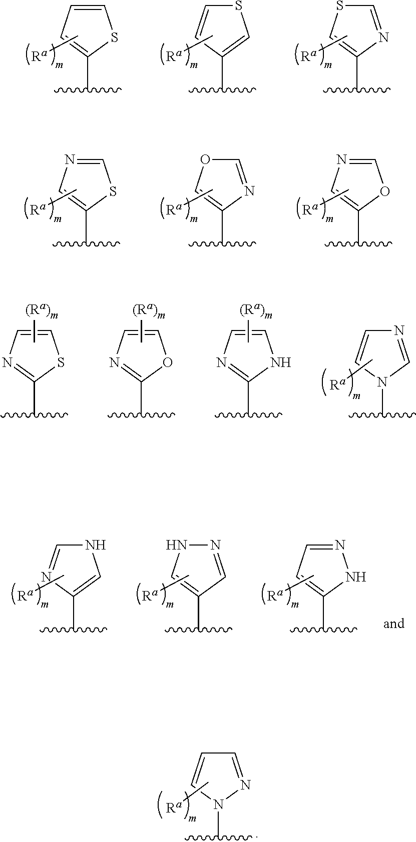

18. The compound of claim 14, wherein Ring A is selected from the group consisting of

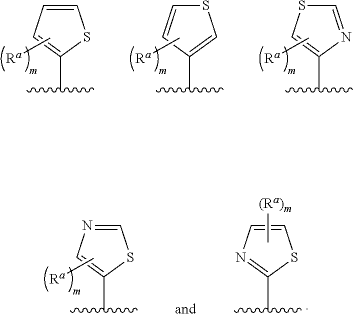

19. The compound of any of claims 1-18, wherein Ring B is selected from the group consisting of 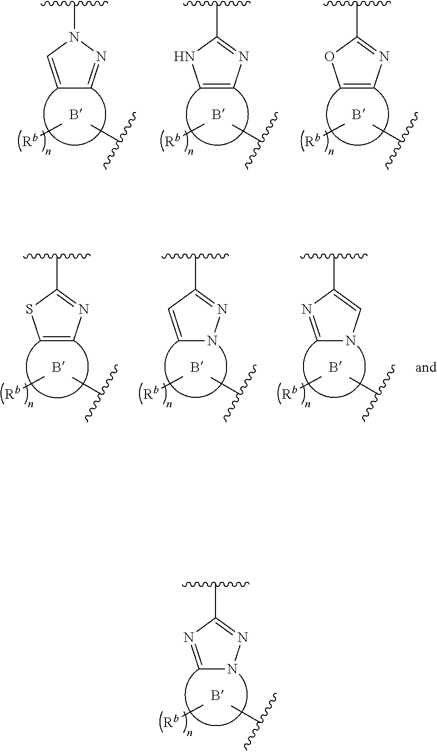
wherein B′ is selected from the group consisting of optionally substituted 5-7 carbocyclyl, optionally substituted 5-7 heterocyclyl, and optionally substituted 5-7 heteroaryl.

20. The compound of any of claims 1-18, wherein Ring B is selected from the group consisting of

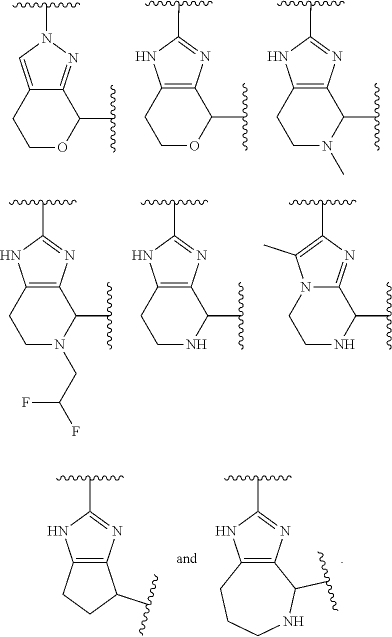

21. The compound of any of claims 1-14, wherein each R^{a} is independently selected from halogen or optionally substituted C₁-C₆ alkyl, wherein each R^{a} is independently substituted with 0-4 instances of R^{aa}.

22. The compound of claim 21, wherein each R^{a} is independently selected from the group consisting of fluoro, —CH₂CH₂COOH, —CH₂CH(Me)CO₂H, and —CH₂CH(OH)CH₂(OH).

23. The compound of any of claims 1-22, wherein R^{d} is halogen.

24. The compound of claim 23, wherein R^{d} is fluoro.

25. The compound of any of claims 1-24, wherein R^{d4} is halogen.

26. The compound of claim 25, wherein R^{d4} is fluoro.

27. The compound of any of claims 1-26, wherein R^{d5} is halogen.

28. The compound of claim 27, wherein R^{d}₅ is fluoro.

29. A compound selected from the group consisting of

| Compound No. | Structure |
|---|---|
| 1 | 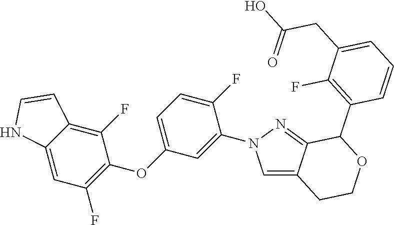 [SMILES: O=C(O)Cc1cccc(C2OCCc3cn(-c4cc(Oc5c(F)cc6[nH]ccc6c5F)ccc4F)nc32)c1F] |
| 2 | 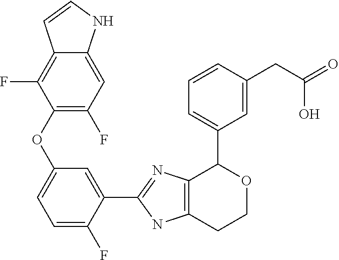 [SMILES: O=C(O)Cc1cccc(C2OCCc3[nH]c(-c4cc(Oc5c(F)cc6[nH]ccc6c5F)ccc4F)nc32)c1] |
| 3 | 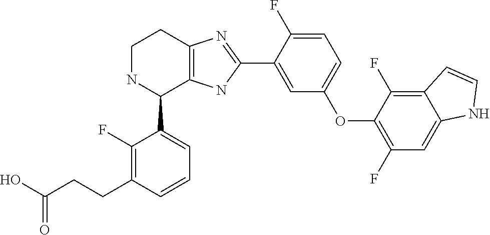 [SMILES: O=C(O)CCc1cccc([C@H]2NCCc3nc(-c4cc(Oc5c(F)cc6[nH]ccc6c5F)ccc4F)[nH]c32)c1F] |
| 4 | 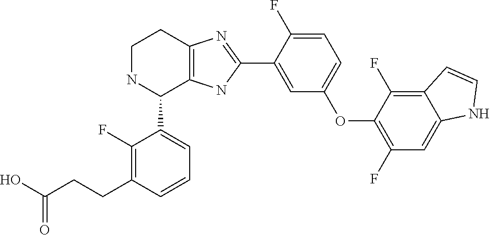 [SMILES: O=C(O)CCc1cccc([C@@H]2NCCc3nc(-c4cc(Oc5c(F)cc6[nH]ccc6c5F)ccc4F)[nH]c32)c1F] |
| 5 | 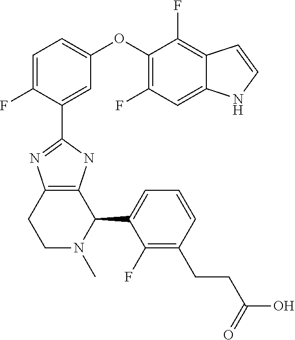 [SMILES: CN1CCc2nc(-c3cc(Oc4c(F)cc5[nH]ccc5c4F)ccc3F)[nH]c2[C@H]1c1cccc(CCC(=O)O)c1F] |
| 6 | 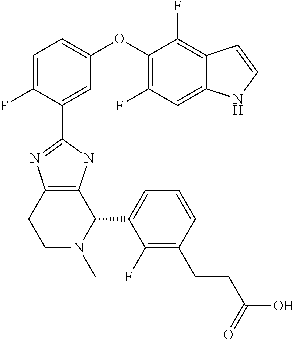 [SMILES: CN1CCc2nc(-c3cc(Oc4c(F)cc5[nH]ccc5c4F)ccc3F)[nH]c2[C@@H]1c1cccc(CCC(=O)O)c1F] |
| 7 | 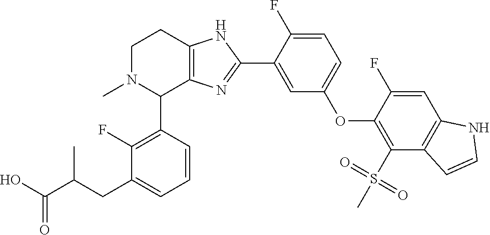 [SMILES: CC(Cc1cccc(C2c3nc(-c4cc(Oc5c(F)cc6[nH]ccc6c5S(C)(=O)=O)ccc4F)[nH]c3CCN2C)c1F)C(=O)O] |
| 8 | 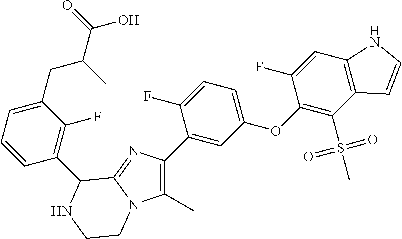 [SMILES: Cc1c(-c2cc(Oc3c(F)cc4[nH]ccc4c3S(C)(=O)=O)ccc2F)nc2n1CCNC2c1cccc(CC(C)C(=O)O)c1F] |
| 9 | 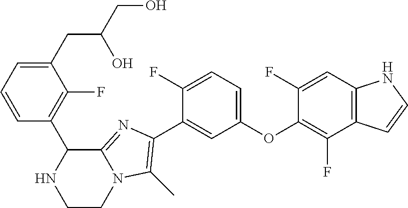 [SMILES: Cc1c(-c2cc(Oc3c(F)cc4[nH]ccc4c3F)ccc2F)nc2n1CCNC2c1cccc(CC(O)CO)c1F] |
| 10 | 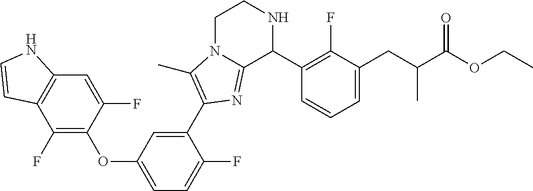 [SMILES: CCOC(=O)C(C)Cc1cccc(C2NCCn3c2nc(-c2cc(Oc4c(F)cc5[nH]ccc5c4F)ccc2F)c3C)c1F] |
| 11 | 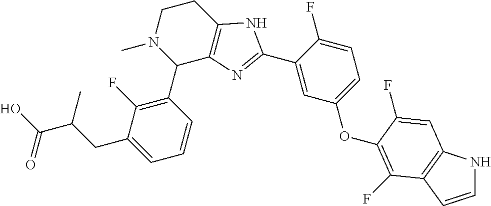 [SMILES: CC(Cc1cccc(C2c3nc(-c4cc(Oc5c(F)cc6[nH]ccc6c5F)ccc4F)[nH]c3CCN2C)c1F)C(=O)O] |
| 12 | 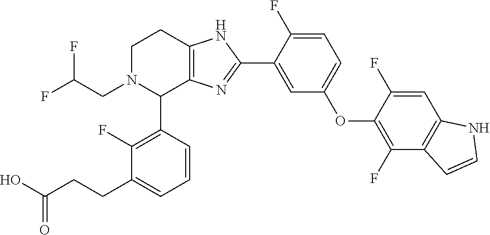 [SMILES: O=C(O)CCc1cccc(C2c3nc(-c4cc(Oc5c(F)cc6[nH]ccc6c5F)ccc4F)[nH]c3CCN2CC(F)F)c1F] |
| 13 | 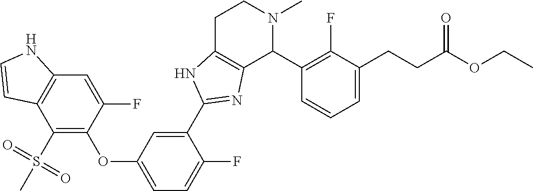 [SMILES: CCOC(=O)CCc1cccc(C2c3nc(-c4cc(Oc5c(F)cc6[nH]ccc6c5S(C)(=O)=O)ccc4F)[nH]c3CCN2C)c1F] |
| 14 | 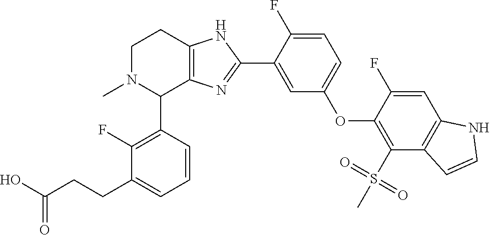 [SMILES: CN1CCc2[nH]c(-c3cc(Oc4c(F)cc5[nH]ccc5c4S(C)(=O)=O)ccc3F)nc2C1c1cccc(CCC(=O)O)c1F] |
| 15 | 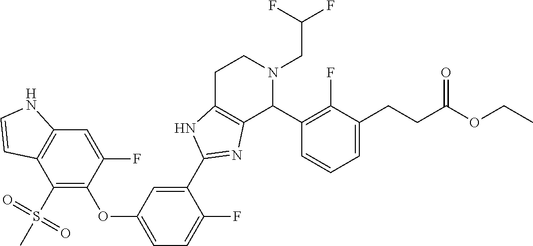 [SMILES: CCOC(=O)CCc1cccc(C2c3nc(-c4cc(Oc5c(F)cc6[nH]ccc6c5S(C)(=O)=O)ccc4F)[nH]c3CCN2CC(F)F)c1F] |
| 16 | 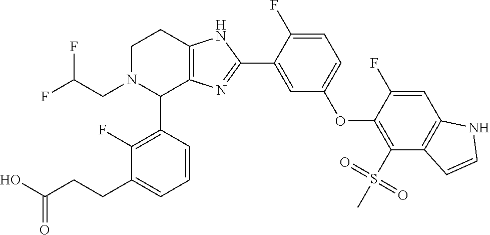 [SMILES: CS(=O)(=O)c1c(Oc2ccc(F)c(-c3nc4c([nH]3)CCN(CC(F)F)C4c3cccc(CCC(=O)O)c3F)c2)c(F)cc2[nH]ccc12] |
| 17 | 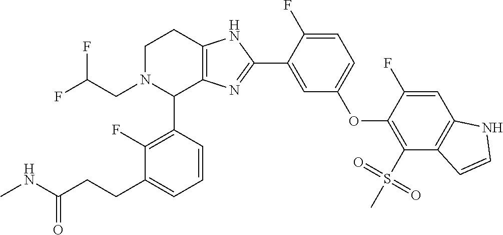 [SMILES: CNC(=O)CCc1cccc(C2c3nc(-c4cc(Oc5c(F)cc6[nH]ccc6c5S(C)(=O)=O)ccc4F)[nH]c3CCN2CC(F)F)c1F] |
| 18 | 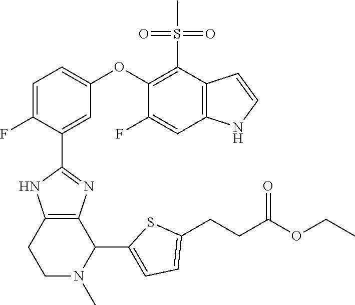 [SMILES: CCOC(=O)CCc1ccc(C2c3nc(-c4cc(Oc5c(F)cc6[nH]ccc6c5S(C)(=O)=O)ccc4F)[nH]c3CCN2C)s1] |
| 19 | 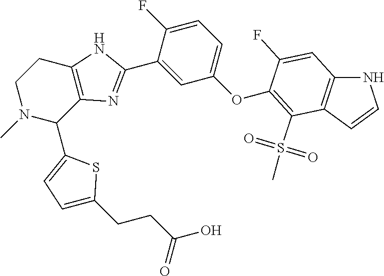 [SMILES: CN1CCc2[nH]c(-c3cc(Oc4c(F)cc5[nH]ccc5c4S(C)(=O)=O)ccc3F)nc2C1c1ccc(CCC(=O)O)s1] |
| 20 | 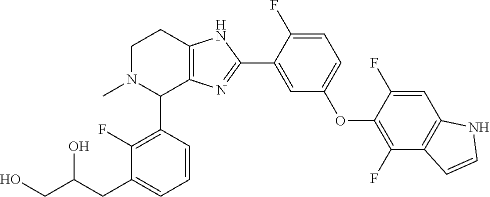 [SMILES: CN1CCc2[nH]c(-c3cc(Oc4c(F)cc5[nH]ccc5c4F)ccc3F)nc2C1c1cccc(CC(O)CO)c1F] |
| 21 | 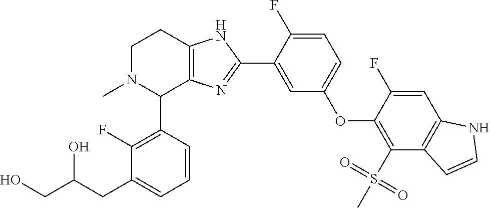 [SMILES: CN1CCc2[nH]c(-c3cc(Oc4c(F)cc5[nH]ccc5c4S(C)(=O)=O)ccc3F)nc2C1c1cccc(CC(O)CO)c1F] |
| 22 | 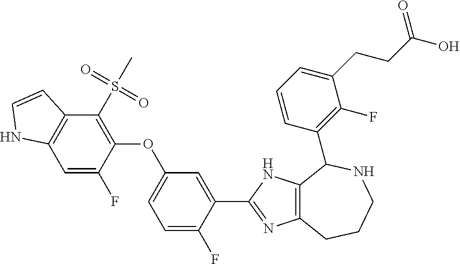 [SMILES: CS(=O)(=O)c1c(Oc2ccc(F)c(-c3nc4c([nH]3)C(c3cccc(CCC(=O)O)c3F)NCCC4)c2)c(F)cc2[nH]ccc12] |

or a pharmaceutically acceptable salt thereof.

30. A pharmaceutical composition comprising a compound of any of the previous claims and a pharmaceutically acceptable excipient.

31. A method of treating a CFTR-mediated disease or disorder comprising administering a patient in need there of a compound any of claims 1-29 or a pharmaceutical composition of claim 30.

32. The method of claim 31, wherein the disease or condition is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear palsy, Pick's disease, several polyglutamine neurological disorders, Huntington's, spinocerebellar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

33. The method of claim 31 or 32, wherein the disease or condition is selected from cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

34. The method of any one of claims 31-33, wherein the disease or condition is cystic fibrosis.

35. A method of treating kidney disease in a subject, comprising administering to the subject a therapeutically effective amount of a compound of any of claims 1-29 or a pharmaceutical composition of claim 30.

36. The method of claim 35, wherein the kidney disease is autosomal dominant polycystic kidney disease or autosomal recessive polycystic kidney disease.

37. The method of claim 35, wherein the kidney disease is autosomal dominant polycystic kidney disease.

38. The method of claim 35, wherein the kidney disease is autosomal recessive polycystic kidney disease.

39. A method of treating cystic fibrosis in a subject, comprising administering to the subject a therapeutically effective amount of a compound of any of claims 1-29 or a pharmaceutical composition of claim 30.

40. The method of claim 39, wherein the subject is human.
